SEQUENCE LISTING (108 sequences)

SEQ ID NO: 1 - protein, 719 aa, Geobacillus stearothermophilus
MKKKTLSLFVGLMLLIGLLFSGSLPYNPNAAEASSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 2 - protein, 499 aa, Aspergillus oryzae
MMVAWWSLFLYGLQVAAPALAATPADWRSQSIYFLLTDRFARTDGSTTATCNTADRKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCLIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

SEQ ID NO: 3 - protein, 515 aa, Saccharomycopsis fibuligera
MIRLTVFLTAVFAAVASCVPVELDKRNTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 4 - protein, 551 aa, Pseudomonas saccharophila
MSHILRAAVLAAVLLPFPALADQAGKSPAGVRYHGGDEIILQGFHWNVVREAPNDWYNILRQQASTIAADGFSAIWMPVPWRDFSSWTDGGKSGGGEGYFWHDFNKNGRYGSDAQLRQAAGALGGAGVKVLYDVVPNHMNRGYPDKEINLPAGQGFWRNDCADPGNYPNDCDDGDRFIGGESDLNTGHPQIYGMFRDELANLRSGYGAGGFRFDFVRGYAPERVDSWMSDSADSSFCVGELWKGPSEYPSWDWRNTASWQQIIKDWSDRAKCPVFDFALKERMQNGSVADWKHGLNGNPDPRWREVAVTFVDNHDTGYSPGQNGGQHHWALQDGLIRQAYAYILTSPGTPVVYWSHMYDWGYGDFIRQLIQVRRTAGVRADSAISFHSGYSGLVATVSGSQQTLVVALNSDLANPGQVASGSFSEAVNASNGQVRVWRSGSGDGGGNDGGEGGLVNVNFRCDNGVTQMGDSVYAVGNVSQLGNWSPASAVRLTDTSSYPTWKGSIALPDGQNVEWKCLIRNEADATLVRQWQSGGNNQVQAAAGASTSGSF

SEQ ID NO: 5 - protein, 926 aa, Bacillus naganoensis
DGNTTNIVVHYFRPSGDYTDWNLWMWPENGDGAEYDFNQPTDSYGEVASVDIPGNPSQVGIIVRKGNWDAKDIDSDRYIDLSKGHEIWLVQGNSQIFYSEKDAEAAAQPAVSNAYLDASNQVLVKLSQPFTLGEGSSGFTVHDDTANKDIPVTSVSDANQVTAVLAGTFQHIFGGSDWAPDNHNTLLKKVNSNLYQFSGNLPEGNYQYKVALNDSWNNPSYPSDNINLTVPAGGAHVTFSYIPSTHAVYDTINNPNADLQVDSSGVKTDLVAVTLGENPDVSHTLSIQTEDYQAGQVIPRKVLDSSQYYYSGDDLGNTYTKNATTFKVWAPTSTQVNVLLYNSATGAVTKTVPMTASGHGVWEATVNQDLENWYYMYEVTGQGSTRTAVDPYATAIAPNGTRGMIVDLAKTDPAGWESDKHITPKNIEDEVIYEMDVRDFSIDSNSGMKNKGKYLALTEKGTKGPDNVKTGVDSLKQLGITHVQLQPVFAFNSVNENDPTQYNWGYDPRNYNVPEGQYATNANGTTRIKEFKEMVLSLHQDHIGVNMDVVYNHTFATQISDFDKIVPEYYYRTDDAGNYTNGSGTGNEIAAERPMVQKFIIDSLKFWVNEYHVDGFRFDLMALLGKDTMSKAATQLHAIDPGIALYGEPWTGGTSALPADQLLTKGAQKGMGVAVFNDNLRNGLDGSVFDSSAQGFATGATGLTDAIKNGVEGSINDFTASPGETINYVTSHDNYTLWDKIAQSNPNDSEADRIKMDELAQAIVMTSQGIPFMQGGEEMLRTKGGNDNSYNAGDVVNEFDWSRKAQYPDVFNYYSGLIHLRLDHPAFRMTTANEINSHLQFLNSPENTVAYELSDHANKDTWGNIVVIYNPNKTAETINLPSGKWEINATSGKVGESTLGQAEGSVQVPGISMMILHQEVSPSDGK

SEQ ID NO: 6 - protein, 829 aa, Bacillus acidopullulyticus
DSTSTEVIVHYHRFDSNYANWDLWMWPYQPVNGNGAAYEFSGKDDFGVKADVQVPGDDTQVGLIVRTNDWSQKNTSDDLHIDLTKGHEIWIVQGDPNIYYNLSDAQAAATPKVSNAYLDNEKTVLAKLTNPMTLSDGSSGFTVTDKTTGEQIPVTAATNANSASSSEQTDLVQLTLASAPDVSHTIQVGAAGYEAVNLIPRNVLNLPRYYYSGNDLGNVYSNKATAFRVWAPTASDVQLLLYNSETGPVTKQLEMQKSDNGTWKLKVPGNLKNWYYLYQVTVNGKTQTAVDPYVRAISVNATRGMIVDLEDTNPPGWKEDHQQTPANPVDEVIYEVHVRDFSIDANSGMKNKGKYLAFTEHGTKGPDNVKTGIDSLKELGINAVQLQPIEEFNSIDETQPNMYNWGYDPRNYNVPEGAYATTPEGTARITQLKQLIQSIHKDRIAINMDVVYNHTFNVGVSDFDKIVPQYYYRTDSAGNYTNGSGVGNEIATERPMVQKFVLDSVKYWVKEYHIDGFRFDLMALLGKDTMAKISKELHAINPGIVLYGEPWTGGTSGLSSDQLVTKGQQKGLGIGVFNDNIRNGLDGNVFDKSAQGFATGDPNQVNVIKNRVMGSISDFTSAPSETINYVTSHDNMTLWDKISASNPNDTQADRIKMDELAQAVVFTSQGVPFMQGGEEMLRTKGGNDNSYNAGDSVNQFDWSRKAQFENVFDYYSWLIHLRDNHPAFRMTTADQIKQNLTFLDSPTNTVAFELKNHANHDKWKNIIVMYNPNKTAQTLTLPSGNWTIVGLGNQVGEKSLGHVNGTVEVPALSTIILHQGTSEDVIDQN

SEQ ID NO: 7 - protein, 776 aa, Pseudomonas amyloderamosa
MKCPKILAALLGCAVLAGVPAMPAHAAINSMSLGASYDAQQANITFRVYSSQATRIVLYLYSAGYGVQESATYTLSPAGSGVWAVTVPVSSIKAAGITGAVYYGYRAWGPNWPYASNWGKGSQAGFVSDVDANGDRFNPNKLLLDPYAQEVSQDPLNPSNQNGNVFASGASYRTTDSGIYAPKGVVLVPSTQSTGTKPTRAQKDDVIYEVHVRGFTEQDTSIPAQYRGTYYGAGLKASYLASLGVTAVEFLPVQETQNDANDVVPNSDANQNYWGYMTENYFSPDRRYAYNKAAGGPTAEFQAMVQAFHNAGIKVYMDVVYNHTAEGGTWTSSDPTTATIYSWRGLDNATYYELTSGNQYFYDNTGIGANFNTYNTVAQNLIVDSLAYWANTMGVDGFRFDLASVLGNSCLNGAYTASAPNCPNGGYNFDAADSNVAINRILREFTVRPAAGGSGLDLFAEPWAIGGNSYQLGGFPQGWSEWNGLFRDSLRQAQNELGSMTIYVTQDANDFSGSSNLFQSSGRSPWNSINFIDVHDGMTLKDVYSCNGANNSQAWPYGPSDGGTSTNYSWDQGMSAGTGAAVDQRRAARTGMAFEMLSAGTPLMQGGDEYLRTLQCNNNAYNLDSSANWLTYSWTTDQSNFYTFAQRLIAFRKAHPALRPSSWYSGSQLTWYQPSGAVADSNYWNNTSNYAIAYAINGPSLGDSNSIYVAYNGWSSSVTFTLPAPPSGTQWYRVTDTCDWNDGASTFVAPGSETLIGGAGTTYGQCGQSLLLLISK

SEQ ID NO: 8 - protein, 500 aa, Thermus thermophilus
MELPRAFGLLLHPTSLPGPYGVGVLGQEARDFLRFLKEAGGRYWQVLPLGPTGYGDSPYQSFSAFAGNPYLIDLRPLAERGYVRLEDPGFPQGRVDYGLLYAWKWPALKEAFRGFKEKASPEEREAFAAFREREAWWLEDYALFMALKGAHGGLPWNRWPLPLRKREEKALREAKSALAEEVAFHAFTQWLFFRQWGALKAEAEALGIRIIGDMPIFVAEDSAEVWAHPEWFHLDEEGRPTVVAGVPPDYFSETGQRWGNPLYRWDVLEREGFSFWIRRLEKALELFHLVRIDHFRGFEAYWEIPASCPTAVEGRWVKAPGEKLFQKIQEVFGEVPVLAEDLGVITPEVEALRDRFGLPGMKVLQFAFDDGMENPFLPHNYPAHGRVVVYTGTHDNDTTLGWYRTATPHEKAFMARYLADWGITFREEEEVPWALMHLGMKSVARLAVYPVQDVLALGSEARMNYPGRPSGNWAWRLLPGELSPEHGARLRAMAEATERL

SEQ ID NO: 9 - DNA, 1641 bp, Artificial Sequence
  Flo1 tether
  CDS (1)..(1641)
aaggacaatagctcgacgattgaaggtagatacccatacgacgttccagactacgctctgcaggctagtggtggtggtggttctggtggtggtggttctggtggtggtggttctgctagcatcagaactccaaccagtgaaggtttggttacaaccaccactgaaccatggactggtacttttacttcgacttccactgaaatgtctactgtcactggaaccaatggcttgccaactgatgaaactgtcattgttgtcaaaactccaactactgccatctcatccagtttgtcatcatcatcttcaggacaaatcaccagctctatcacgtcttcgcgtccaattattaccccattctatcctagcaatggaacttctgtgatttcttcctcagtaatttcttcctcagtcacttcttctctattcacttcttctccagtcatttcttcctcagtcatttcttcttctacaacaacctccacttctatattttctgaatcatctaaatcatccgtcattccaaccagtagttccacctctggttcttctgagagcgaaacgagttcagctggttctgtctcttcttcctcttttatctcttctgaatcatcaaaatctcctacatattcttcttcatcattaccacttgttaccagtgcgacaacaagccaggaaactgcttcttcattaccacctgctaccactacaaaaacgagcgaacaaaccactttggttaccgtgacatcctgcgagtctcatgtgtgcactgaatccatctcccctgcgattgtttccacagctactgttactgttagcggcgtcacaacagagtataccacatggtgccctatttctactacagagacaacaaagcaaaccaaagggacaacagagcaaaccacagaaacaacaaaacaaaccacggtagttacaatttcttcttgtgaatctgacgtatgctctaagactgcttctccagccattgtatctacaagcactgctactattaacggcgttactacagaatacacaacatggtgtcctatttccaccacagaatcgaggcaacaaacaacgctagttactgttacttcctgcgaatctggtgtgtgttccgaaactgcttcacctgccattgtttcgacggccacggctactgtgaatgatgttgttacggtctatcctacatggaggccacagactgcgaatgaagagtctgtcagctctaaaatgaacagtgctaccggtgagacaacaaccaatactttagctgctgaaacgactaccaatactgtagctgctgagacgattaccaatactggagctgctgagacgaaaacagtagtcacctcttcgctttcaagatctaatcacgctgaaacacagacggcttccgcgaccgatgtgattggtcacagcagtagtgttgtttctgtatccgaaactggcaacaccaagagtctaacaagttccgggttgagtactatgtcgcaacagcctcgtagcacaccagcaagcagcatggtaggatatagtacagcttctttagaaatttcaacgtatgctggcagtgccaacagcttactggccggtagtggtttaagtgtcttcattgcgtccttattgctggcaattatttaa

SEQ ID NO: 10 - protein, 546 aa, Artificial Sequence
  Synthetic Construct
KDNSSTIEGRYPYDVPDYALQASGGGGSGGGGSGGGGSASIRTPTSEGLVTTTTEPWTGTFTSTSTEMSTVTGTNGLPTDETVIVVKTPTTAISSSLSSSSSGQITSSITSSRPIITPFYPSNGTSVISSSVISSSVTSSLFTSSPVISSSVISSSTTTSTSIFSESSKSSVIPTSSSTSGSSESETSSAGSVSSSSFISSESSKSPTYSSSSLPLVTSATTSQETASSLPPATTTKTSEQTTLVTVTSCESHVCTESISPAIVSTATVTVSGVTTEYTTWCPISTTETTKQTKGTTEQTTETTKQTTVVTISSCESDVCSKTASPAIVSTSTATINGVTTEYTTWCPISTTESRQQTTLVTVTSCESGVCSETASPAIVSTATATVNDVVTVYPTWRPQTANEESVSSKMNSATGETTTNTLAAETTTNTVAAETITNTGAAETKTVVTSSLSRSNHAETQTASATDVIGHSSSVVSVSETGNTKSLTSSGLSTMSQQPRSTPASSMVGYSTASLEISTYAGSANSLLAGSGLSVFIASLLLAII

SEQ ID NO: 11 - DNA, 807 bp, Artificial Sequence
  Sed1 tether
  CDS (1)..(807)
aaggacaatagctcgacgattgaaggtagatacccatacgacgttccagactacgctctgcaggctagtggtggtggtggttctggtggtggtggttctggtggtggtggttctgctagcgctcttccaactaacggtacttctactgaagctccaactgatactactactgaagctccaaccaccggtcttccaaccaacggtaccacttcagctttcccaccaactacatctttgccaccaagcaacactaccaccactcctccttacaacccatctactgactacaccactgactacactgtagtcactgaatatactacttactgtccagaaccaaccactttcaccacaaacggtaagacttacaccgtcactgaaccaaccacattgactatcactgactgtccatgcaccattgaaaagccaacaaccacatcaaccaccgaatacactgtagtcactgagtacactacttactgtccagaaccaaccactttcaccacaaacggtaagacttacaccgtcactgaaccaaccactttgactatcactgactgtccatgtactattgaaaagagcgaagcccctgagtcttctgtcccagttaccgaatctaagggcactaccaccaaagaaacaggtgttactaccaaacaaaccacagccaacccaagtctaaccgtctccacagtcgtcccagtttcatcctctgcttcttctcattccgttgtcatcaacagtaacggtgctaacgtcgtcgttccaggtgctttaggtttggctggtgttgctatgttattctaa

SEQ ID NO: 12 - protein, 268 aa, Artificial Sequence
  Synthetic Construct
KDNSSTIEGRYPYDVPDYALQASGGGGSGGGGSGGGGSASALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLF

SEQ ID NO: 13 - DNA, 759 bp, Artificial Sequence
  Tir1 tether
  CDS (1)..(759)
aaggacaatagctcgacgattgaaggtagatacccatacgacgttccagactacgctctgcaggctagtggtggtggtggttctggtggtggtggttctggtggtggtggttctgctagcagcttagcttctgattcttcctctggattttccttaagcagtatgccagctggtgttttggatatcggtatggctttagcttccgccactgacgactcctacactactttgtactctgaggttgactttgctggtgttagcaagatgttgaccatggttccatggtactcctctagattggaaccagctttgaagtctttgaatggtgatgcttcttcttctgctgccccaagctcttctgctgctccaacttcttctgctgccccaagctcatctgctgccccaacttcttctgctgcctcaagctcttctgaagctaagtcttcttctgctgccccaagctcttctgaagctaagtcttcttctgctgccccaagctcttctgaagctaagtcttcttctgctgccccaagctcttctgaagctaagtcttcttctgctgctccaagctccactgaagctaagataacttctgctgctccaagctccactggtgccaagacctctgccatctctcaaattaccgatggtcaaatccaagctaccaaggctgtttctgagcaaactgaaaacggtgctgctaaggcctttgttggtatgggtgctggtgttgtcgcagctgccgctatgttgttataa

SEQ ID NO: 14 - protein, 252 aa, Artificial Sequence
  Synthetic Construct
KDNSSTIEGRYPYDVPDYALQASGGGGSGGGGSGGGGSASSLASDSSSGFSLSSMPAGVLDIGMALASATDDSYTTLYSEVDFAGVSKMLTMVPWYSSRLEPALKSLNGDASSSAAPSSSAAPTSSAAPSSSAAPTSSAASSSSEAKSSSAAPSSSEAKSSSAAPSSSEAKSSSAAPSSSEAKSSSAAPSSTEAKITSAAPSSTGAKTSAISQITDGQIQATKAVSEQTENGAAKAFVGMGAGVVAAAAMLL

SEQ ID NO: 15 - DNA, 450 bp, Artificial Sequence
  Cwp2 tether
  CDS (1)..(450)
aaggacaatagctcgacgattgaaggtagatacccatacgacgttccagactacgctctgcaggctagtggtggtggtggttctggtggtggtggttctggtggtggtggttctgctagcggatccggtggcggtggatctggaggaggcggttcttggtctcacccacaatttgaaaagggtggagaaaacttgtactttcaaggcggtggtggaggttctggcggaggtggctccggctcagctatctctcaaatcaccgacggtcaaatccaagccactaccacagctaccactgaagctacaactaccgctgctccttcatctactgttgaaactgtttctccatcttccaccgaaaccatctctcaacaaaccgaaaacggtgctgctaaggctgctgttggtatgggtgctggtgctttggctgctgctgctatgttgttgtaa

SEQ ID NO: 16 - protein, 149 aa, Artificial Sequence
  Synthetic Construct
KDNSSTIEGRYPYDVPDYALQASGGGGSGGGGSGGGGSASGSGGGGSGGGGSWSHPQFEKGGENLYFQGGGGGSGGGGSGSAISQITDGQIQATTTATTEATTTAAPSSTVETVSPSSTETISQQTENGAAKAAVGMGAGALAAAAMLL

SEQ ID NO: 17 - DNA, 468 bp, Artificial Sequence
  Ccw12 tether
  CDS (1)..(468)
aaggacaatagctcgacgattgaaggtagatacccatacgacgttccagactacgctctgcaggctagtggtggtggtggttctggtggtggtggttctggtggtggtggttctgctagcgctgctaacgttaccactgctactgtcagccaagaatctaccactttggtcaccatcacttcttgtgaagaccacgtctgttctgaaactgtctccccagctttggtttccaccgctaccgtcaccgtcgatgacgttatcactcaatacaccacctggtgcccattgaccactgaagccccaaagaacggtacttctactgctgctccagttacctctactgaagctccaaagaacaccacctctgctgctccaactcactctgtcacctcttacactggtgctgctgctaaggctttgccagctgctggtgctttgttggctggtgccgctgctttgttgttgtaa

SEQ ID NO: 18 - protein, 155 aa, Artificial Sequence
  Synthetic Construct
KDNSSTIEGRYPYDVPDYALQASGGGGSGGGGSGGGGSASAANVTTATVSQESTTLVTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 19 - DNA, 510 bp, Artificial Sequence
  Spi1 tether
  CDS (1)..(510)
aaggacaatagctcgacgattgaaggtagatacccatacgacgttccagactacgctctgcaggctagtggtggtggtggttctggtggtggtggttctggtggtggtggttctgctagcttggtatctaattctagttcctctgtaatcgtggtaccatcaagcgatgctactattgccggtaacgatacagccacgccagcaccagagccatcatccgccgctccaatattctacaactcgactgctactgcaacacagtacgaagttgtcagtgaattcactacttactgcccagaaccaacgactttcgtaacgaatggcgctacattcactgttactgccccaactacgttaacaattaccaactgtccttgcactatcgagaagcctacttcagaaacatcggtttcttctacacatgatgtggagacaaattctaatgctgctaacgcaagagcaatcccaggagccctaggtttggctggtgcagttatgatgcttttatga

SEQ ID NO: 20 - protein, 169 aa, Artificial Sequence
  Synthetic Construct
KDNSSTIEGRYPYDVPDYALQASGGGGSGGGGSGGGGSASLVSNSSSSVIVVPSSDATIAGNDTATPAPEPSSAAPIFYNSTATATQYEVVSEFTTYCPEPTTFVTNGATFTVTAPTTLTITNCPCTIEKPTSETSVSSTHDVETNSNAANARAIPGALGLAGAVMMLL

SEQ ID NO: 21 - DNA, 1398 bp, Artificial Sequence
  Pst1 tether
  CDS (1)..(1398)
aaggacaatagctcgacgattgaaggtagatacccatacgacgttccagactacgctctgcaggctagtggtggtggtggttctggtggtggtggttctggtggtggtggttctgctagcgctacttcctcttcttccagcataccctcttcctgtaccataagctcacatgccacggccacagctcagagtgacttagataaatatagccgctgtgatacgttagtcgggaacttaactattggtggtggtttgaagactggtgctttggctaatgttaaagaaatcaacgggtctctaactatatttaacgctacaaatctaacctcattcgctgctgattccttggagtccatcacagattctttgaacctacagagtttgacaatcttgacttctgcttcatttgggtctttacagagcgttgatagtataaaactgattactctacccgccatctccagttttacttcaaatatcaaatctgctaacaacatttatatttccgacacttcgttacaatctgtcgatggattctcagccttgaaaaaagttaacgtgttcaacgtcaataacaataagaaattaacctcgatcaaatctccagttgaaacagtcagcgattctttacaattttcgttcaacggtaaccagactaaaatcaccttcgatgacttggtttgggcaaacaatatcagtttgaccgatgtccactctgtttccttcgctaacttgcaaaagattaactcttcattgggtttcatcaacaactccatctcaagtttgaatttcactaagctaaacaccattggccaaaccttcagtatcgtttccaatgactacttgaagaacttgtcgttctctaatttgtcaaccataggtggtgctcttgtcgttgctaacaacactggtttacaaaaaattggtggtctcgacaacctaacaaccattggcggtactttggaagttgttggtaacttcacctccttgaacctagactctttgaagtctgtcaagggtggcgcagatgtcgaatcaaagtcaagcaatttctcctgtaatgctttgaaagctttgcaaaagaaagggggtatcaagggtgaatcttttgtctgcaaaaatggtgcatcatccacatctgttaaactatcgtccacttccaaatctcaatcaagccaaactactgccaaggtttccaagtcatcttctaaggccgaggaaaagaagttcacttctggcgatatcaaggctgctgcttctgcctctagtgtttctagttctggcgcttccagctctagctctaagagttccaaaggcaatgccgctatcatggcaccaattggccaaacaacccctttggtcggtcttttgacggcaatcatcatgtctataatgtaa

SEQ ID NO: 22 - protein, 465 aa, Artificial Sequence
  Synthetic Construct
KDNSSTIEGRYPYDVPDYALQASGGGGSGGGGSGGGGSASATSSSSSIPSSCTISSHATATAQSDLDKYSRCDTLVGNLTIGGGLKTGALANVKEINGSLTIFNATNLTSFAADSLESITDSLNLQSLTILTSASFGSLQSVDSIKLITLPAISSFTSNIKSANNIYISDTSLQSVDGFSALKKVNVFNVNNNKKLTSIKSPVETVSDSLQFSFNGNQTKITFDDLVWANNISLTDVHSVSFANLQKINSSLGFINNSISSLNFTKLNTIGQTFSIVSNDYLKNLSFSNLSTIGGALVVANNTGLQKIGGLDNLTTIGGTLEVVGNFTSLNLDSLKSVKGGADVESKSSNFSCNALKALQKKGGIKGESFVCKNGASSTSVKLSSTSKSQSSQTTAKVSKSSSKAEEKKFTSGDIKAAASASSVSSSGASSSSSKSSKGNAAIMAPIGQTTPLVGLLTAIIMSIM

SEQ ID NO: 23 - DNA, 330 bp, Artificial Sequence
  Aga1/2 tether
  CDS (1)..(330)
aaggacaatagctcgacgattgaaggtagatacccatacgacgttccagactacgctctgcaggctagtggtggtggtggttctggtggtggtggttctggtggtggtggttctgctagccaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttaa

SEQ ID NO: 24 - protein, 109 aa, Artificial Sequence
  Synthetic Construct
KDNSSTIEGRYPYDVPDYALQASGGGGSGGGGSGGGGSASQELTTICEQIPSPTLESTPYSLSTTTILANGKAMQGVFEYYKSVTFVSNCGSHPSTTSKGSPINTQYVF

SEQ ID NO: 25 - DNA, 381 bp, Artificial Sequence
  Aga1/2 tether
  CDS (1)..(381)
atgcagttacttcgctgtttttcaatattttctgttattgcttcagttttagcacaggagctgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcgattctcacccctcaacaactagcaaagacagccccataaacacacagtatgtttttaaggacaatagctcgacgattgaaggtagatacccatacgacgttccagactacgctctgcaggctagtggtggtggtggttctggtggtggtggttctggtggtggtggttctgctagc

SEQ ID NO: 26 - protein, 127 aa, Artificial Sequence
  Synthetic Construct
MQLLRCFSIFSVIASVLAQELTTICEQIPSPTLESTPYSLSTTTILANGKAMQGVFEYYKSVTFVSNCDSHPSTTSKDSPINTQYVFKDNSSTIEGRYPYDVPDYALQASGGGGSGGGGSGGGGSAS

SEQ ID NO: 27 - DNA, 750 bp, Artificial Sequence
  Promoter of tdh1 gene
agaaacgaatgtatatgctcatttacactctatatcaccatatggaggataagttgggctgagcttctgatccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttacgctaggaataggatatgcgacgaagacgcttctgcttagtaaccacaccacattttcaaggggtcgatctgcttgcttcctttactgtcacgagcggcccataatcgcgctttttttttaaaatgcgcgagacagcaaacaggaagctcgggtttcaaccttcggagtggtcgcagatctggagactggatccttacaatacagtaaggcaagccaccatctgcttcttaggtgcatgcgacggtatccacgtgcagaacaacatagtctgaagaagggggggaggagcatgttcattctctgtagcactaagagcttggtgataatgaccaaaactggagtctcgaaatcatataaatagacaatatattttcacacaatgtgatttgtagtacagttctactctctctcttgcataaataagaaattcatcaagaacttggtttgatatttcaccaacacacacaaaaaacagtacttcactaaatttacacacaaaacaaa

SEQ ID NO: 28 - DNA, 750 bp, Artificial Sequence
  Promotoer of hor7 gene
acctccatgaaatttttttttttctttcgattagcacgcacacacatcacatagactgcgtcataaaaatacactacggaaaaaccataaagagcaaagcgatacctacttggaaggaaaaggagcacgcttgtaagggggatgggggctaagaagtcattcactttcttttcccttcgcggtccggacccgggacccctcctctccccgcacaatttcttcctttcatatcttccttttattcctatcccgttgaagcaaccgcactatgactaaatggtgctggacatctccatggctgtgacttgtgtgtatctcacagtggtaacggcaccgtggctcggaaacggttccttcgtgacaattctagaacaggggctacagtctcgataatagaataataagcgcatttttgttagcgccgccgcggcgcccgtttcccaatagggaggcgcagtttatcggcggagctttacttcttcctatttgggtaagcccctttctgttttcggccagtggttgctgcaggctgcgccggagaacatagtgataagggatgtaactttcgatgagagaattagcaagcggaaaaaaaactatggctagctgggagttgtttttcaatcatataaaagggagaaattgttgctcactatgtgacagtttctgggacgtcttaacttttattgcagaggactatcaaatcatacagatattgtcaaaaaaaaaaaaaaagactaataataaaaa

SEQ ID NO: 29 - DNA, 750 bp, Artificial Sequence
  Promoter of hsp150 gene
tgtaaaaacaagcaaaaaaaaaccaagaaggaacaaatgcaccaaactgttgatctattctgcaaaaaaagtatggtaaattttttccattatcctggccgctaatccatatggaggtgaacttagaacttgcacaaggatgcgatgaatgataggctttgtgctataattaatgcaggcaggtccgccatgtccaacacgttgctggccgcaaaacgagtcaatctcactgctttgccacgctcatttctccccccttctgcccaattaggcgaccctcacaatgcacatacacatttcccacctctattggaaggggccgtaaatggtaattcttgggagttattcatattaagtgatcttactatttcctatttcggaaattattaaagacaaaaaagctcatttatggctttccgtctgtagtgataagtcgccaactcagcctaatttttcatttctttaccagatcaggaaaactaatagtacaaatgagttttttctcaagcggaacaccacattttgagctaaatttagattttggtcaaaataagaaagatcctaaaaaaggaatggttggtgaaaaatttattagcttgaatggtaggaatcctcgagatataaaaggaacacttgaagtctaacgacaatcaatttcgattatgtccttccttttacctcaaagctcaaaaaaacatcaataagaaactcatattccttttctaaccctagtacaataataataatata

SEQ ID NO: 30 - DNA, 450 bp, Artificial Sequence
  Promoter of hxt7 gene
ccagaaaggcaacgcaaaattttttttccagggaataaactttttatgacccactacttctcgtaggaacaatttcgggcccctgcgtgttcttctgaggttcatcttttacatttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaaaaaaagtataaatagagacgatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaa

SEQ ID NO: 31 - DNA, 1000 bp, Artificial Sequence
  Promoter of gpm1 gene
tgtggtagaattcaaaagactatgtgatgccataggcaagaagggagactctcactccgagatgggcagcttgatcgcccaggaattgaattgtattgtggtggagaaaggtcagtcagataagatattctcacccgatagtgaaaaagacatgttgacgaacagcgaagagggcagcaacaagagggtaggaggccaaggtgatactttgacaggagctatatcatgcatgcttgcatttagtcgtgcaatgtatgactttaagatttgtgagcaggaagaaaagggagaatcttctaacgataaacccttgaaaaactgggtagactacgctatgttgagttgctacgcaggctgcacaattacacgagaatgctcccgcctaggatttaaggctaagggacgtgcaatgcagacgacagatctaaatgaccgtgtcggtgaagtgttcgccaaacttttcggttaacacatgcagtgatgcacgcgcgatggtgctaagttacatatatatatatatatatatatatatatatatatagccatagtgatgtctaagtaacctttatggtatatttcttaatgtggaaagatactagcgcgcgcacccacacacaagcttcgtcttttcttgaagaaaagaggaagctcgctaaatgggattccactttccgttccctgccagctgatggaaaaaggttagtggaacgatgaagaataaaaagagagatccactgaggtgaaatttcagctgacagcgagtttcatgatcgtgatgaacaatggtaacgagttgtggctgttgccagggagggtggttctcaacttttaatgtatggccaaatcgctacttgggtttgttatataacaaagaagaaataatgaactgattctcttcctccttcttgtcctttcttaattctgttgtaattaccttcctttgtaattttttttgtaattattcttcttaataatccaaacaaacacacatattacaata

SEQ ID NO: 32 - DNA, 750 bp, Artificial Sequence
  Promoter of pgk1 gene
acgcacagatattataacatctgcacaataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 33 - DNA, 1020 bp, Artificial Sequence
  Promoter of stl1 gene
tggtctcgcgtgtgaatcagcaatgattctgaaatactccttttacaacctttgcaaagataatgtcattcagtctgatattacgagcgacctggaagcactaatccatattcttcattcaacttactccattttccttggcaaacaatgccccacaatcatatacgtcataactataagggatatgtctggaatgcggccaagatagaattaaagggctgcagaacaccactactgatactcattgccaaggctaggaggcaccatccgtttcattttctttgaggtaagccaatcatgaaatagtatacacatccataacggacgtacggacgaaataagtgccgttgtcccactattccaccgcatttggcccatttggctcactttgactcaacttgcgtcattttaactgatatgaagggtccgactttgtcctttttcggccaccgcataccccacggcgacgcctccgctacctgcatttgagtagcatctccgtttcgcggggtattcggcgctacgtcgcctgttcgagcggctctgttcgttgcatgaaactaaaataagcggaaagtgtccagccatccactacgtcagaaagaaataatggttgtacactgtttctcggctatataccgtttttggttggttaatcctcgccaggtgcagctattgcgcttggctgcttcgcgatagtagtaatctgagaaagtgcagatcccggtaagggaaacacttttggttcacctttgatagggctttcattggggcattcgtaacaaaaaggaagtagatagagaaattgagaaagcttaagtgagatgttttagcttcaattttgtccccttcaacgctgcttggccttagagggtcagaattgcagttcaggagtagtcacactcatagtatataaacaagccctttattgattttgaataattattttgtatacgtgttctagcatacaagttagaataaataaaaaatagaaaaatagaacatagaaagttttagacc

SEQ ID NO: 34 - DNA, 500 bp, Artificial Sequence
  Terminator of dit1 gene
taaagtaagagcgctacattggtctacctttttgttcttttacttaaacattagttagttcgttttctttttctcatttttttatgtttcccctcaaaagttctgattttataatattttatttcacacaattccaattaacagagggggaatagattctttagcttagaaaattagtgatcaatatatatttgcctttcttttcatcttttcagtgatattaatggtttcgagacactgcaatggccctagttgtctaagaggatagatgttactgtcaaagatgatattttgaatttcaattgacgtaattaatgatactattaataatacagagcgtatatgaagtattgcaaataacatgcacagttcttttgggatgagaatgagaatgagaggcgaaggcgggcgttcagaaaagcgttgcggagtaacaagtgattaaatagcacccaaataatcttctttgatactaccgattgcgtgaatagaactcactt

SEQ ID NO: 35 - DNA, 500 bp, Artificial Sequence
  Terminator of idp1 gene
tcgaatttacgtagcccaatctaccactttttttttcattttttaaagtgttatacttagttatgctctaggataatgaactacttttttttttttttactgttatcataaatatatataccttattgatgtttgcaaccgtcggttaattccttatcaaggttccccaagttcggatcattaccatcaatttccaacatcttcatgagttcttcttcttcattaccgtgttttagggggctgttcgcacttctaatagggctatcaccaagctgttctaattcgtccaaaagttcagtaacacgatctttatgcttcagttcgtcataatctttcaattcataaatatttacaatttcgtctacgatattaaattgcctcttgtaggtgcctatcttttccttatgctcttcattttcaccgttttcttgaaaccaaacaccgaactcactacgcatttctttcataggctcatataatacttcttttgacgtcatttg

SEQ ID NO: 36 - DNA, 401 bp, Artificial Sequence
  Terminator of gpm1 gene
gtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaataaccttgatgtttacatcgtagtttaatgtacaccccgcgaattcgttcaagtaggagtgcaccaattgcaaagggaaaagctgaatgggcagttcgaatag

SEQ ID NO: 37 - DNA, 500 bp, Artificial Sequence
  Terminator of pma1 gene
tcctgttgaagtagcatttaatcataatttttgtcacattttaatcaacttgatttttctggtttaatttttctaattttaattttaatttttttatcaatgggaactgatacactaaaaagaattaggagccaacaagaataagccgcttatttcctactagagtttgcttaaaatttcatctcgaattgtcattctaatattttatccacacacacaccttaaaatttttagattaaatggcatcaactcttagcttcacacacacacacacaccgaagctggttgttttatttgatttgatataattggtttctctggatggtactttttctttcttggttatttcctattttaaaatatgaaacgcacacaagtcataattattctaatagagcacaattcacaacacgcacatttcaactttaatatttttttagaaacactttatttagtctaattcttaatttttaatatatataatgcacacacactaattt

SEQ ID NO: 38 - DNA, 500 bp, Artificial Sequence
  Terminator of tdh3 gene
gtgaatttactttaaatcttgcatttaaataaattttctttttatagctttatgacttagtttcaatttatatactattttaatgacattttcgattcattgattgaaagctttgtgttttttcttgatgcggtattgcattgttcttgtctttttcgccacatgtaatatctgtaatagatatctgatacattgtggatgctgagtgaaattttagttaataatggaggcgctcttaataattttggggatattggcttttttttttaaagtttacaaatgaattttttccgccaggataacgattctgaagttactcttagcgttcctatcggtacagccatcaaatcatgcctataaatcatgcctatatttgcgtgcagtcagtatcatctacatgaaaaaaactcccgcaatttcttatagaatacgttgaaaattaaatgtacgcgccaagataagataacataaatctagatgcagtaatatacacagattcc

SEQ ID NO: 39 - DNA, 500 bp, Artificial Sequence
  Terminator of hxt2 gene
gagattatacttaaactagcactgatttttttaaggctaatggctactaatactttaatagatgatcttcatacttttttatttaacgatttttaatgatgtttttatttgtaccactcatttatctagatttttttaatactgatcaaatcttacggactcgacgttaaaaagttcctacatacgtctggtacttgaaacgctgcttcgaggtattgacactataagaatacgatccaaatacttacaccgcatgtaaaaatatgccgacaatatgaatacttgttgatgaatgatatttgattttaatccggcaatttacctcctttatataatccaataattgttgataattagtggttaggttgcagtactaataagaattaagacaaatattcttctactatataaaaggtgcaaacaaaacacacgccgatcggccatactaaacaagaccaacataataatggtggaaccatttactgtattttcaatgtaac

SEQ ID NO: 40 - DNA, 500 bp, Artificial Sequence
  Terminator of ira2 gene
aggtgttacataaactaatgaaagaaatatcaatatctatctgtaagcatgaatgtacatatctcatgttagggttttcttatcgctaatttttcgcaatttgttacgtgggttgcttttatacagctacaatttttatatattctatcatgtaatgaatggctcagtaaattcaagcgccacatagactaatgtacataccaatgcatttaattgtaagaatgaaaggggccattcatctaccgtcttagttgaaagtgtttctgtgaattttttcaaattccgttttttcctttttatataatagcatggtggcgcgagcatcttcgactgaagaatgctcaccttcttgaatggaaatttttaaaacctccttggttaatttctttaagctgggtgttttacccttagcatacaacttcctgaatgggaggtgtcttgaagtgtccctgagtagtgactttgggtgggataacatcaatgcttcgagatcatgcttt

SEQ ID NO: 41 - protein, 5 aa, Artificial Sequence
  G4S motif
GGGGS

SEQ ID NO: 42 - protein, 457 aa, Penicillium funiculosum
MLRYLSIVAATAILTGVEAQQSVWGQCGGQGWSGATSCAAGSTCSTLNPYYAQCIPGTATSTTLVKTTSSTSVGTTSPPTTTTTKASTTATTTAAASGNPFSGYQLYANPYYSSEVHTLAIPSLTGSLAAAATKAAEIPSFVWLDTAAKVPTMGTYLANIEAANKAGASPPIAGIFVVYDLPDRDCAAAASNGEYTVANNGVANYKAYIDSIVAQLKAYPDVHTILIIEPDSLANMVTNLSTAKCAEAQSAYYECVNYALIKPHLAHVAMYIDAGHAGWLGWSANLSPAAQLFATVYKNASAPASLRGLATNVANYNAWSISSPPSYTSGDSNYDEKLYINALSPLLTSNGWPDAHFIMDTSRNGVQPTKQQAWGDWCNVIGTGFGVQPTTNTGDPLEDAFVWVKPGGESDGTSNSSATRYDFHCGYSGALQPAPEAGTWFQAYFVQLLTNANPALV

SEQ ID NO: 43 - protein, 408 aa, Rasamsonia emersonii
MVRLSPVLLASIAGSGLPLAQAAGLNTAAKAIGLKYFGTATDNPELSDTAYETQLNNTQDFGQLTPANSMKWDATEPEQNVFTFSAGDQIANLAKANGQMLRCHNLVWYNQLPSWVTSGSWTNETLLAAMKNHITNVVTHYKGQCYAWDVVNEALNDDGTYRSNVFYQYIGEAYIPIAFATAAAADPNAKLYYNDYNIEYPGAKATAAQNLVKLVQSYGARIDGVGLQSHFIVGETPSTSSQQQNMAAFTALGVEVAITELDIRMQLPETEALLTQQATDYQSTVQACANTKGCVGITVWDWTDKYSWVPSTFSGYGDACPWDANYQKKPAYEGILTGLGQTVTSTTYIISPTTSVGTGTTTSSGGSGGTTGVAQHWEQCGGLGWTGPTVCASGYTCTVINEYYSQCL

SEQ ID NO: 44 - protein, 605 aa, Aspergillus niger
MQTLLVSSLVVSLAAALPHYIRSNGIEASLLTDPKDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGVNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAKHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQ

SEQ ID NO: 45 - protein, 291 aa, Thermomyces lanuginosus
MRSSLVLFFLSAWTALARPVRRAVPQDLLDQFELFSQYSAAAYCAANNHAPVGSDVTCSENVCPEVDAADATFLYSFEDSGLGDVTGLLALDNTNKLIVLSFRGSRSVENWIANLAADLTEISDICSGCEGHVGFVTSWRSVADTIREQVQNAVNEHPDYRVVFTGHSLGGALATIAAAALRGNGYNIDVFSYGAPRVGNRAFAEFLTAQTGGTLYRITHTNDIVPRLPPRDWGYSHSSPEYWVTSGNDVPVTANDITVVEGIDSTDGNNQGNIPDIPSHLWYFGPISECD

SEQ ID NO: 46 - protein, 146 aa, Sus scrofa
MKFLVLAVLLTVGAAQEGISSRALWQFRSMIKCAIPGSHPLMDFNNYGCYCGLGGSGTPVDELDRCCETHDNCYRDAKNLDSCKFLVDNPYTESYSYSCSNTEITCNSKNNACEAFICNCDRNAAICFSKAPYNKEHKNLDTKKYC

SEQ ID NO: 47 - protein, 150 aa, Streptomyces vialaceoruber
MRTTTRTRTTLAAVGAALALGVAAAPPQAAPADKPQVLASFTQTSASSQNAWLAANRNQSAWAAYEFDWSTDLCSQAPDNPFGFPFNTACARHDFGYRNYKAAGSFDANKSRIDSAFYEDMKRVCTGYTGEKNTACNSTAWTYYQAVKIL

SEQ ID NO: 48 - protein, 295 aa, Aspergillus niger
MFSLARLGTVAGLFLLAQAAPASLRRDVSSSLLNNLDLFAQYSAAAYCDENLNSTGTKLTCSVGNCPLVEAASTQSLDEFNESSSYGNPAGYLAADETNKLLVLSFRGSADLANWVANLNFGLEDASDLCSGCEVHSGFWKAWSEIADTITSKVESALSDHSDYSLVLTGHSYGAALAALAATALRNSGHSVELYNYGQPRLGNEALATYITDQNKGGNYRVTHTNDIVPKLPPTLLGYHHFSPEYYISSADEATVTTTDVTEVTGIDATGGNDGTDGTSIDAHRWYFIYISECS

SEQ ID NO: 49 - protein, 515 aa, Artificial Sequence
  Mutated glucoamylase
MIRLTVFLTAVFAAVASCVPVELDKRNTGHFQAYSGYTVNRSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 50 - protein, 652 aa, Artificial Sequence
  Mutated alpha-amylase
MLLQAFLFLLAGFAAKISAGPAAANAETANKSNNVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQVYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQVFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYTFVKKDPSQTNIIGYQNPDHWGQVNAYIYKHDGGRAIELTGSWPGKAMTKNANGMYTLTLPENTDTANAKVIFNNGSAQVPGQNQPGFDYVQNGLYNNSGLNGYLPH

SEQ ID NO: 51 - protein, 713 aa, Artificial Sequence
  Mutated maltogenic alpha-amylase
MLLQAFLFLLAGFAAKISAYNPNAAEASSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 52 - protein, 19 aa, Saccharomyces cerevisiae
MLLQAFLFLLAGFAAKISA

SEQ ID NO: 53 - protein, 22 aa, Artificial Sequence
  HA tag
KDNSSTIEGRYPYDVPDYALQA

SEQ ID NO: 54 - protein, 18 aa, Artificial Sequence
  Modified G4S linker
SGGGGSGGGGSGGGGSAS

SEQ ID NO: 55 - DNA, 750 bp, Artificial Sequence
  Promoter of trx2 gene
ataaccacaatgatcgcagaccgtcgacatgataatgacttctttaaagtgtgggatatttactggcttcatatgagtttcacactcttgggtacacgatggacaagaagctctgaatgtttgcacctcgttgttaaagttttcgatgtcggtggcgtctgacaaaaactgtgggtttgctgagccaactttgacagattcagaaggatttctttcacggttggccaattgttttaattgttcttggcgacgttgctccaactggtctctagtaataatgccaacttgaacgttttgttcgtcggttctcacgtactgggtatgagaccatttgtgttgaggttcaccgggtttgtattcgatccaggaatttcccgcaggatcatccaaaataaatgtaatcggaatagtgttgggttcacaattgatgtaggatttaactttctgtacgaagtcatcgatcttcttgtaaagagcttcgtctatagattttctcatttcctggtcttgcgacagatcgtcgatcatctcggataacaaaccttcaactgtcgtcaattgacctctcttagcaggaatctcaatgtctagctcgacaaacttacaagtggcagtttctgacttaataacttgcctgttaaaatcttcacggcactccactttcaaaacgtaacgagagcccttctcttgaatttgagaagcgggttggatctcacagttcttaaacccacagtgaggacagtcgaatga

SEQ ID NO: 56 - DNA, 750 bp, Artificial Sequence
  Promoter of sti1 gene
taccacaaagggtaaaacgtcaataaaagcaagcaatgttattcgtgccagagaactatatctggcgtacgtcttatatataacacaagcaagggcaaatacgtggttagtggtgtaaaaactgaagggattaatttacatacaactagatccatctttctcaaaaatgacttcagtatcgccctcgccacctgccagtcgatcgggctcaatgtgctccgacttaccgtcctctttgcagactgagaaactggcacatattataggtcttgatgccgacgatgaagttctccggcgcgtaaccaagcagttgagcagatctaggagaattgcttgtctgactggggcaggcatttcgtgcaacgcgggcattcctgactttcgctcttctgatgggctctacgacctagtgaaaaaggattgttcacagtattggtctatcaagtccggcagggaaatgtttgatatttcgctatttagagatgacttcaaaatatccatttttgctaaatttatggagaggctctattcaaatgttcaattggcaaagccgactaagacgcacaagttcattgcgcatctaaaagataggaacaaactgctgcgctgttacacgcaaaacatcgatgggctcgaagaaagcataggacttactttatcaaataggaaattaccgcttacctcatttagttcacattggaaaaatctggatgtcgttcagttgcacggcgacctgaaaactctttcgtg

SEQ ID NO: 57 - DNA, 750 bp, Artificial Sequence
  Promoter of gdp1 gene
aaggaaaatatatactctttcccaggcaaggtgacagcggtccccgtctcctccacaaaggcctctcctggggtttgagcaagtctaagtttacgtagcataaaaattctcggattgcgtcaaataataaaaaaagtaactccacttctacttctacatcggaaaaacattccattcacatatcgtctttggcctatcttgttttgtccttggtagatcaggtcagtacaaacgcaacacgaaagaacaaaaaaagaagaaaaacagaaggccaagacagggtcaatgagactgttgtcctcctactgtccctatgtctctggccgatcacgcgccattgtccctcagaaacaaatcaaacacccacaccccgggcacccaaagtccccacccacaccaccaatacgtaaacggggcgccccctgcaggccctcctgcgcgcggcctcccgccttgcttctctcccctcccttttctttttccagttttccctattttgtccctttttccgcacaacaagtatcagaatgggttcatcaaatctatccaacctaattcgcacgtagactggcttggtattggcagtttcgcagttatatatatactaccatgagtgaaactgttacgttaccttaaattctttctccctttaattttcttttatcttactctcctacataagacatcaagaaacaattgtatattgtacacccccccccctccacaaacacaaatattgataatataaag

SEQ ID NO: 58 - DNA, 750 bp, Artificial Sequence
  Promoter of cup1 gene
taaatatatgctgagatttagcaagaaaaaacaaaattttgcgagaaactctgataacaataatgttacagattatagtcagtcggcgaagaacaaaaatgttctcttgaaattccccgttagtgaactgaacagaatctatttaaaatttaaggagatttcagattttttaatggaaagagaagttgtccaaaggagtataattattgacaaggatttggaatctgataatctgggtattactacggcaaacttcaacgatttctatgatgcattttataattagtaagccgatcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcacataaa

SEQ ID NO: 59 - DNA, 750 bp, Artificial Sequence
  Promoter of glo1 gene
tattaatccaaaaagtgaatctttgacacccctttctcctatcttgcttagtaccgagtgatcttttgcataatcaaaacaagtgaagaaggtcgagttttgaaccttctctaatataagtcgacggtctttcgtgatggcgtcaatattacttagtcttttgtacttcacatataccattaatggcgtcaatcccttataattttcctcattaatatctagcttagtcagttgtaaaaggattggaatgtttgttttcaaaacatgcaacaagctattgcccttattatcggtgtggtctagatagtcaaacaaactgttgtgctttctataccaagtatttgcgatatcaaatgcggtcttcaccatttcttcataatttggttgatcataacttctaaagatggaaaataaaggcgtttgtccactggagttttttcgtttccaatcaatataatttccgatgcttttaagtatatccatttcatgggttagataatgagccacggtacgtgcatatttatctgttttattgatataagaaaccagctcctcttcagtacaattgaacagcatgatctttatgagaactttcgcggcttctaaatttcctgcttttatcgattctataagcagcgtagaaccatcaatcgtttcgtcctctaaaaggtcttctactggaaagtcgttttcatattcagaaagaatatctaataagatgtagtttttatgatttgttatacatatgga

SEQ ID NO: 60 - DNA, 750 bp, Artificial Sequence
  Promoter of ctt1 gene
ttaactgcaaagacccgtcacctatgaataagatgactctcttgttggggtcaatctcctcagcggcaaaggcagcacctaaagttgctcctgttgtaaaaccgatagacccccacaacacttgcgagataccgtaggcgtccttaggaaagatagtttgattgataccgaaggcagacgtgccggtctcggaaatgataacatcaccttcttgcaagaacttggacaattcgttccacaaccactcttgtttcaagggcgtgctagcagatacacctttgtttgcgggagttttggttggtacgggaacgctcttgtagcccttaataacatcgggaataaccttcagtaagttttgtagtgcaaatttcatttgtacgccggggaacgtagcgttcttcacctttacgtaatcggaatgaaactccactacatttttagtcttgtaggagtaggaaaacgaacctgtgttaaaatcagagagcaaagcaccgaccgaaaggatcaaatcagccgactcaacggcctgtttcacgtctggtttggacagcgttcccacataaacaccgccatatctgggatgctgttcatctattgaccctttacctaaaggtgtcacaaaagctgggaattgcgtcaaatcaattaacttctgggtttcctttttaacgttgtgcctagaagcacaggcatccgatagtatgacagggtttttcgaattctggatcaattctagtacggtatcaataa

SEQ ID NO: 61 - DNA, 750 bp, Artificial Sequence
  Promoter of ygp1 gene
tctcataattggtcagaagaaaggaaatctaggcaattgagttctttgggtaaaaaagagtcacaatttttaaggctgcgtagaacgagattatcgttagaagatttccacactgtgaaagtcattggaaaaggggcatttggtgaagtcagactggttcagaagaaagacaccggaaaaatatatgcaatgaaaactttattaaaatccgaaatgtacaaaaaggaccaattagcacacgtcaaggctgaaagggatgttctggctggaagtgattctccatgggtggtttcgctatattactcattccaagatgctcaatacttatatttaatcatggaatttttgcccggtggtgatttgatgaccatgctaatcaggtggcaactatttacagaggatgttactagattttacatggctgagtgtattttggccatcgaaaccattcataaattaggattcattcacagagatattaaaccagataatattttgatcgatattagaggtcatataaaattatctgatttcgggttgtctacggggttccataaaactcacgactcaaactactataaaaagcttttgcaacaagatgaggcaactaatggtatttccaagccaggtacttacaatgcaaatacaaccgatactgcaaataaaaggcaaacaatggttgtggattctattagtctaacaatgtcaaacaggcaacaaattcagacatggagaaaatcacgtcgttta

SEQ ID NO: 62 - DNA, 750 bp, Artificial Sequence
  Promoter of gsy2 gene
tattatagctcttgataacaatgaaatttcaacaattattcactcaaccctcaagtgtatatcgccaagatcctaatcacccctctctggtggccttccctcattgtttttacacagaacaagaatccataaaatcaagatattgggaacaattcattgtcttaggaacaagtttttataatttagtttcattagcctattgtaacttcttgtttttttttttattttggctggcaaaacaagaagtataaagaagttaccactgctataacgtatatcttaaatcaagtgggcaattcttcgaataaacttagaggcgctgaagaaagaaccgctcaattagttcgatcatacaaaatacagatattgcgctgtgtattatcattacagaaagtgtgatataaacaatgctgtgttgtattcggtcttttttttccgcctgcagattgtcgtttttgtttttttttctcgtgtagctgcagttatcatcgttttgcatgtctgttctgtcggtgaagccgatctcatggcacttcgctttgataccgtaaagaatcttcttcattaatacggcactgcaaggcgatatctatctctggggttaggactgataaataggtacttatcagttatcactagagacgaactgtttacgttttgaagctcgacagcaggggttcttaacgggcgtcggcctagacctcaccttgcgagaaatttttagttcatatccaggggatggtgaatggt

SEQ ID NO: 63 - DNA, 750 bp, Artificial Sequence
  Promoter of hsp12 gene
gcttgtccacaatcaccatggggttatattgcattatttttttatcttcgttatcattccatactgtggcttcattccagtattttatgagtttgctgaaggattcgggggtaatagtgcccacggtggtcaaaacctttagcgtctcagtgacctgttccaaatccccctcttccattcgccttatataaaatccatcgggtaagctcattattttatgttgatatccagcttccgttcgtcaactattctctcgagctcagttttgggttttggcatgtaaacaatgccttcgcttgtattacgcaaaaaaaaaaaaaataaataaaaaaaaaaaaaaaaaaataaggtataaatcgttggttcttttatgcacaattatttaactatagttatctatttacgtaaaggcttctatttttccttatctacaagaaattgcatgaagtttaattttttttgtcacctttgatcttcctgaatgtgttggtaatgaaattttctagtctcttcaaagtgttgtcgtcatccttttttgcgtgtttttcctcttcttgtttttgtttcttcagtatctcttcattaatggctgtacccgcggctgtgcccggttgcatggttttcttggccatgtcatcgttggtgacatctgccagtgcctcccacaagcatttttctgccagcgattgcggtttcttaacaacgatcttgtactggactttcatatcaccgcgaatggttgatgtttta

SEQ ID NO: 64 - DNA, 750 bp, Artificial Sequence
  Promoter of mol1/thi4 genes
tcgttgaaagttctagatttgggtacggaagcagccatgtaaatggtcttcctttgtaataatatcgtcactggagtttttaaattagagtacggattcaagttaacgaaaaatttctgttggtatctgatagcaatttgtcgacaaggtggagagtttttgaagtttttttttgtaacagttatgttacactatttaccagagaaacaccaagacaggagccctcagaagctttcgtgttgtcagaacataaatatcttctatatgttttattcttttatattaatcaatattttatgttaaatcatattattattcgttaatgcttaacttgccctatttcaaaatcggaagggtagtatggtgacaagcggtgtttttggtgtatgtttactgctaagagtataccgtcctgtttacttttttaccttttgttccgttattgatttttcgcagcaaggttgaagcttccatatttgaaaatgaaattaagaagtagagatgaaaaacagcgacgaaattactttttatagcttctttcccttctgactccgttttataaacaggggatctttaagggcttacaaaataaatataaacactaacacatatatattattcgacctacagtttttgcgaccaatgaagtttacctattttgagatggtgcttacgtagtttgcttaaggtcaaggcgggattaagcttgcgctttctctaaataatatcatatgttacagacagggataa

SEQ ID NO: 65 - protein, 695 aa, Geobacillus stearothermophilus
MYNPNAAEASSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 66 - protein, 411 aa, Citrobacter braakii
EEQNGMKLERVVIVSRHGVRAPTKFTPIMKDVTPDQWPQWDVPLGWLTPRGGELVSELGQYQRLWFTSKGLLNNQTCPSPGQVAVIADTDQRTRKTGEAFLAGLAPKCQIQVHYQKDEEKNDPLFNPVKMGKCSFNTLKVKNAILERAGGNIELYTQRYQSSFRTLENVLNFSQSETCKTTEKSTKCTLPEALPSEFKVTPDNVSLPGAWSLSSTLTEIFLLQEAQGMPQVAWGRITGEKEWRDLLSLHNAQFDLLQRTPEVARSRATPLLDMIDTALLTNGTTENRYGIKLPVSLLFIAGHDTNLANLSGALDLKWSLPGQPDNTPPGGELVFEKWKRTSDNTDWVQVSFVYQTLRDMRDIQPLSLEKPAGKVDLKLIACEEKNSQGMCSLKSFSRLIKEIRVPECAVTE

SEQ ID NO: 67 - protein, 410 aa, Escherichia coli
QSEPELKLESVVIVSRHGVRAPTKATQLMQDVTPDAWPTWPVKLGWLTPRGGELIAYLGHYQRQRLVADGLLAKKGCPQSGQVAIIADVDERTRKTGEAFAAGLAPDCAITVHTQADTSSPDPLFNPLKTGVCQLDNANVTDAILSRAGGSIADFTGHRQTAFRELERVLNFPQSNLCLKREKQDESCSLTQALPSELKVSADNVSLTGAVSLASMLTEIFLLQQAQGMPEPGWGRITDSHQWNTLLSLHNAQFYLLQRTPEVARSRATPLLDLIKTALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 68 - protein, 19 aa, Saccharomyces cerevisiae
MLLQAFLFLLAGFAAKISA

SEQ ID NO: 69 - protein, 18 aa, Saccharomyces cerevisiae
MQLLRCFSIFSVIASVLA

SEQ ID NO: 70 - DNA, 500 bp, Saccharomyces cerevisiae
tagcgtgttacgcacccaaactttttatgaaagtctttgtttataatgatgaggtttataaatatatagtggagcaaagattaatcactaaatcaagaagcagtaccagtattttttttatatcaagtagtgataatggaaatagcccaaatttggcttccgtcggcacatagcacgtttgagagacattatcaccatcaagcatcgagccgcccaaacctaactgtataagttttttcacgtttttgatttttccttgcacacttcgatattactctcacgataaaagggccgaagagaatatttttcttgaacatccagaattttaattcggagaaatttcacaagccgccgatttaagggtcctgtgttcttaataatcagcctctctcaaagcaggtaagaggcagtctttcttttaacaataggagacattcgaactaaaacatcagccccaaaaatgcgcttgaaggtcattaggatttggatttcttcctcat

SEQ ID NO: 71 - protein, 460 aa, Pyrococcus furiosus
MNIKKLTPLLTLLLFFIVLASPVSAAKYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 72 - protein, 457 aa, Thermococcus hydrothermalis
MARKVLVALLVFLVVLSVSAVPAKAETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVG

SEQ ID NO: 73 - DNA, 390 bp, Artificial Sequence
  SPI1 tethering moiety
  CDS (1)..(390)
ttggtatctaattctagttcctctgtaatcgtggtaccatcaagcgatgctactattgccggtaacgatacagccacgccagcaccagagccatcatccgccgctccaatattctacaactcgactgctactgcaacacagtacgaagttgtcagtgaattcactacttactgcccagaaccaacgactttcgtaacgaatggcgctacattcactgttactgccccaactacgttaacaattaccaactgtccttgcactatcgagaagcctacttcagaaacatcggtttcttctacacatgatgtggagacaaattctaatgctgctaacgcaagagcaatcccaggagccctaggtttggctggtgcagttatgatgcttttatga

SEQ ID NO: 74 - protein, 129 aa, Artificial Sequence
  Synthetic Construct
LVSNSSSSVIVVPSSDATIAGNDTATPAPEPSSAAPIFYNSTATATQYEVVSEFTTYCPEPTTFVTNGATFTVTAPTTLTITNCPCTIEKPTSETSVSSTHDVETNSNAANARAIPGALGLAGAVMMLL

SEQ ID NO: 75 - DNA, 66 bp, Artificial Sequence
  21aa-truncation of the SPI1 tethering moiety
  CDS (1)..(66)
gctgctaacgcaagagcaatcccaggagccctaggtttggctggtgcagttatgatgcttttatga

SEQ ID NO: 76 - protein, 21 aa, Artificial Sequence
  Synthetic Construct
AANARAIPGALGLAGAVMMLL

SEQ ID NO: 77 - DNA, 156 bp, Artificial Sequence
  51aa-truncation of the SPI1 tethering moiety
  CDS (1)..(156)
ttaacaattaccaactgtccttgcactatcgagaagcctacttcagaaacatcggtttcttctacacatgatgtggagacaaattctaatgctgctaacgcaagagcaatcccaggagccctaggtttggctggtgcagttatgatgcttttatga

SEQ ID NO: 78 - protein, 51 aa, Artificial Sequence
  Synthetic Construct
LTITNCPCTIEKPTSETSVSSTHDVETNSNAANARAIPGALGLAGAVMMLL

SEQ ID NO: 79 - DNA, 246 bp, Artificial Sequence
  81aa-truncation of the SPI1 tethering moiety
  CDS (1)..(246)
gaagttgtcagtgaattcactacttactgcccagaaccaacgactttcgtaacgaatggcgctacattcactgttactgccccaactacgttaacaattaccaactgtccttgcactatcgagaagcctacttcagaaacatcggtttcttctacacatgatgtggagacaaattctaatgctgctaacgcaagagcaatcccaggagccctaggtttggctggtgcagttatgatgcttttatga

SEQ ID NO: 80 - protein, 81 aa, Artificial Sequence
  Synthetic Construct
EVVSEFTTYCPEPTTFVTNGATFTVTAPTTLTITNCPCTIEKPTSETSVSSTHDVETNSNAANARAIPGALGLAGAVMMLL

SEQ ID NO: 81 - DNA, 336 bp, Artificial Sequence
  111aa-truncation of the SPI1 tethering moiety
  CDS (1)..(336)
attgccggtaacgatacagccacgccagcaccagagccatcatccgccgctccaatattctacaactcgactgctactgcaacacagtacgaagttgtcagtgaattcactacttactgcccagaaccaacgactttcgtaacgaatggcgctacattcactgttactgccccaactacgttaacaattaccaactgtccttgcactatcgagaagcctacttcagaaacatcggtttcttctacacatgatgtggagacaaattctaatgctgctaacgcaagagcaatcccaggagccctaggtttggctggtgcagttatgatgcttttatga

SEQ ID NO: 82 - protein, 111 aa, Artificial Sequence
  Synthetic Construct
IAGNDTATPAPEPSSAAPIFYNSTATATQYEVVSEFTTYCPEPTTFVTNGATFTVTAPTTLTITNCPCTIEKPTSETSVSSTHDVETNSNAANARAIPGALGLAGAVMMLL

SEQ ID NO: 83 - DNA, 339 bp, Artificial Sequence
  CCW12 tethering moiety
  CDS (1)..(339)
gttaccactgctactgtcagccaagaatctaccactttggtcaccatcacttcttgtgaagaccacgtctgttctgaaactgtctccccagctttggtttccaccgctaccgtcaccgtcgatgacgttatcactcaatacaccacctggtgcccattgaccactgaagccccaaagaacggtacttctactgctgctccagttacctctactgaagctccaaagaacaccacctctgctgctccaactcactctgtcacctcttacactggtgctgctgctaaggctttgccagctgctggtgctttgttggctggtgccgctgctttgttgttgtaa

SEQ ID NO: 84 - protein, 112 aa, Artificial Sequence
  Synthetic Construct
VTTATVSQESTTLVTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 85 - DNA, 75 bp, Artificial Sequence
  24aa-truncation of the CCW12 tethering moiety
  CDS (1)..(75)
tacactggtgctgctgctaaggctttgccagctgctggtgctttgttggctggtgccgctgctttgttgttgtaa

SEQ ID NO: 86 - protein, 24 aa, Artificial Sequence
  Synthetic Construct
YTGAAAKALPAAGALLAGAAALLL

SEQ ID NO: 87 - DNA, 150 bp, Artificial Sequence
  49aa-truncation of the CCW12 tethering moiety
  CDS (1)..(150)
actgctgctccagttacctctactgaagctccaaagaacaccacctctgctgctccaactcactctgtcacctcttacactggtgctgctgctaaggctttgccagctgctggtgctttgttggctggtgccgctgctttgttgttgtaa

SEQ ID NO: 88 - protein, 49 aa, Artificial Sequence
  Synthetic Construct
TAAPVTSTEAPKNTTSAAPTHSVTSYTGAAAKALPAAGALLAGAAALLL

SEQ ID NO: 89 - DNA, 225 bp, Artificial Sequence
  74aa-truncation of the CCW12 tethering moiety
  CDS (1)..(225)
accgtcgatgacgttatcactcaatacaccacctggtgcccattgaccactgaagccccaaagaacggtacttctactgctgctccagttacctctactgaagctccaaagaacaccacctctgctgctccaactcactctgtcacctcttacactggtgctgctgctaaggctttgccagctgctggtgctttgttggctggtgccgctgctttgttgttgtaa

SEQ ID NO: 90 - protein, 74 aa, Artificial Sequence
  Synthetic Construct
TVDDVITQYTTWCPLTTEAPKNGTSTAAPVTSTEAPKNTTSAAPTHSVTSYTGAAAKALPAAGALLAGAAALLL

SEQ ID NO: 91 - DNA, 300 bp, Artificial Sequence
  99aa-truncation of the CCW12 tethering moiety
  CDS (1)..(300)
gtcaccatcacttcttgtgaagaccacgtctgttctgaaactgtctccccagctttggtttccaccgctaccgtcaccgtcgatgacgttatcactcaatacaccacctggtgcccattgaccactgaagccccaaagaacggtacttctactgctgctccagttacctctactgaagctccaaagaacaccacctctgctgctccaactcactctgtcacctcttacactggtgctgctgctaaggctttgccagctgctggtgctttgttggctggtgccgctgctttgttgttgtaa

SEQ ID NO: 92 - protein, 99 aa, Artificial Sequence
  Synthetic Construct
VTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 93 - protein, 15 aa, Artificial Sequence
  Linker 1
GGGGSGGGGSGGGGS

SEQ ID NO: 94 - protein, 8 aa, Artificial Sequence
  Linker 2
GGGGGGGG

SEQ ID NO: 95 - protein, 40 aa, Artificial Sequence
  Linker 3
GGGGSGGGGSGGGGSGGGGSGGGGSGGGGSGGGGSGGGGS

SEQ ID NO: 96 - protein, 12 aa, Artificial Sequence
  Linker 4
GSAGSAAGSGEF

SEQ ID NO: 97 - protein, 12 aa, Artificial Sequence
  Linker 5
EAAKEAAKEAAK

SEQ ID NO: 98 - protein, 20 aa, Artificial Sequence
  Linker 6
APAPAPAPAPAPAPAPAPAP

SEQ ID NO: 99 - protein, 46 aa, Artificial Sequence
  Linker 7
AEAAAKEAAAKEAAAKEAAAKALEAEAAAKEAAAKEAAAKEAAAKA

SEQ ID NO: 100 - protein, 4 aa, Artificial Sequence
  EA2K motif
EAAK

SEQ ID NO: 101 - protein, 5 aa, Artificial Sequence
  EA3K motif
EAAAK

SEQ ID NO: 102 - DNA, 1752 bp, Aspergillus niger
tccaatggtattgaggcttccttgttgactgatccaaaagatgtttctggtagaaccgtcgattacattattgctggtggtggtttgactggtttaactactgctgctagattgactgaaaacccaaacatctccgttttggttatcgaatctggttcctacgaatctgatagaggtccaattatcgaagatttgaacgcctacggtgatatctttggttcttctgttgatcatgcttacgaaaccgttgaattggctaccaacaatcaaactgccttgattagatctggtaacggtttaggtggttctactttggttaatggtggtacttggactagaccacataaggctcaagttgattcttgggaaactgtttttggtaacgaaggttggaattgggataatgttgctgcttattcattgcaagctgaaagggctagagcaccaaatgctaaacaaattgctgctggtcattacttcaatgcctcttgtcatggtgttaacggtactgttcatgctggtccaagagatactggtgatgattattctccaatcgttaaggctttgatgtccgctgttgaagatagaggtgttccaactaagaaagatttcggttgtggtgatccacatggtgtttctatgtttccaaataccttgcacgaagatcaagttagatctgatgctgcaagagaatggttgttgccaaattatcaaagaccaaacttgcaggtcttgactggtcaatatgttggtaaggttttgttgtcccaaaacggtactactccaagagctgttggtgttgaatttggtactcataagggtaacacccataacgtttacgccaaacatgaagttttgttagctgctggttctgctgtttctccaactattttggaatactccggtatcggtatgaagtccattttggaaccattgggtattgataccgttgttgatttgccagttggtttgaacttacaagatcaaactaccgctaccgtcagatctagaattacttctgctggtgctggtcaaggtcaagctgcttggtttgctacttttaacgaaactttcggtgactactctgaaaaggcccatgaattattgaacaccaaattggaacaatgggctgaagaggctgttgctagaggtggttttcataacactactgccttgttgatccagtacgaaaattacagagattggatcgttaaccacaacgttgcttactctgagttgtttttggatactgctggtgttgcttcttttgatgtatgggatttgttgccattcaccagaggttacgttcacattttggataaggatccatacttgcatcatttcgcttacgacccacaatactttttgaacgaattggacttgttgggtcaagccgctgctactcaattggctagaaacatttctaattccggtgctatgcaaacctatttcgctggtgaaactattccaggtgataacttggcttatgatgctgatttgtctgcttggactgagtacattccataccatttcagaccaaattaccacggtgttggtacttgttctatgatgccaaaagaaatgggtggtgttgttgataatgcagctagagtttacggtgttcaaggtttgagagttatcgatggttctattccaccaactcaaatgtcctctcatgttatgactgttttctacgctatggccttgaagatttccgatgcaattttggaagattacgcctccatgcaataa

SEQ ID NO: 103 - protein, 583 aa, Aspergillus niger
SNGIEASLLTDPKDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGVNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAKHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQ

SEQ ID NO: 104 - DNA, 1437 bp, Aspergillus oryzae
gctactccagctgattggagatcacaatctatctactttttgttgaccgacagattcgctagaactgatggttctactactgctacttgtaataccgctgatagaaagtattgtggtggtacttggcaaggtatcatcgataagttggattacattcaaggtatgggtttcaccgctatttggattactccagttactgctcaattgccacaaactactgcttatggtgatgcttatcatggttattggcaacaggatatctactccttgaacgaaaattacggtactgccgatgatttgaaggctttgtcatctgctttacatgaacgtggtatgtacttgatggttgatgttgttgctaaccacatgggttatgatggtgctggttcttctgttgattactctgtttttaagcccttcagctcccaagattactttcatccattctgcttgatccaaaactacgaagatcaaactcaagtcgaagattgctggttgggtgataatactgtttctttgccagatttggataccaccaaggatgttgttaagaacgaatggtatgattgggtcggttctttggtttccaactactctattgatggtttgagaatcgataccgtcaagcacgttcaaaaagatttttggccaggttacaacaaagctgctggtgtttactgtattggtgaagttttagatggtgatccagcttacacttgtccataccaaaatgttatggatggcgttttgaactacccaatctactacccattattgaacgctttcaagtctacctctggttctatggatgacttgtacaacatgatcaacaccgttaagtctgattgtccagattctactttgttgggtactttcgttgaaaaccacgataatccaagattcgcttcttacaccaacgatattgctttggctaaaaacgttgccgccttcattattttgaacgatggtattccaattatctacgccggtcaagaacaacattatgctggtggtaatgatccagcaaatagagaagctacttggttgtctggttatccaactgattccgagttgtacaaattgattgcttccgctaacgccattagaaactacgctatttctaaggatactggcttcgttacttacaagaattggcctatctacaaggatgataccactattgctatgagaaagggtacagatggttctcaaatcgttaccatcttgtctaacaaaggtgcttctggtgattcctacactttgtctttgtctggtgcaggttatactgctggtcaacaattgactgaagttattggttgtactaccgttaccgttggttctgatggtaatgttcctgttccaatggctggtggtttgccaagagtcttgtatccaacagaaaaattggccggttccaagatctgttcttcttcttga

SEQ ID NO: 105 - protein, 478 aa, Aspergillus oryzae
ATPADWRSQSIYFLLTDRFARTDGSTTATCNTADRKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCLIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

SEQ ID NO: 106 - DNA, 63 bp, Artificial Sequence
  Signal peptide from fungal amylase from Aspergillus oryzae
atgatggttgcttggtggtctttgttcttgtacggtttacaagttgctgctccagctttggct

SEQ ID NO: 107 - protein, 21 aa, Artificial Sequence
  Signal peptide from fungal amylase from Aspergillus oryzae
MMVAWWSLFLYGLQVAAPALA

SEQ ID NO: 108 - protein, 687 aa, Geobacillus stearothermophilus
MSSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

(19) United States
(12) Patent Application Publication
(10) Pub. No.: US 2022/0127564 A1
(43) Pub. Date: Apr. 28, 2022

(54) RECOMBINANT YEAST HOST CELLS EXPRESSING CELL-ASSOCIATED HETEROLOGOUS PROTEINS

(71) Applicant: Lallemand Hungary Liquidity Management LLC, Budapest (HU)

(72) Inventors: Aaron Argyros, Lebanon, NH (US); Michelle Oeser, Croydon, NH (US); Erin Wiswall, Danbury, NH (US); Janet Fisher, Enfield, NH (US); Johannes Van Eijk, Longueuil (CA); J. Kevin Kraus, Tenafly, NJ (US); Kevin Wenger, Hanover, NH (US); Brooks Henningsen, Salisbury, NH (US); Ryan Skinner, South Royalton, VT (US)

(21) Appl. No.: 16/493,595

(22) PCT Filed: Mar. 13, 2018
(86) PCT No.: PCT/IB2018/051671

§ 371 Date: Sep. 12, 2019

Related U.S. Application Data
(60) Provisional application No. 62/470,631, filed on Mar. 13, 2017.
(60) Provisional application No. 62/625,777, filed on Feb. 2, 2018.

(51) Int. Cl. C12N 1/18; C12P 21/02; C12N 1/06; C12N 9/00

(52) U.S. Cl. CPC C12N 1/18; C12N 9/00; C12N 1/063; C12P 21/02

(57) ABSTRACT

The present disclosure concerns recombinant yeast host cells expressing cell-associated heterologous proteins which are expressed during the propagation phase of the recombinant yeast host cells and processes for propagating same. The recombinant yeast host cells can be 5 used to make a yeast composition or a yeast product enriched in the heterologous proteins.

### STATEMENT REGARDING SEQUENCE LISTING

The sequence listing associated with this application is provided in text format in lieu of a paper copy and is hereby incorporated by reference into the specification. The name of the text file containing the sequence listing is PCT-Sequence listing as filed. The text file is 224 KB, was created on Mar. 13, 2018 and is being submitted electronically.

### TECHNOLOGICAL FIELD

The present disclosure relates to recombinant yeast host cells expressing cell-associated heterologous polypeptides (including heterologous enzymes), compositions comprising the same as well as processes including them.

### BACKGROUND

Commercial enzyme production using sensitive bacteria and fungi require long fermentations, specialized media and sterile conditions. Because the enzymes are excreted, the large volume of liquid broth must be separated from the biomass and then concentrated and purified to recover the enzyme. Yeast are generally more robust than bacteria and fungi and can be grown more quickly on less expensive media under less than sterile conditions.

Yeast are also used in various industrial processes for making yeast compositions and products. In such processes, it is common to supplement yeasts with purified exogenous proteins (which may be heterologous to the yeasts) to obtain more rapidly or more efficiently the yeasts-containing or yeast-derived products. However, the costs of the adding such exogenous proteins may be significant and there is an incentive to lower the utilization or render obsolete the use of exogenous proteins in the production of yeast products.

There is thus a need to be provided with heterologous proteins which can be obtained in a sufficient amount to be used in a subsequent commercial process and at a costs which would allow the use of the heterologous proteins on a commercial scale.

### BRIEF SUMMARY

The present disclosure relates to recombinant yeast host cells that express and remain associated with heterologous proteins while the yeast host cells are being propagated, processes for propagating same as well as for making yeast compositions and yeast products from same.

According to a first aspect, the present disclosure relates to a process for making a cell-associated heterologous protein from a recombinant yeast host cell. The recombinant yeast host cell has an heterologous nucleic acid molecule encoding the cell-associated heterologous protein and the heterologous nucleic acid molecule is operatively associated with an heterologous promoter allowing the expression of the heterologous nucleic acid molecule during propagation. The process comprises propagating the recombinant yeast host cell in a medium placed in a vessel according to a baker's yeast production method so as to allow the expression of the cell-associated heterologous protein. In an embodiment, the baker's yeast production method is a continuous method or a fed batch method. In yet another embodiment, the specific growth rate of the recombinant yeast host cell during the propagation is 0.25 h⁻¹ or less. In yet a further embodiment, the process further comprises controlling the aeration rate of the vessel is at least about 0.5 or about 1.0 air volume/vessel volume/minute. In still a further embodiment, the medium comprises a carbohydrate source, a nitrogen source, a phosphorous source and optionally micronutrients. In an embodiment, the carbohydrate source is derived from molasses, corn, glycerol and/or a lignocellulosic biomass. In a further embodiment, the nitrogen source is ammonia. In still a further embodiment, the phosphorous source is phosphoric acid. In another embodiment, the further comprises controlling the addition of the carbohydrate source to the medium so as to limit the growth rate of the recombinant yeast host cell. In still a further embodiment, the process comprises maintaining the concentration of the carbohydrate source at 0.1 or 0.0001 weight percentage or less with respect to the total volume of the medium. In still another embodiment, the process further comprises adding the nitrogen source and/or the phosphorous source to match the growth rate of the recombinant yeast host cell. In yet another embodiment, the process further comprises controlling the pH of the medium between about 4.0 and 5.0, for example at about 4.5. In still another embodiment, the process further comprises controlling the temperature of the medium between about 20° C. to about 40° C., for example at between about 30° C. to about 35° C. In an embodiment, after the propagation step, the concentration of the recombinant yeast host cell is of at least 0.25 or 1 weight % of the total volume of the medium. In an embodiment, the recombinant yeast host cell is from the genus Saccharomyces sp. In a further embodiment, the recombinant yeast host cell is from the species Saccharomyces cerevisiae.

According to a second aspect, the present disclosure relates to a recombinant yeast host cell for making a yeast composition or a yeast product, the recombinant yeast host cell having an heterologous nucleic acid molecule encoding a cell-associated heterologous protein, wherein the heterologous nucleic acid molecule is operatively associated with an heterologous promoter allowing the expression of the heterologous nucleic acid molecule during propagation. In an embodiment, the cell-associated heterologous protein represents at least 0.1% (in dry weight percent) of the total proteins in the yeast composition or in the yeast product. In an embodiment, the cell-associated heterologous polypeptide is an heterologous enzyme, such as, for example, an oxidoreductase, a transferase, an hydrolase, a lyase, an isomerase, a phosphatase and/or a ligase. In an embodiment, the heterologous enzyme is the glycosylase such as, for example, an heterologous amylase. In embodiments in which the cell-associated heterologous protein is the amylase, it can be, without limitation, a maltogenic alpha-amylase, a glucoamylase, an alpha-amylase or a fungal amylase. In an embodiment the amylase is the maltogenic amylase. In an embodiment the amylase is the glucoamylase. In an embodiment, the amylase is the alpha-amylase. In an embodiment, the amylase is a fungal amylase. In still a further embodiment, the oxidase is a glucose oxidase. In still another embodiment, the heterologous enzyme is a phosphatase, such as, for example, a phytase. In a further embodiment, the heterologous enzyme is an oxidase, such as, for example, a glucose oxidase. In another embodiment, the heterologous nucleic acid molecule allows the intracellular expression of the heterologous cell-associated protein. In still another embodiment, the heterologous nucleic acid molecule allows the expression of a membrane-associated heterologous protein. In a further embodiment, the heterologous nucleic acid molecule allows the expression of a tethered heterologous protein. In yet a further embodiment, the tethered heterologous protein is a chimeric protein of formula (I):

(NH₂)HP-L-TT(COOH) (I)

wherein HP is the heterologous polypeptide, L is present or absent and is an amino acid linker and TT is an amino acid tethering moiety for associating the heterologous polypeptide to a cell wall of the recombinant yeast host cell and “-” is an amide linkage. In the chimeric protein of formula (I), (NH₂) indicates the location of the amino terminus of the chimeric protein whereas (COOH) indicates the carboxyl terminus of the chimeric protein. In another embodiment, the tethered heterologous protein is a chimeric protein of formula (II):

(NH₂)TT-L-HP(COOH) (II)

wherein HP is the heterologous polypeptide, L is present or absent and is an amino acid linker, TT is an amino acid tethering moiety for associating the heterologous polypeptide to a cell wall of the recombinant yeast host cell and “-” is an amide linkage. In the chimeric protein of formula (II), (NH₂) indicates the location of the amino terminus of the chimeric protein whereas (COOH) indicates the carboxyl terminus of the chimeric protein. In an embodiment of the chimeric protein, L is present and can, for example, comprises one or more G₄S (SEQ ID NO: 41) motifs and/or one or more EA₂K (SEQ ID NO: 100) or EA₃K (SEQ ID NO: 101) motifs. In other embodiments of the chimeric protein, TT can comprise a transmembrane domain, a variant or a fragment thereof. For example, TT can be from a FLO1 protein. For example, TT can have the amino acid sequence of SEQ ID NO: 14, be a variant of the amino acid sequence of SEQ ID NO: 14 or be a fragment of the amino acid sequence SEQ ID NO: 14. In further embodiments of the chimeric protein, TT can be modified by a post-translation mechanism to have a glycosylphosphatidylinositol (GPI) anchor. For example, TT can from a SED1 protein, a TIR1 protein, a CWP2 protein, a CCW12 protein, a SPI1 protein, a PST1 protein or a combination of a AGA1 protein and a AGA2 protein. In a specific embodiment, TT is from the SPI1 protein and can have, for example, the amino acid sequence of SEQ ID NO: 74, be a variant of the amino acid sequence of SEQ ID NO: 74 or be a fragment of the amino acid sequence SEQ ID NO: 74. In a further embodiment, TT can be a fragment of the SPI protein an can have the amino acid sequence of SEQ ID NO: 76, 78, 80 or 82; be a variant of the amino acid sequence of SEQ ID NO: 76, 78, 80 or 82 or be a fragment of the amino acid sequence of SEQ ID NO: 76, 78, 80 or 82. In another specific embodiment, TT can be from the CCW12 protein and can, for example, have the amino acid sequence of SEQ ID NO: 84, be a variant of the amino acid sequence of SEQ ID NO: 84 or be a fragment of the amino acid sequence of SEQ ID NO: 84. In yet a further embodiment, TT can be a fragment of the CCW12 protein and can have the amino acid sequence of SEQ ID NO: 86, 88, 90 or 92; be a variant of the amino acid sequence of SEQ ID NO: 86, 88, 90 or 92 or be a fragment of the amino acid sequence of SEQ ID NO: 86, 88, 90 or 92. In another embodiment, TT is from the combination of the AGA1 protein and the AGA2 protein. In yet another embodiment, the combination of the AGA1 protein and the AGA2 protein has the amino acid sequence of SEQ ID NO: 24, is a variant of the amino acid sequence of SEQ ID NO: 24, is a fragment of the amino acid sequence of SEQ ID NO: 24, has the amino acid sequence of SEQ ID NO: 26, is a variant of the amino acid sequence of SEQ ID NO: 26 or is a fragment of the amino acid sequence of SEQ ID NO: 26. In an additional embodiment, the promoter is a native or an heterologous promoter such as, for example comprises the promoter from the tdh1 gene, the hor7 gene, the hsp150 gene, the hxt7 gene, the gpm1 gene, the pgk1 gene and/or the stl1 gene. In an embodiment, the heterologous promoter comprises the promoter from the tdh1 gene. In still another embodiment, In an embodiment, the heterologous promoter comprises the promoter from the hor7 gene. In yet another embodiment, the heterologous nucleic acid molecule is operatively associated with a terminator. In yet a further embodiment, the terminator is a native or an heterologous terminator and can comprise, for example, the terminator from the dit1 gene, the idp1 gene, the gpm1 gene, the pma1 gene, the tdh3 gene, the hxt2 gene and/or the ira2 gene. In a specific embodiment, the heterologous terminator can be from the dit1 gene. In another specific embodiment, the heterologous terminator can be from the adh3 gene. In yet another specific embodiment, the heterologous terminator can be from the idp1 gene. In still another embodiment, the heterologous nucleic acid molecule encoding the membrane-associated heterologous polypeptide is associated with a further nucleic acid molecule encoding an heterologous signal peptide. In an embodiment, the heterologous signal peptide is derived from a prokaryotic protein, such as, for example, a bacterial protein. In a further embodiment, the heterologous signal peptide can be from an invertase protein (having the amino acid sequence of SEQ ID NO: 68, being a variant of the amino acid sequence of SEQ ID NO: 68 or being a fragment of the amino acid sequence of SEQ ID NO: 68), an AGA2 protein (having the amino acid sequence of SEQ ID NO: 69, being a variant of the amino acid sequence of SEQ ID NO: 69 or being a fragment of the amino acid sequence of SEQ ID NO: 69) or a fungal amylase (having the amino acid sequence of SEQ ID NO: 107, being a variant of the amino acid sequence of SEQ ID NO: 107 or being a fragment of the amino acid sequence of SEQ ID NO: 107). In an embodiment, the recombinant yeast host cell is from the genus Saccharomyces sp. In a further embodiment, the recombinant yeast host cell is from the species Saccharomyces cerevisiae.

According to a third aspect, the present disclosure provides a process for making a yeast composition. Broadly, the process comprises a) propagating the recombinant yeast host cell having a cell-associated heterologous protein as defined herein to obtain a propagated recombinant yeast host cell and b) formulating the propagated yeast host cell into the yeast composition. In an embodiment, the yeast composition is a cream. In still another embodiment, step a) is conducted in a culture medium which can, for example, comprises molasses. In an embodiment, the cell-associated heterologous protein represents at least 0.1% (in dry weight percent) of the total proteins in the yeast composition. In yet another embodiment, the cell-associated heterologous protein is an heterologous enzyme. The process can comprise propagating the recombinant yeast host cell in a medium placed in a vessel according to a baker's yeast production method so as to allow the expression of the cell-associated heterologous protein. In an embodiment, the baker's yeast production method is a continuous method or a fed batch method. In yet another embodiment, the specific growth rate of the recombinant yeast host cell during the propagation is 0.25 h⁻¹ or less. In yet a further embodiment, the process further comprises controlling the aeration rate of the vessel is at least about 0.5 or about 1.0 air volume/vessel volume/minute. In still a further embodiment, the medium comprises a carbohydrate source, a nitrogen source, a phosphorous source and optionally micronutrients. In an embodiment, the carbohydrate source is derived from molasses, corn, glycerol and/or a lignocellulosic biomass. In a further embodiment, the nitrogen source is ammonia. In still a further embodiment, the phosphorous source is phosphoric acid. In another embodiment, the further comprises controlling the addition of the carbohydrate source to the medium so as to limit the growth rate of the recombinant yeast host cell. In still a further embodiment, the process comprises maintaining the concentration of the carbohydrate source at 0.1 or 0.0001 weight percentage or less with respect to the total volume of the medium. In still another embodiment, the process further comprises adding the nitrogen source and/or the phosphorous source to match the growth rate of the recombinant yeast host cell. In yet another embodiment, the process further comprises controlling the pH of the medium between about 4.0 and 5.0, for example at about 4.5. In still another embodiment, the process further comprises controlling the temperature of the medium between about 20° C. to about 40° C., for example at between about 30° C. to about 35° C. In an embodiment, after the propagation step, the concentration of the recombinant yeast host cell is of at least 0.25 or 1 weight % of the total volume of the medium. In an embodiment, the recombinant yeast host cell is from the genus Saccharomyces sp. In a further embodiment, the recombinant yeast host cell is from the species Saccharomyces cerevisiae.

According to a fourth aspect, the present disclosure provides a yeast composition comprising the propagated yeast host cell obtainable or obtained by the process described herein. In an embodiment, the cell-associated heterologous protein represents at least 0.1% (in dry weight percent) of the total proteins in the yeast composition. In yet another embodiment, the cell-associated heterologous protein is an heterologous enzyme.

According to a fifth aspect, the present disclosure provides process for making a yeast product. Broadly, the process comprises a) providing the propagated recombinant yeast host cell obtainable by the process described herein or the yeast composition comprising the propagated recombinant yeast host cell described herein; b) lysing the propagated yeast host cell to obtain a lysed recombinant yeast host cell, c) optionally drying the lysed recombinant yeast host cell to obtain a dried recombinant yeast host cell and d) formulating the lysed recombinant yeast host cell or the dried recombinant yeast host cell to into the yeast product. In an embodiment, step b) comprises submitting the propagated recombinant yeast host cell to autolysis to obtain the lysed recombinant yeast host cell. In an embodiment, step c) is conducted directly after step b) to provide an autolysate as the yeast product. In another embodiment, the lysed recombinant yeast host cell comprises a soluble fraction and an insoluble fraction and the process further comprises, after step b) and prior to step c), separating (for example by filtering) the soluble fraction from the insoluble fraction. In still another embodiment, the process comprising submitting the insoluble fraction to step d) to provide yeast cell walls as the yeast product. In yet a further embodiment, the process comprises submitting the soluble fraction to step d) to provide a yeast extract as the yeast product. In a further embodiment, the process further comprises removing components having a molecular weight equal to or less than about 10 kDa from the soluble fraction to provide a retentate. In yet another embodiment, the process comprises submitting the retentate to step c) to provide a dry retentate as the yeast product. In an embodiment, the cell-associated heterologous protein represents at least 0.1% (in dry weight percent) of the total proteins in the yeast product. In still another embodiment, the further comprises substantially purifying the heterologous protein from the propagated yeast host cell to provide a purified heterologous protein as the yeast product.

According to a sixth aspect, the present disclosure provides a yeast product obtainable or obtained by the process described herein. In an embodiment, the cell-associated heterologous protein represents at least 0.1% (in dry weight percent) of the total proteins in the yeast product. In still another embodiment, the yeast product is provided as an active, a semi-active, an inactive form or a combination thereof.

According to a seventh aspect, the present disclosure provides an isolated heterologous protein obtainable or obtained by the process of described herein. The isolated heterologous protein is produced by a recombinant yeast host cell having an heterologous nucleic acid molecule and a further nucleic acid molecule as defined herein. In addition, the further nucleic acid molecule encodes an heterologous (e.g., bacterial) signal peptide.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 provides the maltogenic amylase (MAA) enzyme activity measured in yeast cell pellets of wild-type (M10474) or recombinant yeast host cells. Results are shown as the maltogenic amylase activity (provided as MANU/mL) in function of type of yeast tested (from left to right, M10474, T2986, T2987, T2988, T2989, T2990, T2991, T2944; strains are described in Table 1).

FIG. 2 provides the glucoamylase enzyme activity measured in pellets (“bound”, light gray) and supernatant (“free”, dark gray) of cultured recombinant yeast host cells expressing an heterologous glucoamylase in the absence (strain M8498) and in the presence (strain M14244) of a Sed1 tether. Results are shown as glucoamylase activity in function of strain used.

FIG. 3 provides alpha-amylase enzyme activity measured in pellets (“bound”, light gray) and supernatant (“free”, dark grey) of cultured recombinant yeast host cells expressing an heterologous alpha-amylase in the presence of a Sed1 tether and a linker (strain M14253), in the presence of a Sed1 tether but no linker (M14254) and in the absence of a Sed1 tether (strain M10074). Results are shown as alpha-amylase activity in function of strain used.

FIG. 4 provides wheat starch activity of various strains expressing a maltogenic amylase. Results are shown as wheat starch MANU per mL (measured at OD 600 nm) for the whole culture (left bars), supernatant (middle bars) and washed pellet (right bars) of the M10474, M13822, M13819, M13879 and T3892 strains (described in Table 1). Data for “M” strains are the average of duplicate cultures. Data for T3892 include the average activity across cultures of eight transformations isolates and the activity of the top performing isolate (□=top isolate, whole culture; Δ=top isolate, supernatant; ∘=top isolate, washed cell pellet). Graphics below indicate the predicted enzyme localization phenotype of each engineering strategy.

FIGS. 5A and 5B provide the phytase activity in culture supernatant (gray bars) or associated with cells (diagonally hatched bars in FIG. 5A or □ in FIG. 5B) for strains expressing free or tethered Citrobacter braakii phytase. Supernatant was incubated with 5 mM sodium phytate solution pH 5.5 for 30 minutes and cells were incubated in the same solution for 2 hours. (FIG. 5A) Absorbance at 700 nm was compared to a standard curve of known phosphate concentrations to express activity in FTUs. The absorbance was measured in the supernatant (grey bars) and the cells (diagonally hatched bars) in different strains (M12548, T2633, T2634, T2635, T2636, T2637 and T2638). (FIG. 5B) FTU were compared between the different strains. The left vertical axis shows supernatant activity and the FTU for each strains is provided as the grey bars. The right axis shows cell-associated FTU activity and is provided as □ for each strains (M12548, T2633, T2634, T2635, T2636, T2637 and T2638). The values for the parent strain and the Pst1 tether cell associated activity were outside the range of the standard curve and therefore below the detection limit.

FIG. 6 provides the phytase activity in culture supernatant (grey bars) or associated with cells (diagonally hatched bars) for strains expressing Escherichia coli phytase fused with either an N- or C-terminal tether. Supernatant was incubated with 5 mM sodium phytate solution pH 5.5 for 30 minutes and cells were incubated in the same solution for 2 hours. Results are shown as the optical density at 700 nm in function of each strain (M11312, T2705 and T2706).

FIG. 7 provides the phytase activity in culture supernatant (grey bars) or associated with cells (diagonally hatched bars) for strains expressing E. coli phytase fused with either an N-terminal tether with or without overexpression of AGA1, and compared to E. coli phytase fused with a C-terminal Sed1 tether. Supernatant was incubated with 5 mM sodium phytate solution pH 5.5 for 30 minutes and cells were incubated in the same solution for 2 hours. Results are shown as the optical density at 700 nm in function of each strain (M12550, M12795, M12983 and T2816).

FIG. 8 provides the wheat starch activity of strains expressing maltogenic amylase. Results are provided as the ratio of absorbance at 450 nm/optical density at 600 nm for the whole culture (left bars), the supernatant (middle bars) and washed cells (right bars) for the different strains (M10474, M13819, M13822, M14851, T4328, T4329, T4330, M12962, T4336, T4337 and T4338). Data for “M” strains are the average of duplicate cultures. Data for “T” strains include the average activity across cultures of seven transformations isolates. Expression type 1 refers to the presence of an invertase signal peptide and a Spi1 tether to generate a tethered enzyme. Expression type 2 refers to the presence of an invertase signal peptide and the absence of a tether to generate a secreted enzyme. Expression type 3 refers to the absence of a signal peptide and the absence of a tether to generate an intracellular enzyme.

FIG. 9 shows an SDS-PAGE gel of total protein samples of the commercial enzyme Novamyl® and supernatants of several samples of the strain M15532: yeast cream (˜20% solids), autolyzed cream after incubation at 55° C. for 48 hours and cream homogenized by bead-milling. The arrow points to a major band of the same molecular weight as the enzyme Novamyl® present in all M15532 samples, especially after autolysis or bead-milling to release intracellular enzyme.

FIG. 10 shows an SDS-PAGE of the enzyme Novamyl® and maltogenic amylase purified from two strains that express enzyme without signal peptide (predicted intracellular enzyme): M14851 and M15532. Results were generated in non-reducing (columns 3 to 5) and reducing (columns 7 to 9) conditions.

FIG. 11 provides an embodiment of the processes of the present disclosure for making different yeast products.

FIG. 12 shows the alpha-amylase activity associated with the cells of yeast strains expressing various chimeric proteins comprising a thermo-tolerant alpha-amylase derived from Pyrococcus furiosus (SEQ ID NO: 71) in combination with different tethering moieties derived from the SPI1 protein or associated truncations (M15774, M15771, M15777, M15772 and M15222) compared to a control strain (M2390). Results are shown as the absorbance at 540 nm in function of the yeast strain used.

FIG. 13 shows the alpha-amylase activity associated with cells of yeast strains expressing various chimeric proteins comprising an alpha-amylases derived from Thermococcus hydrothermalis (SEQ ID NO: 72) in combination with different tethering moieties derived from the CCW12 protein or associated truncations (M15773, M15776, M16251 and M15215) compared to a control strain (M2390). Results are shown as the absorbance at 540 nm in function of the yeast strain used.

FIG. 14 shows the alpha-amylase activity associated with the cells of yeast strains expressing various chimeric proteins comprising an alpha-amylase derived from T. hydrothermalis (SEQ ID NO: 72) in combination with a tethering moiety derived from the CCW12 protein and different linkers (M15785, M15786, M15782, M16252, M16221 and M16222) compared to a control strain (M2390). Results are shown as the absorbance at 540 nm in function of the yeast strain.

FIG. 15 shows the alpha-amylase activity associated with the cells of yeast strains expressing various chimeric proteins comprising an alpha-amylase derived from P. furiosus (SEQ ID NO: 71), a tethering moiety derived from the SPI1 protein and different linkers (M15784, M15778, M15779, M15787, M15780, M15788 and M15783) compared to a control strain (M2390). Results are shown as the absorbance at 540 nm in function of the yeast strain.

FIG. 16 shows the glucose oxidase (GO) activity associated with the whole culture (grey bars), washed cells (diagonal hatch bars) or the supernatant of disrupted washed cells (white bars) of yeast strains expressing a glucose oxidase derived from Aspergillus niger, expressed in a secreted form (M16780) or intracellularly (M16273) compared to a negative control strain (M10474) and a positive control amount of a commercially available purified glucose oxidase (positive control, Gluzyme Mono®). Results are shown as absorbance at 510 nm in function of the yeast strain/control used.

FIG. 17 shows the glucose oxidase (GO) activity associated with the whole culture (grey bars), washed cells (diagonal hatch bars) of yeast strains expressing a glucose oxidase derived from Aspergillus niger, expressed in a secreted form (M16780) or intracellularly (M16273). Results are shown as absorbance at 510 nm (corrected to remove the absorbance associated with control strain M10474) in function of the yeast strain used.

FIG. 18 shows the fungal amylase (FA) activity associated with the whole culture (grey bars), washed cells (diagonal bars) or the supernatant of disrupted washed cells (white bars) of yeast strains expressing a fungal amylase derived from Aspergillus oryzae expressed in a secreted form with a different signal peptides (S. cerevisiae invertase for M16772, A. oryzae native alpha-amylase signal peptide for M16540) compared to a negative control strain (M10474) and a positive control amount of a commercially available purified fungal alpha-amylase (positive control, Fungamyl®). Results are shown as absorbance at 540 nm in function of the yeast strain/control used.

FIG. 19 shows the fungal amylase (FA) activity associated with the whole culture (grey bars), washed cells (diagonal hatch bars) or the supernatant of disrupted washed cells (white bars) of yeast strains expressing a fungal amylase derived from Aspergillus oryzae expressed in a secreted form with a different signal peptides (S. cerevisiae invertase for M16772, A. oryzae native alpha-amylase signal peptide for M16540). Results are shown as absorbance at 540 nm (corrected to remove the absorbance associated with control strain M10474) in function of the yeast strain used.

### DETAILED DESCRIPTION

The present disclosure provides recombinant yeast host cells expressing a cell-associated heterologous protein during its propagation phase. As used in the context of the present disclosure, the expression “propagation phase” refers to an expansion phase of a commercial process in which the yeasts are propagated under aerobic conditions to maximize the conversion of a substrate into biomass. In some instances, the propagated biomass can be used in a following fermenting step (usually under anaerobic conditions) to maximize the production produce one or more desired metabolite. Advantageously, because the recombinant yeast host cell of the present disclosure expresses a cell-associated heterologous protein, it provides a yeast composition or a yeast product enriched in the heterologous protein, when compared to a recombinant yeast host cell expressing the heterologous protein in a free form (which is not cell-associated or non-tethered). In an embodiment, the yeast composition or the yeast product can comprise at least 1% (in dry weight) of the heterologous protein of the total proteins of the yeast composition or of the yeast product. In some embodiments, the yeast composition or the yeast product comprises at least 0.1 weight % of the heterologous protein when compared to the total weight of the proteins of the recombinant yeast host cell, the yeast composition or the yeast product. In some embodiments, the yeast composition or the yeast product comprises at least 0.001 g of the heterologous protein when compared to the total weight of the proteins of the recombinant yeast host cell, the yeast composition or the yeast product. In some embodiments, the yeast composition or the yeast product comprises at least 0.05 weight % of the heterologous protein when compared to the total weight of the recombinant yeast host cell. In some embodiments, the yeast composition or the yeast product comprises at least 0.0005 g of the heterologous protein/g of the dry weight of the recombinant yeast host cell. In some embodiments in which the heterologous protein is an enzyme, the yeast composition or the yeast product provides a minimal enzymatic activity of at least 50 enzymatic activity units/g of dry cell weight of the recombinant yeast host cell or/g of total proteins of the recombinant yeast host cell. In an embodiment, the cell-associated activity of the cell-associated heterologous protein is at least a 1, 2, 3, 4, 5, 6, 7, 8, 9, 10 or even 25 fold increase when compared with the cell-associated activity of a corresponding heterologous protein expressed in a free form (e.g., secreted). In some embodiments, the recombinant yeast host cell has at least 5, 10, 15, 20, 25, 20, 35, 40, 45, 50% of more of the heterologous protein when compared to a yeast host cell which does not express the heterologous protein (but which nevertheless expresses a corresponding native protein). In an embodiment, for a cell-associated heterologous protein, the ratio of the activity associated with the cells compared to the activity associated with the cell culture supernatant (e.g., free) is higher than 1:33, and it is, for example, between about 1:12 to 1:1.4. In another embodiment, for a cell-associated heterologous protein, the percentage of the activity associated with the cells (when compared to the total activity) is any percentage between 8 and 42%. The recombinant yeast host cells of the present disclosure are advantageous because they provide a lower cost source of enzyme activity than the purified products that are traditionally used. Furthermore, the activity of the heterologous protein in the recombinant yeast host cells can advantageously be easily measured, dosed and formulated prior to their inclusion in an industrial process.

Recombinant Yeast Host Cells

The recombinant yeast host cells of the present disclosure are intended to be used in the processes for making a yeast composition that can be used in various processes for making yeast products. The recombinant yeast host cell of the present disclosure (and, by the same token the yeast composition and the yeast product) comprises the heterologous protein in a cell-associated form, either in an intracellular form or associated with its membrane. As used in the context of the present disclosure, a “yeast composition” is a composition comprising the recombinant yeast host cell of the present disclosure which has been propagated. The yeast combination can be used, for example, in a following fermentation (to provide the heterologous protein in situ during fermentation) or to make a yeast product. In an embodiment, the recombinant yeast host cell is provided in an active or in a semi-active form in the yeast composition. For example, an embodiment of the yeast composition is a cream made from the recombinant yeast host cell of the present disclosure.

As also used in the context of the present disclosure, a “yeast product” is a composition comprising a product made by the recombinant yeast host cell of the present disclosure and comprising the heterologous protein. In an embodiment, a yeast product can be provided as an inactive form in the yeast provide cell of the present disclosure. In yet another embodiment, the yeast product can be a metabolite produced by the recombinant yeast host cell of the present disclosure, for example, an heterologous protein produced by the recombinant yeast host cell.

The recombinant yeast host cells of the present disclosure can optionally be used in a fermentation process. In an embodiment, the fermentation process can be a relatively long one and the recombinant yeast host cells can be used, for example, in making biofuels, distilling products, wine and beer. In another embodiment, the fermentation process can be a relatively short one and the recombinant yeast host cells can be used, for example, in making yeast-leavened bakery products.

The recombinant yeast host cells of the present disclosure can also be used in a process which does not include a fermentation step. For example, the recombinant yeast host cell can be used for making food and beverages (e.g., non-yeast-leavened (chemically-leavened) bakery products, dairy products, yeast extracts, juices, fat and oils as well as starch), feed or other industrial products (e.g., detergents, textiles, leather, pulp and paper, oil and gas and/or biopolymers).

The recombinant yeast host cells of the present disclosure can be provided in an active form (e.g., liquid, compressed, or fluid-bed dried yeast), in a semi-active form (e.g., liquid, compressed, or fluid-bed dried), in an inactive form (e.g., drum- or spray-dried) as well as a mixture therefore. For example, the recombinant yeast host cells can be a combination of active and semi-active or inactive forms to provide the ratio and dose of the heterologous protein required for making the yeast composition.

The present disclosure concerns recombinant yeast host cells that have been genetically engineered. The genetic modification(s) is(are) aimed at increasing the expression of a specific targeted gene (which is considered heterologous to the yeast host cell) and can be made in one or multiple (e.g., 1, 2, 3, 4, 5, 6, 7, 8 or more) genetic locations. In the context of the present disclosure, when recombinant yeast cell is qualified as being “genetically engineered”, it is understood to mean that it has been manipulated to add at least one or more heterologous or exogenous nucleic acid residue. In some embodiments, the one or more nucleic acid residues that are added can be derived from an heterologous cell or the recombinant host cell itself. In the latter scenario, the nucleic acid residue(s) is (are) added at one or more genomic location which is different than the native genomic location. The genetic manipulations did not occur in nature and are the results of in vitro manipulations of the yeast.

When expressed in recombinant yeast host cells, the heterologous proteins described herein are encoded on one or more heterologous nucleic acid molecules. The term “heterologous” when used in reference to a nucleic acid molecule (such as a promoter, a terminator or a coding sequence) or a protein refers to a nucleic acid molecule or a protein that is not natively found in the recombinant host cell. “Heterologous” also includes a native coding region/promoter/terminator, or portion thereof, that was removed from the source organism and subsequently reintroduced into the source organism in a form that is different from the corresponding native gene, e.g., not in its natural location in the organism's genome. The heterologous nucleic acid molecule is purposively introduced into the recombinant host cell. For example, a heterologous element could be derived from a different strain of host cell, or from an organism of a different taxonomic group (e.g., different kingdom, phylum, class, order, family genus, or species, or any subgroup within one of these classifications).

The heterologous nucleic acid molecule present in the recombinant host cell can be integrated in the host cell's genome. The term “integrated” as used herein refers to genetic elements that are placed, through molecular biology techniques, into the genome of a host cell. For example, genetic elements can be placed into the chromosomes of the host cell as opposed to in a vector such as a plasmid carried by the host cell. Methods for integrating genetic elements into the genome of a host cell are well known in the art and include homologous recombination. The heterologous nucleic acid molecule can be present in one or more copies (e.g., 2, 3, 4, 5, 6, 7, 8 or even more copies) in the yeast host cell's genome. Alternatively, the heterologous nucleic acid molecule can be independently replicating from the yeast's genome. In such embodiment, the nucleic acid molecule can be stable and self-replicating.

Suitable yeast host cells that can be used in the context of the present disclosure can be, for example, from the genus Saccharomyces, Kluyveromyces, Arxula, Debaryomyces, Candida, Pichia, Phaffia, Schizosaccharomyces, Hansenula, Kloeckera, Schwanniomyces, Torula or Yarrowia. Suitable yeast species can include, for example, S. cerevisiae, S. bulderi, S. barnetti, S. exiguus, S. uvarum, S. diastaticus, C. utilis, K. lactis, K. marxianus or K. fragilis. In some embodiments, the yeast is selected from the group consisting of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In one particular embodiment, the yeast is Saccharomyces cerevisiae. In some embodiments, the host cell can be an oleaginous yeast cell. For example, the oleaginous yeast host cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiment, the host cell can be an oleaginous microalgae host cell (e.g., for example, from the genus Thraustochytrium or Schizochytrium). In an embodiment, the recombinant yeast host cell is from the genus Saccharomyces and, in some embodiments, from the species Saccharomyces cerevisiae.

The recombinant yeast host cells of the present disclosure include an heterologous nucleic acid molecule intended to allow the expression (e.g., encoding) of one or more heterologous proteins. In an embodiment, the heterologous protein is an heterologous enzyme. In the context of the present application, the heterologous enzyme can be, without limitation, an heterologous oxidoreductase, an heterologous transferase, an heterologous hydrolase, an heterologous lyase, an heterologous isomerase, an heterologous phosphatase and/or an heterologous ligase.

As used in the context of the present disclosure, the expression “oxidoreductase” (also referred to as an oxidase, E.C. 1) refers to a protein having enzymatic activity and capable of catalyzing the transfer of electrons from one molecule (the reductant or the electron donor) to another (the oxidant or the electron acceptor). In an embodiment, the oxidoreductase is a hexose oxidase (E.C. 1.1.3.5), for example, the hexose oxidase can be a glucose oxidase (E.C. 1.1.3.4). In some embodiments, oxidases (such as glucose oxidases) can improve dough machinability. In an embodiment, the one or more oxidoreductases can be a glucose oxidase from Aspergillus niger (and have, for example, the amino acid sequence of SEQ ID NO: 44 or 103, a variant thereof or a fragment thereof). Oxidoreductases can be used in fermentation processes for making biofuels, distilling products, wine, beer and yeast-leavened bakery products. Oxidoreductases can be used for making food and beverages (e.g., non-yeast-leavened (chemically-leavened) bakery products), feed or other industrial products (e.g., detergents, textiles, leather, pulp and paper, oil and gas and/or biopolymers).

As used in the context of the present disclosure, the expression “transferase” (E.C. 2) refers to a protein having enzymatic activity and capable of catalyzing the transfer of specific functional groups (e.g., a methyl or glycosyl group for example) from one molecule (called the donor) to another (called the acceptor). For example, the transferases can be acyltransferases (E.C. 2.3 such as transglutaminases (E.C. 2.3.2.13) for example) or glycosyltransferases (E.C. 2.4 such as amylomaltases (E.C. 2.4.1.3) for example). A transglutaminase can be used in baking goods to improve dough strength.

As used in the context of the present disclosure, the expression “lyase” (E.C. 4) refers to a protein having enzymatic activity and capable of catalyzing the elimination of various chemical bonds by means other than hydrolysis (e.g., a “substitution” reaction) and oxidation. For example, the lyase can be a malolactic enzyme (EC 4.1.1.101), Acetolactate decarboxylase (or, alpha-acetolactate decarboxylase, EC 4.1.1.5) and/or a pectate lyase (E.C. 4.2.2.2). Lyases can be used in fermentation processes for making biofuels, distilling products, wine, beer and yeast-leavened bakery products. Lyases can also be used for making food and beverages (e.g., non-yeast-leavened (chemically-leavened) bakery products), feed or other industrial products (e.g., detergents, textiles, leather, pulp and paper, oil and gas and/or biopolymers).

As used in the context of the present disclosure, the expression “isomerase” (E.C. 5) refers to a protein having enzymatic activity and capable of catalyzing the conversion a molecule from one isomer to another. For example, the isomerase can be a glucose isomerase (E.C. 5.1.3) or xylose isomerase (EC 5.1.3.5). Isomerases can be used in fermentation processes for making biofuels, distilling products, wine, beer and yeast-leavened bakery products. Isomerases can also be used for making food and beverages (e.g., non-yeast-leavened (chemically-leavened) bakery products), feed or other industrial products (e.g., detergents, textiles, leather, pulp and paper, oil and gas and/or biopolymers).

As used in the context of the present disclosure, the expression “ligase” (E.C. 6) refers to a protein having enzymatic activity and capable of catalyzing the joining of two molecules by forming a new chemical bond. For example, the ligase can be an urea amidolyase (E.C. EC 6.3.4.6). Ligases can be used in fermentation processes for making biofuels, distilling products, wine, beer and yeast-leavened bakery products. Ligases can also be used for making food and beverages (e.g., non-yeast-leavened (chemically-leavened) bakery products), feed or other industrial products (e.g., detergents, textiles, leather, pulp and paper, oil and gas and/or biopolymers).

As used in the context of the present disclosure, the expression “hydrolase” (E.C. 3) refers to a protein having enzymatic activity and capable of catalyzing the hydrolysis of a chemical bound. For example, the hydrolase can be an esterase (E.C. 3.1 for example lipase, phospholipase A1 and/or phospholipase A2), can cleaved C—N non-peptide bonds (E.C. 3.5 for example an asparaginase), can be a glycosylase (E.C. 3.2 for example an amylase (E.C. 3.2.1.1), a glucanase, a glycosidase (E.C. 3.2.1), a cellulase (E.C. 3.2.1.4)), a pectinase and/or a lactase (E.C. 3.2.1.108)), a protease (E.C. 3.4 for example a bacterial protease, a plant protease or a fungal protease). When the hydrolase is an amylase, it can be, for example, a fungal alpha amylase, a bacterial alpha amylase, a maltogenic alpha amylase, a maltotetrahydrolase, a plant (e.g., barley) alpha or beta amylase and/or a glucoamylase. When the hydrolase is a glycosidase, it can be, for example, a beta glucosidase. When the hydrolase is a cellulase, it can be, for example, a cellulase, an hemicellulase and/or a xylanase.

As used herein, the expression “phosphatase” refers to a protein having enzymatic activity and capable, in the presence of water, of catalyzing the cleavage of a phosphoric acid monoester into a phosphate ion and an alcohol. An embodiment of a phosphatase is a phytase, a protein having enzymatic activity and capable of catalyzing the hydrolysis of phytic acid (myo-inositol hexakisphosphate) into inorganic phosphorus. There are four distinct classes of phytase: histidine acid phosphatases (HAPS), β-propeller phytases, purple acid phosphatases and protein tyrosine phosphatase-like phytases (PTP-like phytases). Phytic acid has six phosphate groups that may be released by phytases at different rates and in different order. Phytases hydrolyze phosphates from phytic acid in a stepwise manner, yielding products that again become substrates for further hydrolysis. Phytases have been grouped based on the first phosphate position of phytic acid that is hydrolyzed: are 3-phytase (EC 3.1.3.8), 4-phytase (EC 3.1.3.26) and 5-phytase (EC 3.1.3.72). In an embodiment, the phytase is derived from a bacterial species, such as, for example, a Citrobacter sp. or an Escherichia sp. In a specific embodiment, the heterologous phytase is derived from a Citrobacter sp., such as for example Citrobacter braakii and can have, for example, the amino acid sequence of SEQ ID NO: 66, a variant thereof or a fragment thereof. In another embodiment, the heterologous phytase is derived from an Escherichia sp., such as, for example, Escherichia coli and can have, for example, the amino acid sequence of SEQ ID NO: 67, a variant thereof or a fragment thereof.

As used herein, the expression “amylolytic enzyme” refers to a class of enzymes capable of hydrolyzing starch or hydrolyzed starch. Amylolytic enzymes include, but are not limited to alpha-amylases (EC 3.2.1.1, sometimes referred to fungal alpha-amylase, see below), maltogenic amylase (EC 3.2.1.133), glucoamylase (EC 3.2.1.3), glucan 1,4-alpha-maltotetraohydrolase (EC 3.2.1.60), pullulanase (EC 3.2.1.41), iso-amylase (EC 3.2.1.68) and amylomaltase (EC 2.4.1.25). In an embodiment, the one or more amylolytic enzymes can be an alpha-amylase from Aspergillus oryzae (and have, for example, the amino acid sequence of SEQ ID NO: 2 or 105, a variant thereof or a fragment thereof), a maltogenic alpha-amylase from Geobacillus stearothermophilus (and have, for example, the amino acid sequence of SEQ ID NO: 1, 51, 65, or 108, a variant thereof or a fragment thereof), a glucoamylase from Saccharomycopsis fibuligera (and have, for example, the amino acid sequence of SEQ ID NO: 3, a variant thereof or a fragment thereof), a glucan 1,4-alpha-maltotetraohydrolase from Pseudomonas saccharophila (and have, for example, the amino acid sequence of SEQ ID NO: 4, a variant thereof or a fragment thereof), a pullulanase from Bacillus naganoensis (and have, for example, the amino acid sequence of SEQ ID NO: 5, a variant thereof or a fragment thereof), a pullulanase from Bacillus acidopullulyticus (and have, for example, the amino acid sequence of SEQ ID NO: 6, a variant thereof or a fragment thereof), an iso-amylase from Pseudomonas amyloderamosa (and have, for example, the amino acid sequence of SEQ ID NO: 7, a variant thereof or a fragment thereof), and/or amylomaltase from Thermus thermophilus (and have, for example, the amino acid sequence of SEQ ID NO: 8, a variant thereof or a fragment thereof).

As used herein, the expression “cellulase/hemi-cellulase” refers to a class of enzymes capable of hydrolyzing, respectively, cellulose or hemi-cellulose. Cellulases/hemi-cellulases include, but are not limited to a cellulase (E.C. 3.2.1.4) and an endoB(1,4)D-xylanase (E.C. 3.2.1.8). In an embodiment, the one or more cellulase/hemi-cellulase can be a cellulase from Penicillium funiculosum (and have, for example, the amino acid sequence of SEQ ID NO: 42, a variant thereof or a fragment thereof) and/or an endoB(1,4)D-xylanase from Rasamsonia emersonii (and have, for example, the amino acid sequence of SEQ ID NO: 43, a variant thereof or a fragment thereof).

As used herein, the expression “lipase” refers to a class of enzymes capable of hydrolyzing lipids. In an embodiment, the one or more lipase can be a triacylglycerol lipase from Thermomyces lanuginosis (and have, for example, the amino acid sequence of SEQ ID NO: 45, a variant thereof or a fragment thereof), a phospholipase A2 from Sus scrofa (and have, for example, the amino acid sequence of SEQ ID NO: 46, a variant thereof or a fragment thereof), a phospholipase A2 from Streptomyces vialaceoruber (and have, for example, the amino acid sequence of SEQ ID NO: 47, a variant thereof or a fragment thereof) and/or a phospholipase A2 from Aspergillus oryzea (and have, for example, the amino acid sequence of SEQ ID NO: 48, a variant thereof or a fragment thereof).

As used in the present disclosure, the term “maltogenic amylase” refers to a polypeptide capable of hydrolyzing starch or hydrolyzed starch into maltose. Maltogenic amylases include, but are not limited to fungal alpha-amylases (derived, for example, from Aspergillus sp. (e.g., A. Niger, A. kawachi, and A. oryzae); Trichoderma sp. (e.g., T. reesie), Rhisopus sp., Mucor sp., and Penicillium sp.), acid stable fungal amylase (derive, for example, from Aspergillus niger), β-amylases (derived, for example, from plant (wheat, barley, rye, shorgum, soy, sweet potato, rice) and microorganisms (Bacillus cereus, Bacillus polymixa, Bacillus megaterium, Arabidopsis thaliana), maltogenic amylases (E.C. 3.2.1.133) (derived, for example, from microorganisms such as Bacillus subtilis, Geobacillus stearothermophilus, Bacillus thermoalkalophilus, Lactobacillus gasseri, Thermus sp.). In a specific embodiment, the recombinant yeast host cells of the present disclosure include an heterologous nucleic acid molecule coding for the heterologous maltogenic amylase derived from Geobacillus stearothermophilus and having, for example, the amino acid sequence of SEQ ID NO: 1, 51, 65 or 108, a variant thereof or a fragment thereof.

The heterologous protein can be a variant of a known/native protein. A variant comprises at least one amino acid difference when compared to the amino acid sequence of the native/know protein. As used herein, a variant refers to alterations in the amino acid sequence that do not adversely affect the biological functions of the heterologous protein. A substitution, insertion or deletion is said to adversely affect the protein when the altered sequence prevents or disrupts a biological function associated with the heterologous protein. For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the heterologous protein. The protein variants have at least 50%, 55%, 60%, 65%, 70%, 75%, 80%, 85%, 90%, 95%, 96%, 97%, 98% or 99% identity to the heterologous protein described herein. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant heterologous protein described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. A “variant” of the heterologous protein can be a conservative variant or an allelic variant.

The heterologous protein can be a fragment of a known/native protein or fragment of a variant of a known/native protein. In an embodiment, the fragment corresponds to the known/native protein to which the signal peptide sequence has been removed. In some embodiments, heterologous protein “fragments” have at least at least 100, 200, 300, 400, 500, 600, 700, 800, 900 or more consecutive amino acids of the heterologous protein. A fragment comprises at least one less amino acid residue when compared to the amino acid sequence of the known/native heterologous protein and still possess the enzymatic activity of the full-length heterologous protein. In an embodiment, the fragment corresponds to the amino acid sequence of the protein lacking the signal peptide. In some embodiments, fragments of the heterologous protein can be employed for producing the corresponding full-length heterologous by peptide synthesis. Therefore, the fragments can be employed as intermediates for producing the full-length proteins.

In the recombinant yeast host cell of the present disclosure, the heterologous protein is “cell-associated” to the recombinant yeast host cell because it is designed to be expressed and remain physically associated with the recombinant yeast host cells. In an embodiment, the heterologous protein can be expressed inside the recombinant yeast host cell (intracellularly). In such embodiment, the heterologous protein does not need to be associated to the recombinant yeast host cell's wall. When the heterologous protein is intended to be expressed intracellularly, its signal sequence, if present in the native sequence, can be deleted to allow intracellular expression.

In another embodiment, the heterologous protein of the present disclosure can be secreted, but when it is, it must remain physically associated with the recombinant yeast host cell. In an embodiment, at least one portion (usually at least one terminus) of the heterologous protein is bound, covalently, non-covalently and/or electrostatically for example, to cell wall (and in some embodiments to the cytoplasmic membrane). For example, the heterologous protein can be modified to bear one or more transmembrane domains, to have one or more lipid modifications (myristoylation, palmitoylation, farnesylation and/or prenylation), to interact with one or more membrane-associated protein and/or to interactions with the cellular lipid rafts. While the heterologous protein may not be directly bound to the cell membrane or cell wall (e.g., such as when binding occurs via a tethering moiety), the protein is nonetheless considered a “cell-associated” heterologous protein according to the present disclosure.

In some embodiments, the heterologous protein can be expressed to be located at and associated to the cell wall of the recombinant yeast host cell. In some embodiments, the heterologous protein is expressed to be located at and associated to the external surface of the cell wall of the host cell. Recombinant yeast host cells all have a cell wall (which includes a cytoplasmic membrane) defining the intracellular (e.g., internally-facing the nucleus) and extracellular (e.g., externally-facing) environments. The heterologous protein can be located at (and in some embodiments, physically associated to) the external face of the recombinant yeast host's cell wall and, in further embodiments, to the external face of the recombinant yeast host's cytoplasmic membrane. In the context of the present disclosure, the expression “associated to the external face of the cell wall/cytoplasmic membrane of the recombinant yeast host cell” refers to the ability of the heterologous protein to physically integrate (in a covalent or non-covalent fashion), at least in part, in the cell wall (and in some embodiments in the cytoplasmic membrane) of the recombinant yeast host cell. The physical integration can be attributed to the presence of, for example, a transmembrane domain on the heterologous protein, a domain capable of interacting with a cytoplasmic membrane protein on the heterologous protein, a post-translational modification made to the heterologous protein (e.g., lipidation), etc.

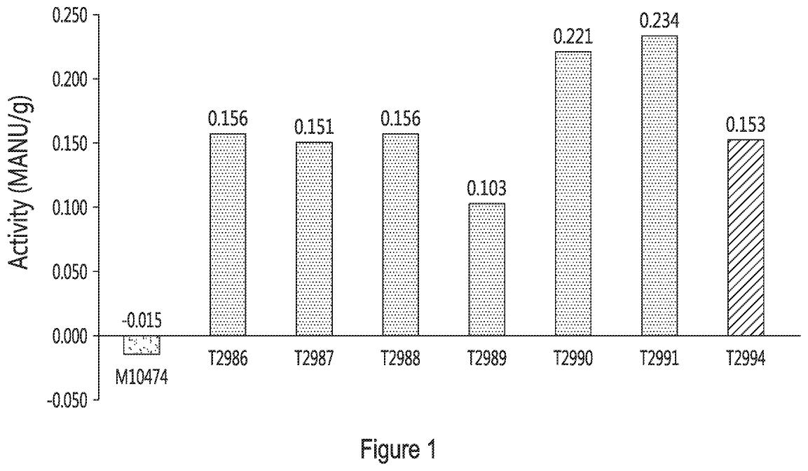

Some heterologous proteins have the intrinsic ability to locate at and associate to the cell wall of a recombinant yeast host cell (e.g., being cell-associated). One example of an heterologous protein having the intrinsic ability of being cell-associated is shown in FIG. 1A moiety (e.g., strain T2994 in FIG. 1). In this figure, results are presented for the maltogenic alpha-amylase of Geobacillus stearothermophilus expressed in S. cerevisiae in the absence of a tethering moiety and clearly show that this heterologous protein is intrinsically “cell-associated” and exhibits enzymatic activity (e.g., maltogenic alpha-amylase activity).

However, in some circumstances, it may be warranted to increase or provide cell association to some heterologous proteins because they exhibit insufficient intrinsic cell association or simply lack intrinsic cell association. In such embodiment, it is possible to provide the heterologous protein as a chimeric construct by combining it with a tethering amino acid moiety which will provide or increase attachment to the cell wall of the recombinant yeast host cell. In such embodiment, the chimeric heterologous protein will be considered “tethered”. It is preferred that the amino acid tethering moiety of the chimeric protein be neutral with respect to the biological activity of the heterologous protein, e.g., does not interfere with the biological activity (such as, for example, the enzymatic activity) of the heterologous protein. In some embodiments, the association of the amino acid tethering moiety with the heterologous protein can increase the biological activity of the heterologous protein (when compared to the non-tethered, “free” form).

In an embodiment, a tethering moiety can be used to be expressed with the heterologous protein to locate the heterologous protein to the wall of the recombinant yeast host cell. Various tethering amino acid moieties are known art and can be used in the chimeric proteins of the present disclosure. The tethering moiety can be a transmembrane domain found on another protein and allow the chimeric protein to have a transmembrane domain. In such embodiment, the tethering moiety can be derived from the FLO1 protein (having, for example, the amino acid sequence of SEQ ID NO: 10, a variant thereof or a fragment thereof or being encoded by the nucleic acid sequence of SEQ ID NO: 9).

In still another example, the amino acid tethering moiety can be modified post-translation to include a glycosylphosphatidylinositol (GPI) anchor and allow the chimeric protein to have a GPI anchor. GPI anchors are glycolipids attached to the terminus of a protein (and in some embodiments, to the carboxyl terminus of a protein) which allows the anchoring of the protein to the cytoplasmic membrane of the cell membrane. Tethering amino acid moieties capable of providing a GPI anchor include, but are not limited to those associated with/derived from a SED1 protein (having, for example, the amino acid sequence of SEQ ID NO: 12, a variant thereof or a fragment thereof or being encoded by the nucleic acid sequence of SEQ ID NO: 11), a TIR1 protein (having, for example, the amino acid sequence of SEQ ID NO: 14, a variant thereof or a fragment thereof or being encoded by the nucleic acid sequence of SEQ ID NO: 13), a CWP2 protein (having, for example, the amino acid sequence of SEQ ID NO: 16, a variant thereof or a fragment thereof or being encoded by the nucleic acid sequence of SEQ ID NO: 15), a CCW12 protein (having, for example, the amino acid sequence of SEQ ID NO: 18 or 84, a variant thereof or a fragment thereof or being encoded by the nucleic acid sequence of SEQ ID NO: 17), a SPI1 protein (having, for example, the amino acid sequence of SEQ ID NO: 20 or 74, a variant thereof or a fragment thereof or being encoded by the nucleic acid sequence of SEQ ID NO: 19), a PST1 protein (having, for example, the amino acid sequence of SEQ ID NO: 22, a variant thereof or a fragment thereof or being encoded by the nucleic acid sequence of SEQ ID NO: 21) or a combination of a AGA1 and a AGA2 protein (having, for example, the amino acid sequence of SEQ ID NO: 24, a variant thereof or a fragment thereof or being encoded by the nucleic acid sequence of SEQ ID NO: 23 or having, for example, the amino acid sequence of SEQ ID NO: 26, a variant thereof or a fragment thereof or being encoded by the nucleic acid sequence of SEQ ID NO: 25). In an embodiment, the tethering moiety provides a GPI anchor and, in still a further embodiment, the tethering moiety is derived from the SPI1 protein (having, for example, the amino acid sequence of SEQ ID NO: 20, a variant thereof or a fragment thereof or being encoded by the nucleic acid sequence of SEQ ID NO: 19) or the CCW12 protein (having, for example, the amino acid sequence of SEQ ID NO: 18, a variant thereof or a fragment thereof or being encoded by the nucleic acid sequence of SEQ ID NO: 17).

In an embodiment, the tethering moiety is a fragment of the SPI1 protein that retained its ability to localize to the cell's membrane. The fragment of the SPI1 protein comprises less than 129 amino acid consecutive residues of the amino acid sequence of SEQ ID NO: 74. For example, the tethering moiety fragment from the SPI1 protein can comprise at least 10, 20, 21, 30, 40, 50, 51, 60, 70, 80, 81, 90, 100, 110, 111 or 120 consecutive amino acid residues from the amino acid sequence of SEQ ID NO: 74. In yet another embodiment, the tethering moiety fragment from the SPI1 protein can comprise or consist essentially of the amino acid sequence set forth in any one of SEQ ID NOs: 76, 78, 80 or 82.

In another embodiment, the tethering moiety is a fragment of a CCW12 protein that retained its ability to localize to the cell's membrane. The fragment of the CCW12 protein comprises less than 112 amino acid consecutive residues of the amino acid sequence of SEQ ID NO: 84. For example, the tethering moiety fragment from the CCW12 protein can comprise at least 10, 20, 24, 30, 40, 49, 50, 60, 70, 74, 80, 90, 99, 100 or 110 consecutive amino acid residues from the amino acid sequence of SEQ ID NO: 84. In yet another embodiment, the tethering moiety fragment from the CCW12 protein can comprise or consist essentially of the amino acid sequence set forth in any one of SEQ ID NOs: 86, 88, 90 or 92.

The tethering amino acid moiety can be a variant of a known/native tethering amino acid moiety, for example a variant of the tethering amino acid moiety having the amino acid sequence of SEQ ID NOs: 10, 12, 14, 16, 18, 20, 22, 24, 26, 74, 76, 78, 80, 82, 84, 86, 88, 90 or 92. A variant comprises at least one amino acid difference when compared to the amino acid sequence of the native tethering amino acid moiety. As used herein, a variant refers to alterations in the amino acid sequence that do not adversely affect the biological functions of the tethering amino acid moiety (e.g., the location on the external face and the anchorage of the heterologous protein in the cytoplasmic membrane). A substitution, insertion or deletion is said to adversely affect the protein when the altered sequence prevents or disrupts a biological function associated with the tethering amino acid moiety (e.g., the location on the external face and the anchorage of the heterologous protein in the cytoplasmic membrane). For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the tethering amino acid moiety. The tethering amino acid moiety variants have at least 50%, 55%, 60%, 65%, 70%, 75%, 80%, 85%, 90%, 95%, 96%, 97%, 98% or 99% identity to the tethering amino acid moieties described herein. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant tethering amino acid moieties described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. A “variant” of the tethering amino acid moiety can be a conservative variant or an allelic variant.

The tethering amino acid moiety can be a fragment of a known/native tethering amino acid moiety or fragment of a variant of a known/native tethering amino acid moiety (such as, for example, a fragment of the tethering amino acid moiety having the amino acid sequence of SEQ ID NO: 10, 12, 14, 16, 18, 20, 22, 24, 26, 74, 76, 78, 80, 82, 84, 86, 88, 90 or 92 or a variant thereof). Tethering amino acid moiety “fragments” have at least at least 10, 20, 30, 40, 50, 60, 70, 80, 90, 100 or more consecutive amino acids of the tethering amino acid moiety. A fragment comprises at least one less amino acid residue when compared to the amino acid sequence of the known/native tethering amino acid moiety and still possess the biological activity of the full-length tethering amino acid moiety (e.g., the location to the cell wall).

In embodiments in which an amino acid tethering moiety is desirable, the heterologous protein can be provided as a chimeric protein expressed by the recombinant yeast host cell and having one of the following formulae (provided from the amino (NH₂) to the carboxyl (COOH) orientation):

HP-L-TT (I) or

TT-L-HP (II)

In both of these formulae, the residue “HP” refers to the heterologous protein moiety, the residue “L” refers to the presence of an optional linker while the residue “TT” refers to an amino acid tethering moiety. In the chimeric proteins of formula (I), the amino terminus of the amino acid tether is located (directly or indirectly) at the carboxyl (COOH or C) terminus of the heterologous protein moiety. In the chimeric proteins of formula (II), the carboxy terminus of the amino acid tether is located (directly or indirectly) at the amino (NH₂ or N) terminus of the heterologous protein moiety.

When the amino acid linker (L) is absent, the tethering amino acid moiety is directly associated with the heterologous protein. In the chimeras of formula (I), this means that the carboxyl terminus of the heterologous protein moiety is directly associated (with an amide linkage) to the amino terminus of the tethering amino acid moiety. In the chimeras of formula (II), this means that the carboxyl terminus of the tethering amino acid moiety is directly associated (with an amide linkage) to the amino terminus of the heterologous protein.

In some embodiments, the presence of an amino acid linker (L) is desirable either to provide, for example, some flexibility between the heterologous protein moiety and the tethering amino acid moiety or to facilitate the construction of the heterologous nucleic acid molecule. As used in the present disclosure, the “amino acid linker” or “L” refer to a stretch of one or more amino acids separating the heterologous protein moiety HP and the amino acid tethering moiety TT (e.g., indirectly linking the heterologous protein HP to the amino acid tethering moiety TT). It is preferred that the amino acid linker be neutral, e.g., does not interfere with the biological activity of the heterologous protein nor with the biological activity of the amino acid tethering moiety. In some embodiments, the amino acid linker L can increase the biological activity of the heterologous protein moiety and/or of the amino acid tethering moiety. In instances in which the linker (L) is present in the chimeras of formula (I), its amino end is associated (with an amide linkage) to the carboxyl end of the heterologous protein moiety and its carboxyl end is associated (with an amide linkage) to the amino end of the amino acid tethering moiety. In instances in which the linker (L) is present in the chimeras of formula (II), its amino end is associated (with an amide linkage) to the carboxyl end of the amino acid tethering moiety and its carboxyl end is associated (with an amide linkage) to the amino end of the heterologous protein moiety. Various amino acid linkers exist and include, without limitations, (G)ₙ, (GS)ₙ; (GGS)ₙ; (GGGS)ₙ; (GGGGS)ₙ; (GGSG)ₙ; (GSAT)ₙ, wherein n=is an integer between 1 to 8 (or more). In an embodiment, the amino acid linker L is (GGGGS)ₙ (also referred to as G₄S) and, in still further embodiments, the amino acid linker L comprises more than one G₄S (SEQ ID NO: 41) motifs. For example, the amino acid linker L can be (G₄S)₃ and have the amino acid sequence of SEQ ID NO: 93. In another example, the amino acid linker L can be (G)₈ and have the amino acid sequence of SEQ ID NO: 94. In still another example, the amino acid linker L can be (G₄S)₈ and have the amino acid sequence of SEQ ID NO: 95.

The amino acid linker can also be, in some embodiments, GSAGSAAGSGEF (SEQ ID NO: 96).

Additional amino acid linkers exist and include, without limitations, (EAAK)ₙ and (EAAAK)ₙ, wherein n=is an integer between 1 to 8 (or more). In some embodiments, the one or more (EAAK)ₙ/(EAAAK)ₙ motifs can be separated by one or more additional amino acid residues. In an embodiment, the amino acid linker comprises one or more EA₂K (SEQ ID NO: 100) or EA₃K (SEQ ID NO: 101) motifs. In an embodiment, the amino acid linker can be (EAAK)₃ and has the amino acid sequence of SEQ ID NO: 97. In another embodiment, the amino acid linker can be (A(EAAAK)₄ALEA(EAAAK)₄A) and has the amino acid sequence of SEQ ID NO: 99.

Further amino acid linkers include those having one or more (AP)ₙ motifs wherein n=is an integer between 1 to 10 (or more). In an embodiment, the linker is (AP)₁₀ and has the amino acid of SEQ ID NO: 98.

In some embodiments, the linker also includes one or more HA tag (SEQ ID NO: 53).

Tools for Making the Recombinant Yeast Host Cell

In order to make the recombinant yeast host cells, heterologous nucleic acid molecules (also referred to as expression cassettes) are made in vitro and introduced into the yeast host cell in order to allow the recombinant expression of the heterologous protein.

The heterologous nucleic acid molecules of the present disclosure comprise a coding region for the heterologous polypeptide, e.g., the heterologous protein or a chimeric protein comprising same. A DNA or RNA “coding region” is a DNA or RNA molecule (preferably a DNA molecule) which is transcribed and/or translated into an heterologous protein in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The heterologous nucleic acid molecules described herein can comprise transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are control regions.

In some embodiments, the heterologous nucleic acid molecules of the present disclosure include a promoter as well as a coding sequence for an heterologous protein (including chimeric proteins comprising same). The heterologous nucleic acid sequence can also include a terminator. In the heterologous nucleic acid molecules of the present disclosure, the promoter and the terminator (when present) are operatively linked to the nucleic acid coding sequence of the heterologous protein (including chimeric proteins comprising same), e.g., they control the expression and the termination of expression of the nucleic acid sequence of the heterologous protein (including chimeric proteins comprising same). The heterologous nucleic acid molecules of the present disclosure can also include a nucleic acid coding for a signal peptide, e.g., a short peptide sequence for exporting the heterologous protein outside the host cell. When present, the nucleic acid sequence coding for the signal peptide is directly located upstream and is in frame with the nucleic acid sequence coding for the heterologous protein (including chimeric proteins comprising same).

In the heterologous nucleic acid molecule described herein, the promoter and the nucleic acid molecule coding for the heterologous protein (including chimeric proteins comprising same) are operatively linked to one another. In the context of the present disclosure, the expressions “operatively linked” or “operatively associated” refers to fact that the promoter is physically associated to the nucleotide acid molecule coding for the heterologous polypeptide in a manner that allows, under certain conditions, for expression of the heterologous protein from the nucleic acid molecule. In an embodiment, the promoter can be located upstream (5′) of the nucleic acid sequence coding for the heterologous protein. In still another embodiment, the promoter can be located downstream (3′) of the nucleic acid sequence coding for the heterologous protein. In the context of the present disclosure, one or more than one promoter can be included in the heterologous nucleic acid molecule. When more than one promoter is included in the heterologous nucleic acid molecule, each of the promoters is operatively linked to the nucleic acid sequence coding for the heterologous protein. The promoters can be located, in view of the nucleic acid molecule coding for the heterologous protein, upstream, downstream as well as both upstream and downstream.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein.

Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. Promoters which cause a gene to be expressed during the propagation phase of a yeast cell are herein referred to as “propagation promoters”. Propagation promoters include both constitutive and inducible promoters, such as, for example, glucose-regulated, molasses-regulated, stress-response promoters (including osmotic stress response promoters) and aerobic-regulated promoters. In the context of the present disclosure, it is important that the selected promoter allows the expression of the heterologous nucleic acid molecule during the propagation phase of the recombinant yeast host cell in order to allow a sufficient amount of cell-associated heterologous proteins to be expressed. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of the polymerase.

The promoter can be native or heterologous to the nucleic acid molecule encoding the heterologous polypeptide. The promoter can be heterologous or derived from a strain being from the same genus or species as the recombinant host cell. In an embodiment, the promoter is derived from the same genus or species of the yeast host cell and the heterologous polypeptide is derived from a different genus than the host cell. The promoter can be a single promoter or a combination of different promoters.

In the present disclosure, promoters allowing or favoring the expression of the heterologous proteins during the propagation phase of the recombinant yeast host cells are preferred. Yeasts that are facultative anaerobes, are capable of respiratory reproduction under aerobic conditions and fermentative reproduction under anaerobic conditions. In many commercial applications, yeast are propagated under aerobic conditions to maximize the conversion of a substrate to biomass. Optionally, the biomass can be used in a subsequent fermentation under anaerobic conditions to produce a desired metabolite. In the context of the present disclosure, it is important that the promoter or the combination of promoters present in the heterologous nucleic acid is/are capable of allowing the expression of the heterologous protein or its corresponding chimera during the propagation phase of the recombinant yeast host cell. This will allow the accumulation of the heterologous protein associated with the recombinant yeast host cell prior to any subsequent use in a fermentation (if any). In some embodiments, the promoter allows the expression of the heterologous protein or its corresponding chimera during the propagation phase, but not during the fermentation phase (if any) of the life cycle of the recombinant yeast host cell.

The promoters can be native or heterologous to the heterologous gene encoding the heterologous protein. The promoters that can be included in the heterologous nucleic acid molecule can be constitutive or inducible promoters (such as those described in Perez-Torrado et al., 2005). Inducible promoters include, but are not limited to glucose-regulated promoters (e.g., the promoter of the hxt7 gene (referred to as hxt7p) and having the nucleic acid sequence of SEQ ID NO: 30, a functional variant or a functional fragment thereof; the promoter of the ctt1 gene (referred to as ctt1p) and having the nucleic acid sequence of SEQ ID NO: 60, a functional variant or a functional fragment thereof; the promoter of the glo1 gene (referred to as glo1p) and having the nucleic acid sequence of SEQ ID NO: 59, a functional variant or a functional fragment thereof; the promoter of the ygp1 gene (referred to as ygp1p) and having the nucleic acid sequence of SEQ ID NO: 61, a functional variant or a functional fragment thereof; the promoter of the gsy2 gene (referred to as gsy2p) and having the nucleic acid sequence of SEQ ID NO: 53, a functional variant or a functional fragment thereof), molasses-regulated promoters (e.g., the promoter of the mol1 gene (referred to as mol1p) described in Praekelt et al., 1992 or having the nucleic acid sequence of SEQ ID NO: 64, a functional variant or a functional fragment thereof), heat shock-regulated promoters (e.g., the promoter of the glo1 gene (referred to as glo1p) and having the nucleic acid sequence of SEQ ID NO: 59, a functional variant or a functional fragment thereof; the promoter of the sti1 gene (referred to as sti1p) and having the nucleic acid sequence of SEQ ID NO: 56, a functional variant or a functional fragment thereof; the promoter of the ygp1 gene (referred to as ygp1p) and having the nucleic acid sequence of SEQ ID NO: 61, a functional variant or a functional fragment thereof; the promoter of the gsy2 gene (referred to as gsy2p) and having the nucleic acid sequence of SEQ ID NO: 53, a functional variant or a functional fragment thereof), oxidative stress response promoters (e.g., the promoter of the cup1 gene (referred to as cup1p) and having the nucleic acid sequence of SEQ ID NO: 58, a functional variant or a functional fragment thereof; the promoter of the ctt1 gene (referred to as ctt1p) and having the nucleic acid sequence of SEQ ID NO: 60, a functional variant or a functional fragment thereof; the promoter of the trx2 gene (referred to as trx2p) and having the nucleic acid sequence of SEQ ID NO: 55, a functional variant or a functional fragment thereof; the promoter of the gpd1 gene (referred to as gpd1p) and having the nucleic acid sequence of SEQ ID NO: 57, a functional variant or a functional fragment thereof; the promoter of the hsp12 gene (referred to as hsp12p) and having the nucleic acid sequence of SEQ ID NO: 63, a functional variant or a functional fragment thereof), osmotic stress response promoters (e.g., the promoter of the ctt1 gene (referred to as ctt1p) and having the nucleic acid sequence of SEQ ID NO: 60, a functional variant or a functional fragment thereof; the promoter of the glo1 gene (referred to as glo1p) and having the nucleic acid sequence of SEQ ID NO: 59, a functional variant or a functional fragment thereof; the promoter of the gpd1 gene (referred to as gpd1p) and having the nucleic acid sequence of SEQ ID NO: 57, a functional variant or a functional fragment thereof; the promoter of the ygp1 gene (referred to as ygp1p) and having the nucleic acid sequence of SEQ ID NO: 61, a functional variant or a functional fragment thereof) and nitrogen-regulated promoters (e.g., the promoter of the ygp1 gene (referred to as ygp1p) and having the nucleic acid sequence of SEQ ID NO: 61, a functional variant or a functional fragment thereof).

Promoters that can be included in the heterologous nucleic acid molecule of the present disclosure include, without limitation, the promoter of the tdh1 gene (referred to as tdh1p and having, for example, the nucleic acid sequence of SEQ ID NO: 27, a functional variant or a functional fragment thereof), of the hor7 gene (referred to as hor7p and having, for example, the nucleic acid sequence of SEQ ID NO: 28, a functional variant or a functional fragment thereof), of the hsp150 gene (referred to as hsp150p and having, for example, the nucleic acid sequence of SEQ ID NO: 29, a functional variant or a functional fragment thereof), of the hxt7 gene (referred to as hxt7p and having, for example, the nucleic acid sequence of SEQ ID NO: 30, a functional variant or a functional fragment thereof), of the gpm1 gene (referred to as gpm1p and having, for example, the nucleic acid sequence of SEQ ID NO: 31, a functional variant or a functional fragment thereof), of the pgk1 gene (referred to as pgk1p and having, for example, the nucleic acid sequence of SEQ ID NO: 32, a functional variant or a functional fragment thereof) and/or of the stl1 gene (referred to as stl1p and having, for example, the nucleic acid sequence of SEQ ID NO: 33, a functional variant or a functional fragment thereof). In an embodiment, the promoter is or comprises the tdh1p and/or the hor7p. In still another embodiment, the promoter comprises or consists essentially of the tdh1p and the hor7p. In a further embodiment, the promoter is the thd1p.

One or more promoters can be used to allow the expression of each heterologous polypeptides in the recombinant yeast host cell. In the context of the present disclosure, the expression “functional fragment of a promoter” when used in combination to a promoter refers to a shorter nucleic acid sequence than the native promoter which retain the ability to control the expression of the nucleic acid sequence encoding the heterologous protein or its chimera during the propagation phase of the recombinant yeast host cells. Usually, functional fragments are either 5′ and/or 3′ truncation of one or more nucleic acid residue from the native promoter nucleic acid sequence.

In some embodiments, the nucleic acid molecules include one or a combination of terminator sequence(s) to end the translation of the heterologous protein (or of the chimeric protein comprising same). The terminator can be native or heterologous to the nucleic acid sequence encoding the heterologous protein or its corresponding chimera. In some embodiments, one or more terminators can be used. In some embodiments, the terminator comprises the terminator derived from is from the dit1 gene (referred to as dit1t and can have, for example, the nucleic acid sequence of SEQ ID NO: 34, a functional variant or a functional fragment thereof), from the idp1 gene (referred to as idp1t and can have, for example, the nucleic acid sequence of SEQ ID NO: 35, a functional variant or a functional fragment thereof), from the gpm1 gene (referred to as gpm1t and can have, for example, the nucleic acid sequence of SEQ ID NO: 36, a functional variant or a functional fragment thereof), from the pma1 gene (referred to as pma1t and can have, for example, the nucleic acid sequence of SEQ ID NO: 37, a functional variant or a functional fragment thereof), from the tdh3 gene (referred to as tdh3t and can have, for example, the nucleic acid sequence of SEQ ID NO: 38, a functional variant or a functional fragment thereof), from the hxt2 gene (referred to as hxt2t and can have, for example, the nucleic acid sequence of SEQ ID NO: 39, a functional variant or a functional fragment thereof), from the adh3 gene (referred to as adh3t and can have, for example, the nucleic acid sequence of SEQ ID NO: 70, a functional variant or a functional fragment thereof), and/or from the ira2 gene (referred to as ira2t and can have, for example, the nucleic acid sequence of SEQ ID NO: 40, a functional variant or a functional fragment thereof). In an embodiment, the terminator comprises or is derived from the dit1 gene (referred to as dit1t and can have, for example, the nucleic acid sequence of SEQ ID NO: 34, a functional variant or a functional fragment thereof). In another embodiment, the terminator comprises or is derived from the adh3 gene (and can have, for example, the nucleic acid sequence of SEQ ID NO: 70, a functional variant or a functional fragment thereof). In the context of the present disclosure, the expression “functional variant of a terminator” refers to a nucleic acid sequence that has been substituted in at least one nucleic acid position when compared to the native terminator which retain the ability to end the expression of the nucleic acid sequence coding for the heterologous protein or its corresponding chimera. In the context of the present disclosure, the expression “functional fragment of a terminator” refers to a shorter nucleic acid sequence than the native terminator which retain the ability to end the expression of the nucleic acid sequence coding for the heterologous protein or its corresponding chimera.

In some embodiments, the heterologous nucleic acid molecules include one or a combination of signal sequence(s) allowing the export of the heterologous protein (or of the chimeric protein comprising same) outside the yeast host cell's wall. The signal sequence can simply be added to the nucleic acid molecule (usually in frame with the sequence encoding the heterologous protein) or replace the signal sequence already present in the heterologous protein. The signal sequence can be native or heterologous to the nucleic acid sequence encoding the heterologous protein or its corresponding chimera. In some embodiments, one or more signal sequences can be used. In some embodiments, the signal sequence is from the gene encoding the invertase protein (and can have, for example, the amino acid sequence of SEQ ID NO: 68, be a variant of the amino acid sequence of SEQ ID NO: 68 or be a fragment of the amino acid sequence of SEQ ID NO: 68), the AGA2 protein (and can have, for example, the amino acid sequence of SEQ ID NO: 69, be a variant of the amino acid sequence of SEQ ID NO: 69 or be a fragment of the amino acid sequence of SEQ ID NO: 69) or the fungal amylase (and can have, for example, the amino acid sequence of SEQ ID NO: 107, be a variant of the amino acid sequence of SEQ ID NO: 107 or be a fragment of the amino acid sequence of SEQ ID NO: 107). In the context of the present disclosure, the expression “functional variant of a signal sequence” refers to a nucleic acid sequence that has been substituted in at least one nucleic acid position when compared to the native signal sequence which retain the ability to direct the expression of the heterologous food and/or feed enzyme or its corresponding chimera outside the cell. In the context of the present disclosure, the expression “functional fragment of a signal sequence” refers to a shorter nucleic acid sequence than the native signal sequence which retain the ability to direct the expression of the heterologous food and/or feed enzyme or its corresponding chimera outside the cell.

In some embodiments in which it is desirable to express the heterologous protein inside the recombinant yeast host cell, the heterologous nucleic acid molecule can exclude the portion coding for the signal sequence which is found in the native gene encoding the food and/or feed enzyme.

The heterologous nucleic acid molecule encoding the heterologous protein, chimera, variant or fragment thereof can be integrated in the genome of the yeast host cell. The term “integrated” as used herein refers to genetic elements that are placed, through molecular biology techniques, into the genome of a host cell. For example, genetic elements can be placed into the chromosomes of the host cell as opposed to in a vector such as a plasmid carried by the host cell. Methods for integrating genetic elements into the genome of a host cell are well known in the art and include homologous recombination. The heterologous nucleic acid molecule can be present in one or more copies in the yeast host cell's genome. Alternatively, the heterologous nucleic acid molecule can be independently replicating from the yeast's genome. In such embodiment, the nucleic acid molecule can be stable and self-replicating.

The present disclosure also provides nucleic acid molecules for modifying the yeast host cell so as to allow the expression of the heterologous proteins, chimeras, variants or fragments thereof. The nucleic acid molecule may be DNA (such as complementary DNA, synthetic DNA or genomic DNA) or RNA (which includes synthetic RNA) and can be provided in a single stranded (in either the sense or the antisense strand) or a double stranded form. The contemplated nucleic acid molecules can include alterations in the coding regions, non-coding regions, or both. Examples are nucleic acid molecule variants containing alterations which produce silent substitutions, additions, or deletions, but do not alter the properties or activities of the encoded heterologous proteins, chimeras, variants or fragments.

In some embodiments, the heterologous nucleic acid molecules which can be introduced into the recombinant host cells are codon-optimized with respect to the intended recipient recombinant yeast host cell. As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

The heterologous nucleic acid molecules can be introduced in the yeast host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The present disclosure also provides nucleic acid molecules that are hybridizable to the complement nucleic acid molecules encoding the heterologous polypeptides as well as variants or fragments. A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified, e.g., in Sambrook, J., Fritsch, E. F. and Maniatis, T. MOLECULAR CLONING: A LABORATORY MANUAL, Second Edition, Cold Spring Harbor Laboratory Press, Cold Spring Harbor (1989), particularly Chapter 11 and Table 11.1 therein. The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. For more stringent conditions, washes are performed at higher temperatures in which the washes are identical to those above except for the temperature of the final two 30 min washes in 0.2×SSC, 0.5% SDS are increased to 60° C. Another set of highly stringent conditions uses two final washes in 0.1×SSC, 0.1% SDS at 65° C. An additional set of highly stringent conditions are defined by hybridization at 0.1×SSC, 0.1% SDS, 65° C. and washed with 2×SSC, 0.1% SDS followed by 0.1×SSC, 0.1% SDS.

Hybridization requires that the two nucleic acid molecules contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived. For hybridizations with shorter nucleic acids, i.e., oligonucleotides, the position of mismatches becomes more important, and the length of the oligonucleotide determines its specificity. In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

Yeast Compositions and Processes for Making Yeast Compositions

As indicated herein, the present disclosure allows for making a yeast composition from the recombinant yeast host cell of the present disclosure. In an embodiment, the yeast composition comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 weight % or more of the heterologous protein when compared to the total proteins of the yeast composition. In a specific embodiment, the yeast composition comprises at least 0.2 weight % of the heterologous protein when compared to the total proteins of the yeast composition. In another embodiment, the yeast composition comprises at least 0.001, 0.002, 0.003, 0.004, 0.005, 0.006, 0.007, 0.008, 0.009, 0.01, 0.011, 0.012, 0.013, 0.014, 0.015, 0.016, 0.017, 0.018, 0.019, 0.02, 0.021, 0.022, 0.023, 0.024, 0.025 g or more of the heterologous protein/g of the total proteins of the recombinant yeast host cell. In a specific embodiment, the yeast composition comprises at least 0.02 g of the heterologous protein/g of the total proteins of the recombinant yeast host cell. In yet another embodiment, the yeast composition comprises at least 0.05, 0.075, 0.1, 0.2, 0.3, 0.4, 0.5, 0.6, 0.7, 0.8, 0.9, 1.0, 1.1, 1.2, 1.3, 1.4, 1.5 weight % or more of the heterologous protein when compared to the total weight of the recombinant yeast host cell. In yet another embodiment, the yeast composition comprises at least 0.1 weight % of the heterologous protein when compared to the total weight of the recombinant yeast host cell. In still a further embodiment, the yeast composition comprises at least 0.0005, 0.0006, 0.0007, 0.0008, 0.0009, 0.001, 0.0011, 0.0012, 0.0013, 0.0014, 0.0015, 0.0016, 0.0017, 0.0018, 0.0019, 0.002, 0.003, 0.004, 0.005, 0.006, 0.007, 0.008, 0.009, 0.01 g or more of the heterologous protein when compared to the dry weight of the recombinant yeast host cell. In a specific embodiment, the yeast composition comprises at least 0.0011 g of the heterologous protein/g (dry weight) of the recombinant yeast host cell. In an embodiment in which the recombinant yeast host cell is formulated as a yeast cream, the yeast cream comprises at least 45, 46, 47, 48, 49, 50, 50.2, 51, 52, 53, 54, 55 weight % or more of the heterologous protein when compared to the total weight of the yeast cream. In a specific embodiment, the yeast cream comprises at least 50.2 weight % of the heterologous protein when compared to the total weight of the yeast cream. Such embodiments reduces or waives the requirement of supplementing the yeast composition with an exogenous protein (such as an exogenous enzyme) in a subsequent fermentation step. In another embodiment in which the heterologous protein is an heterologous enzyme, the present disclosure provides processes as well as yeast compositions having a specific minimal enzymatic activity and/or a specific range of enzymatic activity. For example, the yeast composition can comprise a minimal amount of enzymatic activity which can provide a minimal enzymatic activity/g dry cell weight, which can be, for example, at least 150, 200, 250, 300, 350, 400, 450, 500, 550, 600, 650, 700, 750, 800, 850, 900, 950, 1000, 2000, 3000, 4000, 5000, 6000, 7000, 8000 or more enzymatic activity units/g of dry cell weight. In a specific embodiment, the yeast composition can comprise a minimal amount of enzymatic activity of at least about 300 enzymatic activity units/g dry cell weight. Alternatively or in combination, the yeast composition can provide a minimal enzymatic activity/g or mg of the total protein of the recombinant yeast host cell, which can be, for example, 50, 100, 150, 200, 250, 300, 350, 400, 450, 500, 550, 600, 650, 700, 750, 800, 850, 900, 950 or more enzymatic activity units/g or mg of total proteins of the recombinant yeast host cell. In a specific embodiment, the yeast composition can comprise a minimal amount of enzymatic activity of at least about 200 enzymatic activity units/g or mg of total proteins of the recombinant yeast host cell. In another example, when the heterologous enzyme is an amylase such as a maltogenic amylase, the yeast composition can comprises a minimal amount of maltogenic amylase activity (for example, measured as MANU/g of dry weight of the yeast composition) which can be, for example, at least about 150, 200, 250, 300, 350, 400, 450, 500, 550, 600, 650, 700, 750, 800, 850, 900, 950, 1000, 2000, 3000, 4000, 5000, 6000, 7000, 8000 or more MANU/g of dry cell weight or 50, 100, 150, 200, 250, 300, 350, 400, 450, 500, 550, 600, 650, 700, 750, 800, 850, 900, 950 or more MANU/g of total proteins of the recombinant yeast host cell. In a specific embodiment, the yeast composition can comprise a minimal amount of maltogenic amylase activity (for example, measured as MANU/g of dry weight of the yeast composition) which can be at least about 1000 MANU/g of dry cell weight of total proteins of the recombinant yeast host cell. In still another example, when the heterologous enzyme is an amylase such as a glucoamylase, the yeast composition can comprise a minimal amount of glucoamylase activity (for example, measured as units of glucoamylase activity/g of dry weight of the yeast composition), which can be, for example, 150, 200, 250, 300, 350, 400, 450, 500, 550, 600, 650, 700, 750, 800, 850, 900, 950, 1000, 2000, 3000, 4000, 5000, 6000, 7000, 8000 or more glucoamylase units/g of dry cell weight. For example, when the heterologous enzyme is an amylase such as a glucoamylase, the yeast composition can comprise a minimal amount of 300 glucoamylase units/g of dry cell weight. In a further embodiment, when the heterologous enzyme is an amylase such as an alpha-amylase, the yeast composition can comprise a minimal amount of alpha-amylase activity (for example, measured as units of alpha-amylase activity/g of dry weight of the yeast composition), which can be, for example, 150, 200, 250, 300, 350, 400, 450, 500, 550, 600, 650, 700, 750, 800, 850, 900, 950, 1000, 2000, 3000, 4000, 5000, 6000, 7000, 8000 or more alpha-amylase units/g of dry cell weight. For example, when the heterologous enzyme is an amylase such as an alpha-amylase, the yeast composition can comprise a minimal amount of 300 alpha-amylase units/g of dry cell weight. In still another embodiment, when the heterologous enzyme is a phosphatase such as a phytase, the yeast composition can comprise a minimal amount of phytase activity (for example, measured as units of phytase activity/g of dry weight of the yeast composition), which can be, for example, 150, 200, 250, 300, 350, 400, 450, 500, 550, 600, 650, 700, 750, 800, 850, 900, 950, 1000, 2000, 3000, 4000, 5000, 6000, 7000, 8000 or more phytase activity units/g of dry cell weight. For example, when the heterologous enzyme is a phosphatase such as a phytase, the yeast composition can comprise a minimal amount of phytase activity of 300 phytase activity units/g of dry cell weight. In still another example, when the heterologous enzyme is an oxidase such as a glucose oxidase, the yeast composition can comprise a minimal amount of glucose oxidase activity (for example, measured as units of glucose oxidase activity/g of dry weight of the yeast composition), which can be, for example, 150, 200, 250, 300, 350, 400, 450, 500, 550, 600, 650, 700, 750, 800, 850, 900, 950, 1000, 2000, 3000, 4000, 5000, 6000, 7000, 8000 or more glucose oxidase activity units/g of dry cell weight. For example, when the heterologous enzyme is an oxidase such as a glucose oxidase, the yeast composition can comprise a minimal amount of 300 glucose oxidase activity units/g of dry cell weight.

The process for making the yeast composition broadly comprises two steps: a first step of propagating the recombinant yeast host cell and a second step of formulating the yeast composition.

The propagation can be conducted according to a traditional baker's yeast production method with a recombinant yeast host cell as described herein. The process is advantageous as it allows the expression of an heterologous protein (which has not physiological benefit to the recombinant yeast host cell) to levels at least similar (within an order of magnitude) to those of homologous proteins that are expressed natively in the recombinant yeast host cell (such as, for example, the invertase protein, which can be present, in some embodiments, as 11700 U/g when measured in dried yeast cream; specific activity 2900 U/mg enzyme, of total proteins; 0.004 g/g of dry weight of the recombinant yeast host cell; 0.4 weight % when compared to the weight of the recombinant yeast host cell; 50.2% weight % in a yeast cream; 0.008 g/g of total proteins; 0.8 weight % of the total proteins as indicated in Gascon, 1968). The propagation process can be a continuous method, a batch method or a fed-batch method. The (culture) medium can comprise a carbon source (such as, for example, molasses, sucrose, glucose, dextrose syrup, ethanol, corn, glycerol, corn steep liquor and/or a lignocellulosic biomass), a nitrogen source (such as, for example, ammonia or another inorganic source of nitrogen) and a phosphorous source (such as, for example, phosphoric acid or another inorganic source of phosphorous). The culture medium can further comprises additional micronutrients such as vitamins and/or minerals to support the propagation of the recombinant yeast host cell.

In the propagation process, the recombinant yeast host cell is placed in a culture medium which can, in some embodiments, allow for a specific growth rate of 0.25, 0.24, 0.23, 0.22, 0.21, 0.20, 0.19, 0.18, 0.17, 0.16 or 0.15 h⁻¹ or less. In order to limit the growth rate of the recombinant yeast host cell, in some embodiments, the process can further comprise controlling the addition of nutriments, such as carbohydrates. Limiting the growth rate of the recombinant yeast host cell during propagation can be achieved by maintaining the concentration of carbohydrates below 0.1, 0.01, 0.001 or 0.0001 weight % with respect to the volume of the culture medium. Controlling the concentration of the carbohydrates of the culture medium can be done by various means known in the art and can involve sampling the culture medium at various intervals, determining the carbohydrate concertation, alcohol concentration and/or gas (CO₂) concentration in those samples and adding or refraining from adding, if necessary additional carbohydrates in the culture medium to accelerate or decelerate the growth of the recombinant yeast host cell. In some embodiments, the process provides for adding nitrogen and/or phosphorous to match/support the growth rate of the recombinant yeast host cell.

The propagation process is preferably conducted under high aeration conditions. For example, in some embodiments, the process can include controlling the aeration of the vessel to achieve a specific aeration rate, for example, of at least 0.2, 0.3, 0.4, 0.5, 0.6, 0.7, 0.8, 0.9, 1.0, 1.1, 1.2 or 1.3 air volume/vessel volume/minute.

The propagation process can be conducted at a specific pH and/or a specific temperature which is optimal for the expression of the heterologous protein. As such, in embodiments in which the yeast is from the genus Saccharomyces, the process can comprise controlling the pH of the culture medium to between about 3.0 to about 6.0, about 3.5 to about 5.5 or about 4.0 to about 5.5. In a specific embodiment, the pH is controlled at about 4.5. In another example, in embodiments in which the yeast is from the genus Saccharomyces, the process can comprise controlling the temperature of the culture medium between about 20° C. to about 40° C., about 25° C. to about 30° C. or about 30° C. to about 35° C. In a specific embodiment, the temperature is controlled at between about 30° C. to about 35° C. (32° C. for example).

At the end of the propagation process, a specific concentration can be sought or achieved. In some embodiments, the concentration of the propagated recombinant yeast host cell in the culture medium is at least about 0.1, 0.15, 0.2, 0.25, 0.3, 0.35, 0.4, 0.45, 0.5, 1.0, 1.5, 2.0, 2.5, 3.0, 3.5, 4.0, 4.5, 5.0 or more weight % with respect to the volume of the culture medium. In a specific embodiment in which the recombinant yeast host cell is propagated using a fed-batch process, the concentration of the propagated recombinant yeast host cell in the culture medium is at least about 0.25 weight % with respect to the volume of the culture medium.

In the formulating step, the mixture obtained after propagation (comprising the propagated recombinant yeast host cell(s)) is modified to provide a yeast composition. One of the advantages of the recombinant yeast host cells of the present disclosure is that the heterologous protein is associated with the recombinant yeast host cell that therefore concentrating the biomass after propagation will also increase the amount/activity of the heterologous protein. In an embodiment for providing a yeast composition, at least one component of the mixture obtained after propagation is removed from the culture medium to provide the yeast composition. This component can be, without limitation, water, amino acids, peptides and proteins, nucleic acid residues and nucleic acid molecules, cellular debris, fermentation products, etc. In an embodiment, the formulating step comprises substantially isolating the propagated yeast recombinant host cells (e.g., the biomass) from the components of the culture medium. As used in the context of the present disclosure, the expression “substantially isolating” refers to the removal of the majority of the components of the culture medium from the propagated recombinant yeast host cells. In some embodiments, “substantially isolating” refers to concentrating the propagated recombinant yeast host cell to at least 5, 10, 15, 20, 25, 30, 35, 45% or more when compared to the concentration of the recombinant yeast host cell prior to the isolation. In order to provide the yeast composition, the propagated recombinant yeast host cells can be centrifuged (and the resulting cellular pellet comprising the propagated recombinant yeast host cells can optionally be washed), filtered and/or dried (optionally using a vacuum-drying technique). The isolated recombinant yeast host cells can then be formulated in a yeast composition. The formulation step can, in some embodiments, preserve the viability (at least in part) of the recombinant yeast host cells. As such, the yeast composition can be provided in an active or a semi-active form. The yeast composition can be provided in a liquid, semi-solid or dry form. In an embodiment, the yeast composition can be provided in the form of a cream yeast.

Yeast Products and Processes for Making Yeast Products

The recombinant yeast host cell of the present disclosure can be used in the preparation of a yeast composition for ultimately making a yeast product. In an embodiment, the yeast product comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 weight % or more of the heterologous protein when compared to the total proteins of the yeast product. In a specific embodiment, the yeast product comprises at least 0.2 weight % of the heterologous protein when compared to the total proteins of the yeast product. In another embodiment, the yeast product comprises at least 0.001, 0.002, 0.003, 0.004, 0.005, 0.006, 0.007, 0.008, 0.009, 0.01, 0.011, 0.012, 0.013, 0.014, 0.015, 0.016, 0.017, 0.018, 0.019, 0.02, 0.021, 0.022, 0.023, 0.024, 0.025 g or more of the heterologous protein/g of the total proteins of the recombinant yeast host cell. In a specific embodiment, the yeast product comprises at least 0.02 g of the heterologous protein/g of the total proteins of the recombinant yeast host cell. In yet another embodiment, the yeast product comprises at least 0.05, 0.075, 0.1, 0.2, 0.3, 0.4, 0.5, 0.6, 0.7, 0.8, 0.9, 1.0, 1.1, 1.2, 1.3, 1.4, 1.5 weight % or more of the heterologous protein when compared to the total weight of the recombinant yeast host cell. In yet another embodiment, the yeast product comprises at least 0.1 weight % of the heterologous protein when compared to the total weight of the recombinant yeast host cell. In still a further embodiment, the yeast product comprises at least 0.0005, 0.0006, 0.0007, 0.0008, 0.0009, 0.001, 0.0011, 0.0012, 0.0013, 0.0014, 0.0015, 0.0016, 0.0017, 0.0018, 0.0019, 0.002, 0.003, 0.004, 0.005, 0.006, 0.007, 0.008, 0.009, 0.01 g or more of the heterologous protein when compared to the dry weight of the recombinant yeast host cell. In a specific embodiment, the yeast product comprises at least 0.0011 g of the heterologous protein/g (dry weight) of the recombinant yeast host cell. Such embodiments reduces or waives the requirement of supplementing the yeast product with an exogenous protein (such as an exogenous enzyme) in a subsequent fermentation step. In another embodiment in which the heterologous protein is an heterologous enzyme, the present disclosure provides processes as well as yeast products having a specific minimal enzymatic activity and/or a specific range of enzymatic activity. For example, the yeast product can comprise a minimal amount of enzymatic activity which can provide a minimal enzymatic activity/g dry cell weight, which can be, for example, 150, 200, 250, 300, 350, 400, 450, 500, 550, 600, 650, 700, 750, 800, 850, 900, 950, 1000, 2000, 3000, 4000, 5000, 6000, 7000, 8000 or more enzymatic activity units/g of dry cell weight. In a specific embodiment, the yeast product can comprise a minimal amount of enzymatic activity of at least about 300 enzymatic activity units/g dry cell weight. Alternatively or in combination, the yeast product can provide a minimal enzymatic activity/g or mg of the total protein of the recombinant yeast host cell, which can be, for example, 50, 100, 150, 200, 250, 300, 350, 400, 450, 500, 550, 600, 650, 700, 750, 800, 850, 900, 950 or more enzymatic activity units/g of total proteins of the recombinant yeast host cell. In a specific embodiment, the yeast product can comprise a minimal amount of enzymatic activity of at least about 200 enzymatic activity units/g of total proteins of the recombinant yeast host cell. In another embodiment, when the heterologous enzyme is an amylase such as a maltogenic amylase, the yeast product can comprises a minimal amount of maltogenic amylase activity (for example, measured as MANU/g of dry weight of the yeast product) which can be, for example, at least about 150, 200, 250, 300, 350, 400, 450, 500, 550, 600, 650, 700, 750, 800, 850, 900, 950, 1000, 2000, 3000, 4000, 5000, 6000, 7000, 8000 or more MANU/g of dry cell weight or 50, 100, 150, 200, 250, 300, 350, 400, 450, 500, 550, 600, 650, 700, 750, 800, 850, 900, 950, 1000, 1250, 1500, 1750, 2000 or more MANU/g of total proteins of the recombinant yeast host cell. For example, the yeast product can comprises a minimal amount of maltogenic amylase activity (for example, measured as MANU/g of dry weight of the yeast product) which can be at least about 1000 MANU/g of dry cell weight of the recombinant yeast host cell. In still another embodiment, when the heterologous enzyme is an amylase such as a glucoamylase, the yeast product can comprise a minimal amount of glucoamylase activity (for example, measured as units of glucoamylase activity/g of dry weight of the yeast product), which can be, for example, 150, 200, 250, 300, 350, 400, 450, 500, 550, 600, 650, 700, 750, 800, 850, 900, 950, 1000, 2000, 3000, 4000, 5000, 6000, 7000, 8000 or more glucoamylase units/g of dry cell weight. For example, when the heterologous enzyme is an amylase such as a glucoamylase, the yeast product can comprise a minimal amount of 300 glucoamylase units/g of dry cell weight. In a further embodiment, when the heterologous enzyme is an amylase such as an alpha-amylase, the yeast product can comprise a minimal amount of alpha-amylase activity (for example, measured as units of alpha-amylase activity/g of dry weight of the yeast product), which can be, for example, 150, 200, 250, 300, 350, 400, 450, 500, 550, 600, 650, 700, 750, 800, 850, 900, 950, 1000, 2000, 3000, 4000, 5000, 6000, 7000, 8000 or more alpha-amylase units/g of dry cell weight. For example, when the heterologous enzyme is an amylase such as an alpha-amylase, the yeast product can comprise a minimal amount of 300 alpha-amylase units/g of dry cell weight. In still another embodiment, when the heterologous enzyme is a phosphatase such as a phytase, the yeast product can comprise a minimal amount of phytase activity (for example, measured as units of phytase activity/g of dry weight of the yeast product), which can be, for example, 150, 200, 250, 300, 350, 400, 450, 500, 550, 600, 650, 700, 750, 800, 850, 900, 950, 1000, 2000, 3000, 4000, 5000, 6000, 7000, 8000 or more phytase activity units/g of dry cell weight. For example, when the heterologous enzyme is a phosphatase such as a phytase, the yeast product can comprise a minimal amount of phytase activity of 300 phytase activity units/g of dry cell weight. In still another embodiment, when the heterologous enzyme is an oxidase such as a glucose oxidase, the yeast product can comprise a minimal amount of glucose oxidase activity (for example, measured as units of glucose oxidase activity/g of dry weight of the yeast product), which can be, for example, 150, 200, 250, 300, 350, 400, 450, 500, 550, 600, 650, 700, 750, 800, 850, 900, 950, 1000, 2000, 3000, 4000, 5000, 6000, 7000, 8000 or more glucose oxidase activity units/g of dry cell weight. For example, when the heterologous enzyme is an oxidase such as a glucose oxidase, the yeast product can comprise a minimal amount of glucose oxidase activity of 300 glucose oxidase activity units/g of dry cell weight.

In another embodiment in which the heterologous protein is an heterologous enzyme, the present disclosure provides processes as well as yeast products having a specific minimal enzymatic activity and/or a specific range of enzymatic activity. Advantageously, the cell-associated heterologous protein present in the yeast composition can be concentrated during processing and can remains biologically active to perform its intended function in the yeast products.

The process for making the yeast product broadly comprises two steps: a first step of providing propagated recombinant yeast host cells (which can, for example, be obtained by the process for making a yeast composition as indicated herein) and a second step of lysing the propagated yeast host cells. The process can include an optional separating step and an optional drying step.

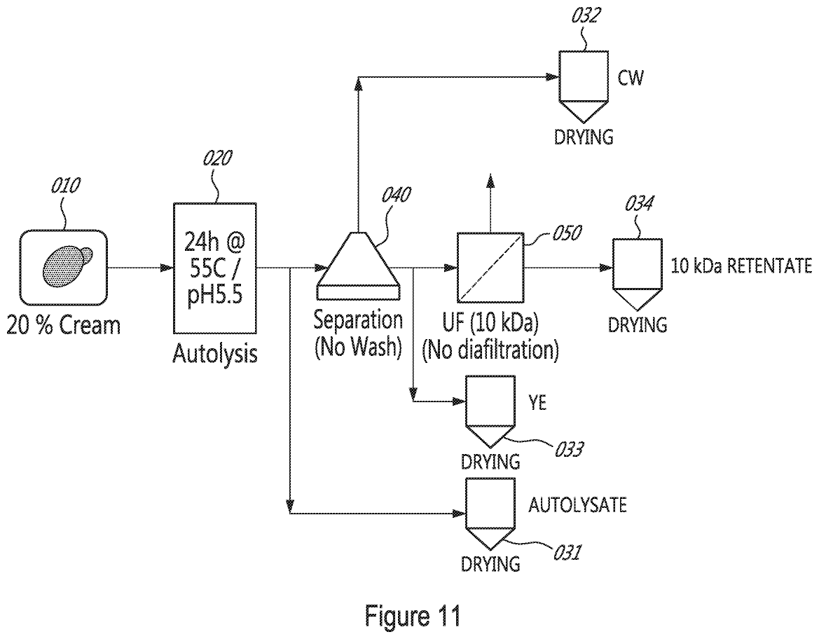

An embodiment of the process for making the yeast product is shown on FIG. 11. At step 010, propagated recombinant host cells are provided. In the embodiment shown on FIG. 11, the propagated recombinant host cells are provided as a 20% cream yeast even though additional embodiments of the propagated recombinant host cells can be provided (not shown on FIG. 11). Then, at step 020, the propagated recombinant yeast host cells are lysed to provide lysed recombinant yeast host cells. For example, the cells can be lysed using autolysis (which can be optionally be performed in the presence of additional exogenous enzymes) or homogenized (for example using a bead-milling technique). In an embodiment, the propagated recombinant yeast host cells can be lysed using autolysis. In the embodiment shown on FIG. 11, the propagated recombinant cells can be submitted to a combined heat and pH treatment for a specific amount of time (e.g., 24 h) in order to cause the autolysis of the propagated recombinant yeast host cells to provide the lysed recombinant yeast host cells. For example, the propagated recombinant cells can be submitted to a temperature of between about 40° C. to about 70° C. or between about 50° C. to about 60° C. The propagated recombinant cells can be submitted to a temperature of at least about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C. or 70° C. Alternatively or in combination the propagated recombinant cells can be submitted to a temperature of no more than about 70° C., 69° C., 68° C., 67° C., 66° C., 65° C., 64° C., 63° C., 62° C., 61° C., 60° C., 59° C., 58° C., 57° C., 56° C., 55° C., 54° C., 53° C., 52° C., 51° C., 50° C., 49° C., 48° C., 47° C., 46° C., 45° C., 44° C., 43° C., 42° C., 41° C. or 40° C. In another example, the propagated recombinant cells can be submitted to a pH between about 4.0 and 8.5 or between about 5.0 and 7.5. The propagated recombinant cells can be submitted to a pH of at least about, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4 or 8.5. Alternatively or in combination, the propagated recombinant cells can be submitted to a pH of no more than 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3., 5.2, 5.1, 5.0, 4.9, 4.8, 4.7, 4.6 or 4.5.

The process can also include a drying step. The drying step can include, for example, with spray-drying and/or fluid-bed drying. This step is shown as step 031, 032, 033 or 034 on FIG. 11. When the yeast product is an autolysate, the process includes directly drying the lysed recombinant yeast host cells after the lysis step without performing an additional separation of the lysed mixture. The direct drying step after the lysis step is shown as step 031 on FIG. 11.

To provide additional yeast products, it may be necessary to further separate the components of the lysed recombinant yeast host cells. For example, the cellular wall components (referred to as a “insoluble fraction”) of the lysed recombinant yeast host cell may be separated from the other components (referred to as a “soluble fraction) of the lysed recombinant yeast host cells. This separating step can be done, for example, by using centrifugation and/or filtration. The separation step is shown on FIG. 11 as step 040.

In the embodiment shown on FIG. 11, the insoluble fraction is not submitted to a washing step prior to the subsequent drying step 032 to provide the cell walls (“CW” on FIG. 11) as the yeast product or the subsequent drying step 033 to provide the yeast extract (“YE” on FIG. 11) as the yeast product. However, the process of the present disclosure can include one or more washing step(s) between steps 040 and 032 to provide the cell walls or between steps 040 and 033 to provide the yeast extract.

In an embodiment of the process, the insoluble fraction can be further separated prior to drying. For example, and as shown as step 050 on FIG. 11, the components of the soluble fraction having a molecular weight of more than 10 kDa can be separated out of the soluble fraction. This separation can be achieved, for example, by using filtration (and more specifically ultrafiltration). When filtration is used to separate the components, it is possible to filter out (e.g., remove) the components having a molecular weight less than about 10 kDa and retain the components having a molecular weight of more than about 10 kDa. The components of the soluble fraction having a molecular weight of more than 10 kDa can then optionally be dried, at step 034, to provide a retentate as the yeast product.

In the process described herein, the yeast product is provided as an inactive form. The yeast product can be provided in a liquid, semi-liquid or dry form.

In an embodiment, the process can also comprise substantially isolating/purifying the heterologous proteins from the yeast product. As used in the context of the present disclosure, the expression “substantially isolating/purifying the heterologous proteins from the lysed recombinant yeast host cells” refers to the removal of the majority of the components of the lysed recombinant yeast host cells from the heterologous proteins and providing same in an isolated/purified form. The heterologous protein can be provided in a liquid form or in a solid (dried) form. As such, the present disclosure provides an isolated heterologous protein obtainable or obtained by the process described herein. In an embodiment, the isolated heterologous protein is produced by a recombinant yeast host cell having and its signal sequenced has been swapped with a signal peptide from a protein naturally expressed in an heterologous organism, such as prokaryotes, a bacteria for example. In an alternative embodiment, a signal sequence has been added to the heterologous protein and this new signal sequence is from protein naturally expressed in prokaryotes such as bacteria.

The present invention will be more readily understood by referring to the following examples which are given to illustrate the invention rather than to limit its scope.

### Example I—Material and Methods

TABLE 1
Description of the yeast strains used in the examples. These strains were constructed with expression cassettes, integrated into
the FCY1 locus on each chromosome, the number of copies is provided in the table. The original strain background used for each
strain is also provided in the table. Each integrated cassette included a copy of an heterologous enzyme, one or more promoter
and one or more terminator. In some instances, the signal peptide of the heterologous enzyme has been replaced by another signal
peptide as indicated in the table. When the heterologous enzyme is expressed in a tethered form, the geometry in of the tether
is provided (see definition of formula I and II above) and the linker as well as the tether are provided. N.A. = not applicable.

| | | | Copies of | | | | | | |
|---|---|---|---|---|---|---|---|---|---|
| | | | heterologous | | | | | | |
| | | | enzyme | | | | | | |
| | Heterologous | Original | integrated | | | | | | |
| | enzyme | strain | per | | | Type of | Signal | | |
| Name | expressed | background | chromosome | Promoter | Terminator | expression | peptide¹ | Linker² | Tether³ |
| M2390 | None | N.A. | N.A. | N.A. | N.A. | N.A. | N.A. | N.A. | N.A. |
| (Saccharomyces | | | | | | | | | |
| cerevisiae) | | | | | | | | | |
| M8498 | Glucoamylase | M10474 | 1 | TEF2p | SED1t | Free | Invertase | None | None |
| | (SEQ ID | | | | | secreted | | | |
| | NO: 29) | | | | | | | | |
| M10074 | Alpha-amylase | M10474 | 1 | TEF2p | SED1t | Free | Invertase | None | None |
| | (SEQ ID | | | | | secreted | | | |
| | NO: 50) | | | | | | | | |
| M10474 | None | N.A. | N.A. | N.A. | N.A. | N.A. | N.A. | N.A. | N.A. |
| (Saccharomyces | | | | | | | | | |
| cerevisiae) | | | | | | | | | |
| M11312 | Phytase | M2390 | 1 | TEF2p | ADH3t | Free | Invertase | N.A. | N.A. |
| | (SEQ ID | | | | | secreted | | | |
| | NO: 67) | | | | | | | | |
| M12550 | None | N.A. | N.A. | N.A. | N.A. | N.A. | N.A. | N.A. | N.A. |
| (Saccharomyces | | | | | | | | | |
| cerevisiae) | | | | | | | | | |
| M12548 | None | N.A. | N.A. | N.A. | N.A. | N.A. | N.A. | N.A. | N.A. |
| (Saccharomyces | | | | | | | | | |
| boulardii) | | | | | | | | | |
| M12795 | Phytase | M12550 | 1 | TEF2p | ADH3t | Tethered - | Aga2 | (G₄S)₂ | Aga1/2 |
| | (SEQ ID | | | | | Formula (II) | | | (Aga2 |
| | NO: 67) | | | | | | | | on N- |
| | | | | | | | | | terminus |
| | | | | | | | | | of |
| | | | | | | | | | enzyme) |
| M12938 | Phytase | M12550 | 1 | TEF2p | ADH3t | Tethered - | Aga2 | (G₄S)₂ | Aga1/2 |
| | (SEQ ID | | | | | Formula (II) | | | |
| | NO: 67) | | | | | | | | |
| M12962 | None | N.A. | N.A. | N.A. | N.A. | N.A. | N.A. | N.A. | N.A. |
| (Saccharomyces | | | | | | | | | |
| cerevisiae var | | | | | | | | | |
| diastaticus) | | | | | | | | | |
| M13819 | Maltogenic | M10474 | 2 | TDH1p/HOR7p | DIT1t/IDP1t | Tethered - | Invertase | HA/G₄S | Spi1 |
| | alpha-amylase | | | | | Formula (I) | | | |
| | (SEQ ID | | | | | | | | |
| | NO: 51) | | | | | | | | |
| M13822 | Maltogenic | M10474 | 2 | TDH1p/HOR7p | DIT1t/IDP1t | Free | Invertase | None | None |
| | alpha-amylase | | | | | secreted | | | |
| | (SEQ ID | | | | | | | | |
| | NO: 51) | | | | | | | | |
| M13979 | Maltogenic | M10474 | 4 | TDH1p/HOR7p | DIT1t/IDP1t | Tethered - | Invertase | (G₄S)₂ | Spi1 |
| | alpha | | | | | formula (I) | | | |
| | amylase | | | | | | | | |
| | (SEQ ID | | | | | | | | |
| | NO: 51) | | | | | | | | |
| M14244 | Glucoamylase | M10474 | 1 | TEF2p | SED1t | Tethered - | Invertase | HA/G₄S | Sed1 |
| | (SEQ ID | | | | | formula (I) | | | |
| | NO: 29) | | | | | | | | |
| M14253 | Alpha-amylase | M10474 | 1 | TEF2p | SED1t | Tethered - | Invertase | HA/G₄S | Sed1 |
| | (SEQ ID | | | | | Formula (I) | | linker | |
| | NO: 50) | | | | | | | | |
| M14254 | Alpha-amylase | M10474 | 1 | TEF2p | SED1t | Tethered - | Invertase | None | Sed1 |
| | (SEQ ID | | | | | Formula (I) | | | |
| | NO: 50) | | | | | | | | |
| M14851 | Maltogenic | M10474 | 2 | TDH1p/HOR7p | DIT1t/IDP1t | Intracellular | N.A. | N.A. | N.A. |
| | alpha amylase | | | | | | | | |
| | (SEQ ID | | | | | | | | |
| | NO: 65) | | | | | | | | |
| M15215 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 84 |
| | NO: 72) | | | | | | | | |
| M15222 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 74 |
| | NO: 71) | | | | | | | | |
| M15532 | Maltogenic | M10474 | 2 | TDH1p/HOR7p | DIT1t/IDP1t | Intracellular | N.A. | N.A. | N.A. |
| | alpha amylase | | | | | | | | |
| | (SEQ ID | | | | | | | | |
| | NO: 108) | | | | | | | | |
| M15771 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 78 |
| | NO: 71) | | | | | | | | |
| M15772 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 82 |
| | NO: 71) | | | | | | | | |
| M15773 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 86 |
| | NO: 72) | | | | | | | | |
| M15774 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 76 |
| | NO: 71) | | | | | | | | |
| M15775 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 92 |
| | NO: 72) | | | | | | | | |
| M15776 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 88 |
| | NO: 72) | | | | | | | | |
| M15777 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 80 |
| | NO: 71) | | | | | | | | |
| M15778 | Alpha-amylase | M2390 | 1 | TEF2p | ADH3t | Tethered - | Invertase | SEQ ID | SEQ ID |
| | (SEQ ID | | | | | Formula (I) | | NO: 94 | NO: 74 |
| | NO: 71) | | | | | | | | |
| M15779 | Alpha-amylase | M2390 | 1 | TEF2p | ADH3t | Tethered - | Invertase | SEQ ID | SEQ ID |
| | (SEQ ID | | | | | Formula (I) | | NO: 95 | NO: 74 |
| | NO: 71) | | | | | | | | |
| M15780 | Alpha-amylase | M2390 | 1 | TEF2p | ADH3t | Tethered - | Invertase | SEQ ID | SEQ ID |
| | (SEQ ID | | | | | Formula (I) | | NO: 97 | NO: 74 |
| | NO: 71) | | | | | | | | |
| M15781 | Alpha-amylase | M2390 | 1 | TEF2p | ADH3t | Tethered - | Invertase | SEQ ID | SEQ ID |
| | (SEQ ID | | | | | Formula (I) | | NO: 98 | NO: 84 |
| | NO: 72) | | | | | | | | |
| M15782 | Alpha-amylase | M2390 | 1 | TEF2p | ADH3t | Tethered - | Invertase | SEQ ID | SEQ ID |
| | (SEQ ID | | | | | Formula (I) | | NO: 95 | NO: 84 |
| | NO: 72) | | | | | | | | |
| M15784 | Alpha-amylase | M2390 | 1 | TEF2p | ADH3t | Tethered - | Invertase | SEQ ID | SEQ ID |
| | (SEQ ID | | | | | Formula (I) | | NO: 93 | NO: 84 |
| | NO: 71) | | | | | | | | |
| M15783 | Alpha-amylase | M2390 | 1 | TEF2p | ADH3t | Tethered - | Invertase | SEQ ID | SEQ ID |
| | (SEQ ID | | | | | Formula (I) | | NO: 99 | NO: 74 |
| | NO: 71) | | | | | | | | |
| M15785 | Alpha-amylase | M2390 | 1 | TEF2p | ADH3t | Tethered - | Invertase | SEQ ID | SEQ ID |
| | (SEQ ID | | | | | Formula (I) | | NO: 93 | NO: 84 |
| | NO: 72) | | | | | | | | |
| M15786 | Alpha-amylase | M2390 | 1 | TEF2p | ADH3t | Tethered - | Invertase | SEQ ID | SEQ ID |
| | (SEQ ID | | | | | Formula (I) | | NO: 94 | NO: 84 |
| | NO: 72) | | | | | | | | |
| M15787 | Alpha-amylase | M2390 | 1 | TEF2p | ADH3t | Tethered - | Invertase | SEQ ID | SEQ ID |
| | (SEQ ID | | | | | Formula (I) | | NO: 96 | NO: 74 |
| | NO: 71) | | | | | | | | |
| M15788 | Alpha-amylase | M2390 | 1 | TEF2p | ADH3t | Tethered - | Invertase | SEQ ID | SEQ ID |
| | (SEQ ID | | | | | Formula (I) | | NO: 98 | NO: 74 |
| | NO: 71) | | | | | | | | |
| M16221 | Alpha-amylase | M2390 | 1 | TEF2p | ADH3t | Tethered - | Invertase | SEQ ID | SEQ ID |
| | (SEQ ID | | | | | Formula (I) | | NO: 97 | NO: 84 |
| | NO: 72) | | | | | | | | |
| M16222 | Alpha-amylase | M2390 | 1 | TEF2p | ADH3t | Tethered - | Invertase | SEQ ID | SEQ ID |
| | (SEQ ID | | | | | Formula (I) | | NO: 99 | NO: 84 |
| | NO: 72) | | | | | | | | |
| M16251 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 90 |
| | NO: 72) | | | | | | | | |
| M16252 | Alpha-amylase | M2390 | 1 | TEF2p | ADH3t | Tethered - | Invertase | SEQ ID | SEQ ID |
| | (SEQ ID | | | | | Formula (I) | | NO: 96 | NO: 84 |
| | NO: 72) | | | | | | | | |
| M16273 | Glucose | M10474 | 2 | TDH1p/HOR7p | DIT1t/IDP1t | Intracellular | N.A. | N.A. | N.A. |
| | oxidase | | | | | | | | |
| | (SEQ ID | | | | | | | | |
| | NO: 103) | | | | | | | | |
| M16540 | Fungal | M10474 | 2 | TDH1p/HOR7p | DIT1t/IDP1t | Free | Fungal | N.A. | N.A. |
| | amylase | | | | | secreted | amylase | | |
| | (SEQ ID | | | | | | | | |
| | NO: 105) | | | | | | | | |
| M16772 | Fungal | M10474 | 2 | TDH1p/HOR7p | DIT1t/IDP1t | Free | Invertase | N.A. | N.A. |
| | amylase | | | | | secreted | | | |
| | (SEQ ID | | | | | | | | |
| | NO: 105) | | | | | | | | |
| M16780 | Glucose | M10474 | 2 | TDH1p/HOR7p | DIT1t/IDP1t | Free | Invertase | N.A. | N.A. |
| | oxidase | | | | | secreted | | | |
| | (SEQ ID | | | | | | | | |
| | NO: 103) | | | | | | | | |
| T2633 | Phytase | M12548 | 1 | TEF2p | ADH3t | Free | Invertase | N.A. | N.A. |
| | (SEQ ID | | | | | secreted | | | |
| | NO: 66) | | | | | | | | |
| T2634 | Phytase | M12548 | 1 | TEF2p | ADH3t | Tethered - | Invertase | (G₄S)₂ | Sed1 |
| | (SEQ ID | | | | | Formula (I) | | | |
| | NO: 66) | | | | | | | | |
| T2635 | Phytase | M12548 | 1 | TEF2p | ADH3t | Tethered - | Invertase | (G₄S)₂ | Tir1 |
| | (SEQ ID | | | | | Formula (I) | | | |
| | NO: 66) | | | | | | | | |
| T2636 | Phytase | M12548 | 1 | TEF2p | ADH3t | Tethered - | Invertase | (G₄S)₂ | Cwp2 |
| | (SEQ ID | | | | | Formula (I) | | | |
| | NO: 66) | | | | | | | | |
| T2637 | Phytase | M12548 | 1 | TEF2p | ADH3t | Tethered - | Invertase | (G₄S)₂ | Spi1 |
| | (SEQ ID | | | | | Formula (I) | | | |
| | NO: 66) | | | | | | | | |
| T2638 | Phytase | M12548 | 1 | TEF2p | ADH3t | Tethered - | Invertase | (G₄S)₂ | Pst1 |
| | (SEQ ID | | | | | Formula (I) | | | |
| | NO: 66) | | | | | | | | |
| T2705 | Phytase | M2390 | 1 | TEF2p | ADH3t | Tethered - | Aga2 | (G₄S)₂ | Aga1/2 |
| | (SEQ ID | | | | | Formula (II) | | | |
| | NO: 67) | | | | | | | | |
| T2706 | Phytase | M2390 | 1 | TEF2p | ADH3t | Tethered - | Invertase | (G₄S)₂ | Aga1/2 |
| | (SEQ ID | | | | | Formula (I) | | | |
| | NO: 67) | | | | | | | | |
| T2816 | Phytase | M12550 | 1 | TEF2p | ADH3t | Tethered - | Invertase | (G₄S)₂ | Sed1 |
| | (SEQ ID | | | | | Formula (I) | | | |
| | NO: 67) | | | | | | | | |
| T2986 | Maltogenic | M10474 | 2 | TDH1p/HOR7p | DIT1t/IDP1t | Tethered - | Invertase | HA/(G₄S)₂ | Flo1 |
| | alpha-amylase | | | | | Formula (I) | | | |
| | (SEQ ID | | | | | | | | |
| | NO: 51) | | | | | | | | |
| T2987 | Maltogenic | M10474 | 2 | TDH1p/HOR7p | DIT1t/IDP1t | Tethered - | Invertase | HA/(G₄S)₂ | Sed1 |
| | alpha-amylase | | | | | Formula (I) | | | |
| | (SEQ ID | | | | | | | | |
| | NO: 51) | | | | | | | | |
| T2988 | Maltogenic | M10474 | 2 | TDH1p/HOR7p | DIT1t/IDP1t | Tethered - | Invertase | HA/(G₄S)₂ | Tir1 |
| | alpha-amylase | | | | | Formula (I) | | | |
| | (SEQ ID | | | | | | | | |
| | NO: 51) | | | | | | | | |
| T2989 | Maltogenic | M10474 | 2 | TDH1p/HOR7p | DIT1t/IDP1t | Tethered - | Invertase | HA-(G₄S)₂ | Cwp2 |
| | alpha-amylase | | | | | Formula (I) | | | |
| | (SEQ ID | | | | | | | | |
| | NO: 51) | | | | | | | | |
| T2990 | Maltogenic | M10474 | 2 | TDH1p/HOR7p | DIT1t/IDP1t | Tethered - | Invertase | HA/(G₄S)₂ | Ccw1 |
| | alpha-amylase | | | | | Formula (I) | | | |
| | (SEQ ID | | | | | | | | |
| | NO: 51) | | | | | | | | |
| T2991 | Maltogenic | M10474 | 2 | TDH1p/HOR7p | DIT1t/IDP1t | Tethered - | Invertase | HA/(G₄S)₂ | Spi1 |
| | alpha-amylase | | | | | Formula (I) | | | |
| | (SEQ ID | | | | | | | | |
| | NO: 51) | | | | | | | | |
| T2994 | Maltogenic | M10474 | 2 | TDH1p/HOR7p | DIT1t/IDP1t | Free | Invertase | None | None |
| | alpha-amylase | | | | | secreted | | | |
| | (SEQ ID | | | | | | | | |
| | NO: 51) | | | | | | | | |
| T3892 | Maltogenic | M10474 | 2 | TDH1p/HOR7p | DIT1t/IDP1t | Intracellular | N.A. | N.A. | N.A. |
| | alpha amylase | | | | | | | | |
| | (SEQ ID | | | | | | | | |
| | NO: 65) | | | | | | | | |
| T4328 | Maltogenic | M10474 | 2 | TDH1p/HOR7p | DIT1t/IDP1t | Tethered | Invertase | (G₄S)₂ | Spi1 |
| | alpha amylase | | | | | | | | |
| | (SEQ ID | | | | | | | | |
| | NO: 51) | | | | | | | | |
| T4329 | Maltogenic | M10474 | 2 | TDH1p/HOR7p | DIT1t/IDP1t | Free | Invertase | N.A. | N.A. |
| | alpha amylase | | | | | secreted | | | |
| | (SEQ ID | | | | | | | | |
| | NO: 51) | | | | | | | | |
| T4330 | Maltogenic | M10474 | 2 | TDH1p/HOR7p | DIT1t/IDP1t | Intracellular | N.A. | N.A. | N.A. |
| | alpha amylase | | | | | | | | |
| | (SEQ ID | | | | | | | | |
| | NO: 65) | | | | | | | | |
| T4336 | Maltogenic | M12962 | 2 | TDH1p/HOR7p | DIT1t/IDP1t | Tethered - | Invertase | (G₄S)₂ | Spi1 |
| | alpha amylase | | | | | Formula (I) | | | |
| | (SEQ ID | | | | | | | | |
| | NO: 51) | | | | | | | | |
| T4337 | Maltogenic | M12962 | 2 | TDH1p/HOR7p | DIT1t/IDP1t | Free | Invertase | N.A. | N.A. |
| | alpha amylase | | | | | secreted | | | |
| | (SEQ ID | | | | | | | | |
| | NO: 51) | | | | | | | | |
| T4338 | Maltogenic | M12962 | 2 | TDH1p/HOR7p | DIT1t/IDP1t | Intracellular | N.A. | N.A. | N.A. |
| | alpha amylase | | | | | | | | |
| | (SEQ ID | | | | | | | | |
| | NO: 65) | | | | | | | | |

¹Invertase = SEQ ID NO: 52, Aga2 = SEQ ID NO: 69, fungal amylase = SEQ ID NO: 107
²HA = SEQ ID NO: 53: (G₄S)₂ = SEQ ID NO: 54
³Flo1 tether is a transmembrane domain located at the C-terminus = SEQ ID NO: 10; Sed1 tether is a GPI anchor located at the C-terminus = SEQ ID NO: 12; Tir1 tether is a mannoprotein GPI fragment located at the C-terminus = SEQ ID NO: 14; Cwp2 tether is a mannoprotein GPI fragment located at the C-terminus = SEQ ID NO: 16; Ccw12 tether is a mannoprotein GPI fragment located at the C-terminus = SEQ ID NO: 18; Spi1 tether is a GPI anchor located at the C-terminus = SEQ ID NO: 20; Pst1 tether is a GPI anchor = SEQ ID NO: 22; Aga1/2 tether, Aga2 disulfide bond to Aga1; Aga1 has GPI anchor, the enzyme is fused to Aga2 at the C-terminus = SEQ ID NO: 24: Aga1/2 tether, Aga2 disulfide bond to Aga1; Aga1 has GPI anchor, the enzyme is fused to Aga2 at the N-terminus = SEQ ID NO: 26.

Cell growth. Cells were grown overnight in 5 mL YPD (10 g/L yeast extract, 20 g/L bacteriological peptone, 40 g/L glucose). One (1) mL of whole culture as harvested and cells were pelleted by centrifugation. Cell-free supernatant was removed and saved for later analysis. Cell pellet was washed once and resuspended in deionized water.

Cream yeast and inactivated cream yeast. After the fermentation, the harvested fermentation broth was centrifuged and washed using a laboratory scale separator (from GEA) to prepare yeast cream with a final dry weight close to 20%. To make the inactivated cream yeast, about 600 g of cream yeast was heated on a temperature controlled stirring/hot plate until 75° C. was reached. The cream was kept for 15 minutes at 75° C. and then removed from heat source.

Spray drying. Spray dried samples were prepared by drying at 150° C. with a mini spray dryer (Buchi B-290). Feeding rate was kept to maintain outlet temperature around 80-85° C.

Bead-milling/making bead-milled homogenate. Cream yeast was disrupted (with typical disruption efficiency of >95% of cells) by bead milling under the following bead mill conditions. Cream yeast (˜20% solids) was bead-milled with a Dyno KDL with 0.6 L chamber volume at 4° C., using 0.5-0.75 mm glass beads filling the chamber to 80% with 1.6 g/mL packing capacity and a 64 mm diameter agitator with peripheral speed of 10 m/s. The cream yeast flow rate was 6 kg/Uh.

Preparation of instant dried yeast (01). After the commercial fermentations targeting for the production of IDY samples, the harvested broth was centrifuged and washed using a laboratory scale GEA separator to prepare yeast cream with a final dry weight close to 20%. The cream was then filtered in a vacuum filtration system to make cake yeast. To remove additional water, the yeast cake was further pressed to achieve a dry weight of about 35% before extrusion. The pressed cake was then extruded after well mixed with span for 5 minutes. The span addition rate was 1% on yeast dry matter basis. After extrusion, the yeast was dried in a lab-scale fluidized-bed dryer (Aeromatic AG). The drying temperature was set and controlled at 35-40° C. The drying lasted about 20-25 minutes to achieve a solids content of more than 94%. In term of the fermentation recipes, the significant difference for the IDY fermentation recipe is that it has a 2 hrs maturation period towards end of the fermentation, in which ammonia (N) is stopped and fermentation temperature is increased to 35° C.

Fermenter autolysis. At least 3 L (minimum working volume) of cream at 20% solids was transferred into a 20 L fermenter (BiOENGiNEERiNG). Autolysis was performed at 55° C. and pH 5.5 (automated pH control with 2N sulfuric acid) with a gentle agitation at 70 rpm. Autolysate (˜20% dry weight) was harvested after a 24 hours incubation and separated as described below.

Lab scale autolysis. This autolysis is similar to the fermenter autolysis described above, but was performed at a smaller scale and with slightly different parameters. The cream yeast (20% solids) was submitted to autolysis and the pH was adjusted to pH 7. The mixture was incubated in 50 mL conical tubes in a 55° C. water bath for 48 hours.

Separation of autolysate without washing. After fermenter autolysis, the total autolysate was separated at 11,000 RCF for 10 minutes in 1 L bottles in a Sorvall Lynx 6000 centrifuge to obtain a soluble fraction (11-13% dry weight, yeast extract) and insoluble fraction (yeast cell wall). Dry weight and enzyme activity were measured for the total autolysate, yeast extract and cell wall fractions for dry weight and MANU balances.

Separation of autolysate with washing. Separations were performed by centrifuging fermenter autolysate in 50 mL conical tubes for 10 minutes at 3,000 RCF. Two additional washes were performed by adding water equal to the weight of supernatant obtained from the centrifuge step. YE (yeast extract) separation yield is calculated as the recovery of solids from separation only (WF=0) and of separation plus one or two washes (WF=1 or 2, respectively), relative to the starting solids in the autolysate. YE MANU recovery is calculated as the activity (in Phadebas MANU) from separation only (WF=0) and of separation plus one or two washes (WF=1 or 2), relative to starting total Phadebas MANU in the autolysate.

Ultrafiltration. Fermenter autolysate was separated by centrifuging in 1 L bottles at 11,000 RCF and the yeast extract fraction was further concentrated by ultrafiltration with a 10 kDa molecular weight cutoff PES membrane (Millipore, Biomax-10). The retentate fraction is retained by the membrane and permeate fraction passes through the membrane.

Maltogenic amylase assay. One Maltogenic Amylase Novo Unit, MANU, is the amount of enzyme which under standard conditions will cleave one micromol maltotriose per minute. Prior to assaying for enzymatic activity, cream yeast samples were inactivated by incubation at 60° C. for 10 minutes in MANU assay buffer (0.1 M citric acid, pH 5.0). Samples were then mixed with 20 mg/ml maltotriose substrate and incubated at 37° C. for 30 minutes. Reactions were stopped by addition of an equal volume of 1 N sodium hydroxide stop reagent. Glucose hydrolyzed by maltogenic amylase activity was measured after a 15-minute room temperature incubation with glucose (HK) assay reagent (Sigma G3293). Absorbance was read at 340 nm in a spectrophotometer. Unknown samples were compared to a dose curve of Novamyl® with known enzyme activity. This method was applied to generate the results of FIG. 1 only.

Phadebas MANU enzyme activity assay. Phadebas tablets contain a water insoluble starch substrate and a blue dye, bound to the dye with crosslinks. The substrate is hydrolyzed by maltogenic amylase, releasing blue dye which is soluble. After terminating the reaction and centrifuging, the absorbance of the solution was measured spectrophotometrically and is considered a proxy for enzyme activity. For each sample, one Phadebas tablet was added to 4.9 mL of citrate-phosphate buffer (70 mM disodium hydrogen phosphate, 30 mM citric acid, pH 5.5), incubated in a 60° C. water bath for 5 minutes. Then, 0.1 mL of standard or sample, diluted in citrate-phosphate buffer, was added to the tablet and buffer solution and incubated for 15 minutes in the 60° C. water bath. The reaction was terminated by adding 1 mL of 0.5 M sodium hydroxide solution and mixing. The tubes were centrifuged to remove solids and absorbance of the substrate was measured at 620 nm with a spectrophotometer. Samples (dry or liquid) are compared to a dose curve of Novamyl® with known activity. This methods was applied to generate all of the MANU results, except for FIG. 1.

Glucose oxidase assay. Cells were grown in batch in yeast extract peptone media plus 2% glucose at 30° C. for 24 hours. To obtain the disrupted washed cell supernatant, the cells were dead-beaten with glass beads 2×1 min in assay buffer, with one minute rest between. The supernatant was separated from the whole lysate by centrifugation. Whole culture, supernatant, disrupted washed cell supernatant (which reflects the intracellular cell-associated activity), washed cells or a positive control of Gluzyme® (2.40 GODU/mL corresponding to 10 000BG) were measured with the K-GLOX™ kit (Megazyme): samples in assay buffer (100 mM potassium phosphate, pH 7, containing 0.5 mg/mL BSA and 0.02% (w/v) sodium azide) were mixed with 90 mg/mL glucose and POD mixture and incubated at room temperature for 20 minutes. Absorbance was measured with a spectrophotometer at 510 nm.

Alpha-amylase assay (FIG. 3). Alpha-amylase activity was measured by adding 25 μL washed cells or cell free supernatant to 25 μL 5 mM p-Nitrophenyl α-D-hexaoside in 50 mM sodium acetate pH 5. The reaction was incubated at 35° C. for 2 hours and terminated by the addition of 50 μL 1M sodium bicarbonate. Cells were pelleted, 50 μL of the assay mixture was transferred to a microtiter plate and absorbance at 405 nm was measured. Activity of the cell fraction was represented as a percentage of the total activity (“bound”+“free”).

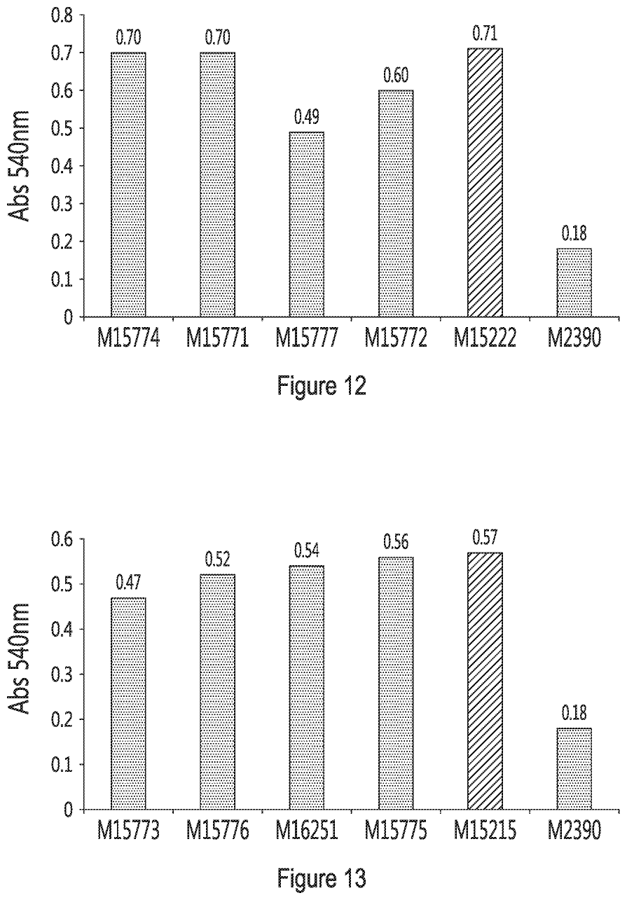

Alpha-amylase assay (FIGS. 12 to 15). The strains were initially grown in 600 μL of YPD40 at 35° C. for 48 h in 96-well plates on a shaker at 900 rpm. Alpha-amylase activity was determined by adding 25 μL of washed cells or cell-free supernatant to 100 μL of 1% raw starch with 50 mM sodium acetate buffer (pH 5.2). The assay was treated for 30 min at 85° C. using an Eppendorf Gradient Cycler. The reducing sugars were measured using the Dinitrosalicylic Acid Reagent Solution (DNS) method, using a 2:1 DNS:starch assay ratio and boiled at 100° C. for 5 min. The absorbance was measured at 540 nm.

Wheat starch activity assay. Cells were grown in batch in yeast extract peptone media plus 4% glucose at 35° C. for 48 hours. Whole culture, supernatant and washed cells resuspended in assay buffer (50 mM sodium acetate, pH 5) were mixed with 1% wheat starch in assay buffer and incubated at 60° C. for 5 minutes. Then, 3,5-dinitrosalicylic acid was added to react with reducing ends and boiled at 99° C. for 5 minutes. Absorbance was measured with a spectrophotometer at 540 nm.

Fungal amylase activity. Cells were grown in batch in yeast extract peptone media plus 2% glucose at 30° C. for 24 hours. Whole culture, supernatant, and either disrupted cell supernatant or washed cells were resuspended in assay buffer (70 mM disodium hydrogen phosphate, 30 mM citric acid, pH 5.5) were mixed with 1% gelatinized wheat starch in assay buffer and incubated at 30° C. for 1 hour. 3,5-Dinitrosalicylic acid (DNS) was added to react with reducing ends and boiled at 99° C. for 5 minutes. Absorbance was measured with a spectrophotometer at 540 nm.

Phytase activity assay. A 2-fold serial dilution of 1 M potassium phosphate monobasic was prepared as a standard for calculating FTUs. 190 μl of 5 mM sodium phytate solution pH 5.5 was added to each well of a 96 well PCR plate. Standards or supernatants of overnight cultures of yeast in yeast extract peptone media with 4% glucose were combined with 5 mM sodium phytate solution pH 5.5 and were incubated at 37° C. for 30 min. Cell associated samples were measured again following 2 hours of incubation. Equal volumes of reaction and color change solution (4 parts reagent A to 1 part reagent B, where reagent A is 12 mM ammonium heptamolybdate-HCl in water and reagent B is 2.7% ferrous sulfate in water) were combined and incubated for 10 minutes at room temperature before pelleting at 3500 rpm for 3 minutes. Absorbance of each sample or standard was read at 700 nm in a spectrophotometer.

SDS-PAGE. Equal volumes of sample and loading buffer (250 mM Tris, pH 6.8, 30% glycerol, 10% SDS, 0.1% bromophenol blue; with or without 50 mM DTT for reducing or non-reducing conditions, respectively) were mixed and heated at 100° C. for 2 minutes. 20 μl of each sample plus buffer was loaded on a 4-20% Tris glycine gel and run at 175V for 75 minutes. After electrophoresis, the gel was washed in water and stained with SimplyBlue SafeStain® to visualize total protein. Bio-rad All Blue® protein standard was used as a molecular weight reference.

### Example II—Expression of Cell-Associated Maltogenic Alpha-Amylases

The expression of heterologous MAA, especially in the presence of a tether, provided the recombinant yeasts with maltogenic amylase activity both in the cell pellet (FIG. 1A) and, at a larger scale, in the cream yeasts (FIG. 1B). In comparison, the corresponding wild-type strain failed to exhibit any maltogenic amylase activities (FIGS. 1A and 1B).

Results shown in FIG. 1 were obtained by expressing the heterologous MAA from the promoter of the tdh1 gene. Similar results were obtained when a combination of two promoters (from the tdh1 gene and the hor7 gene, data not shown). Enzymatic activity is higher in strains expressing the heterologous maltogenic amylase.

A yeast strain expressing and intracellular G. stearothermophilus MAA (M15532) was propagated by aerobic fed-batch on molasses and a yeast cream was then made. Its MANU activity was determined. As shown in Table 2, the heterologous enzyme is calculated to be 3.7% of total cellular protein.

TABLE 2
Calculations for maltogenic amylase as a percent of total
cell protein. Enzyme activity of cream yeast and of purified
enzyme was determined in a Phadebas enzyme assay with
comparison to a dose curve of the enzyme Novamyl ® standards
with known maltogenic amylase units (MANU).

| | |
|---|---|
| Percent protein in cream | 50.2% |
| Enzyme activity (MANU/g dry cell weight), measured | 16000 |
| after release by autolysis or homogenization | |
| Specific activity of pure enzyme (MANU/mg) | 872 |
| Specific activity of pure enzyme (MANU/g) | 872000 |
| Enzyme per gram dry weight (g/g) | 0.018 |
| Enzyme per total protein (g/g) | 0.037 |
| Enzyme as % of total protein | 3.7% |

Another strain expressing and intracellular G. stearothermophilus MAA (M14851) was propagated (fed batch on molasses) and MANU activity was determined. As shown in Table 3, in the untreated total broth, between 25.6 and 39.3 MANU activity was detected. After washing and concentrating the cream, between 112 and 288 MANU activity was detected.

TABLE 3
Concentrating yeast biomass concentrates cell-associated maltogenic
amylase. Enzyme activity was determined in Phadebas enzyme
assays with comparison to a dose curve of Novamyl standards
with known maltogenic amylase units (MANU).

Phadebas MANU/ml

| | | Washed and |
|---|---|---|
| | Total | concentrated |
| | broth | cream |
| M14851 | (~6% | (19-20% |
| propagation | solids) | solids) |
| I200617 | 25.6 | 112.2 |
| I210617 | 35.6 | 232.0 |
| I220617 | 39.3 | 287.6 |

Another strain expressing a tethered G. stearothermophilus MAA (M13879) was propagated (fed batch on molasses) and MANU activity was determined in various yeast preparations. The results are shown Table 4. Cream yeast activity data on 1 day after commercial propagation is the most representative measure of the cream in its original form. All other data were obtained on 8 days after the commercial propagation.

TABLE 4
Phadebas MANU activity per gram dry weight of various
preparations of M13979. Enzyme activity was determined
in Phadebas enzyme assays with comparison to a dose
curve of the enzyme Novamyl ® standards
with known maltogenic amylase units (MANU).

| | Phadebas MANU equivalent/ | |
|---|---|---|
| | gram dry weight | |
| | 1 day after | 8 days after |
| M13979 sample | propagation | propagation |
| Cream | 1087 | 3157 |
| Bead-milled homogenate | | 8698 |
| Cream, spray dried | | 1121 |
| Inactivated cream, spray dried | | 2039 |
| Bead-milled homogenate, spray dried | | 6721 |

MANU and wheat starch activity were determined in different preparations of a yeast strain expressing intracellularly the maltogenic alpha amylase from G. stearothermophilus (M15532) and propagated in fed-batch on molasses. The results are provided in Tables 5 to 9 showing the effects of the different preparations on the level of enzymatic activity observed.

TABLE 5
Phadebas and wheat starch enzyme assays to measure maltogenic
amylase activity on various M15532 preparations. Enzyme
activity was determined in Phadebas enzyme assays with
comparison to a dose curve of the enzyme Novamyl ® standards
with known maltogenic amylase units (MANU).

| | | | | Wheat |
|---|---|---|---|---|
| | | | Phadebas | starch |
| | | | MANU/ | MANU/ |
| M15532 | M15532 | | g dry | g dry |
| propagation | sample | Form | weight | weight |
| High protein | Untreated cream | Liquid | 287 | 574 |
| recipe | Bake lab autolyzed | Liquid | 17826 | 15328 |
| I060917 | cream (pH 7, 48 h, | | | |
| Mix of 4 | 55° C.) (liquid) | | | |
| propagations | Untreated cream | Liquid | 96 | |
| (I280817, | Bake lab autolyzed | Liquid | 23614 | 12920 |
| B300817, | cream (pH 7, 48 h, | | | |
| B310817, | 55° C.) (liquid) | | | |
| B300817) | Bead-milled | Liquid | 15916 | 12903 |
| | homogenate | | | |
| | (liquid) | | | |
| | Bead-milled | Dry | 10607 | 7764 |
| | homogenate | | | |
| | (spray dried) | | | |

TABLE 6
Activity results in cream, lab-scale autolyzed cream
(incubated 48 h at 55° C., pH 7) and rehydrated
instant dry yeast (IDY) samples. Enzyme activity was
determined in Phadebas enzyme assays with comparison
to a dose curve of the enzyme Novamyl ® standards
with known maltogenic amylase units (MANU).

| | | Phadebas | Phadebas |
|---|---|---|---|
| | % | MANU/ml | MANU/g |
| M15532 Sample | solids | of sample | DCW |
| Cream | 17.9 | 58 | 325 |
| Cream after 48 h, 55° C., pH 7 | 17.9 | 3572 | 19955 |
| 37° C. rehydrated IDY | 15.9 | 545 | 3438 |
| Cold shocked IDY | 15.4 | 454 | 2958 |

TABLE 7
Dry weight and enzyme activity balances in autolysate, yeast extract, ultrafiltration retentate and yeast cell wall preparations
before and after drying of different preparations of the yeast strain M15532. Enzyme activity was determined in Phadebas enzyme
assays with comparison to a dose curve of the enzyme Novamyl ® standards with known maltogenic amylase units (MANU).

| | | | | | | | | | | MANU | |
|---|---|---|---|---|---|---|---|---|---|---|---|
| | | | | | | | | | | BALANCE | |
| | BEFORE | | AFTER DRYING | | | | | | | PROCESS + | |
| | DRYING | | | | BAKE LAB | | NRC24NOV | | | DRYING | |
| | dw | MAA | dw | NRC10NOV | MANU/gdw | | MANU/gdw | | dwt | BAKE | |
| SAMPLE | % | MANU/gdw | % | MANU/gdw | AV | % RD | AV | % CV | BALANCE | LAB | NRC24NOV |
| AUTOLYSATE | 18.0 | 8983 | 91.3 | 13568 | 14812 | 6% | 16864 | 17% | 100 | 100 | 100 |
| YE | 11.6 | 14422 | 93.7 | 23067 | 27566 | 5% | 24028 | 14% | 47 | 87 | 66 |
| 10 kDa | 15.1 | 83332 | 93.7 | 25984 | 80817 | 5% | 75552 | 15% | 18 | 98 | 80 |
| RETENTATE | | | | | | | | | | | |
| CW | 37.0 | 883 | 94.1 | 700 | 3757 | 4% | 3421 | 4% | 53 | 14 | 11 |

TABLE 8
Results of separation of yeast extract from total autolysate
and enzyme recovery with and without washing of yeast strain
M15532. YE (yeast extract) separation yield is the recovery
of solids from separation only (WF = 0) and of separation
plus one or two washes (WF = 1 or 2, respectively), relative
to the starting solids in the autolysate. YE MANU recovery is
the activity (in Phadebas MANU) from separation only (WF =
0) and of separation plus one or two washes (WF = 1 or 2),
relative to starting total Phadebas MANU in the autolysate.
Enzyme activity was determined in Phadebas enzyme assays with
comparison to a dose curve of the enzyme Novamyl ®
standards with known maltogenic amylase units (MANU).

| | YE | | |
|---|---|---|---|
| WASH | SEPARATION | YE MANU | |
| FACTOR | YIELD | RECOVERY | % DW |
| (WF) | (%) | (%) | in YE |
| 0 | 36 | 58 | 12.3 |
| 1 | 50 | 71 | 8.3 |
| 2 | 54 | 75 | 6.0 |

TABLE 9
Results of ultrafiltration of yeast extract of M15532 with a 10 kDa molecular weight cutoff.
YE is yeast extract, obtained by centrifuging fermenter autolysate in 1 liter bottles for
10 minutes at 11,000 RCF, to mimic separation at industrial scale. Retentate is the sample
retained by ultrafiltration and permeate is the sample not retained. Phadebas MANU/ml was
determined for each samples and MANU/g DW (dry weight) was calculated based on the dry weight
per sample. Enzyme activity was determined in Phadebas enzyme assays with comparison to a
dose curve of the enzyme Novamyl ® standards with known maltogenic amylase units (MANU).

| | Concentration | % DW | | | MANU | DW |
|---|---|---|---|---|---|---|
| Sample | factor | in sample | MANU/mL | MANU/g DW | balance (%) | balance (%) |
| YE | 1.0 | 11.6 | 1672 | 14422 | 100 | 100 |
| 10 kDa | 3.5 | 15.5 | 12583 | 83332 | 222 | 38 |
| RETENTATE | | | | | | |
| 10 kDa | | 10.9 | 22 | 203 | 1 | 67 |
| PERMEATE | | | | | | |

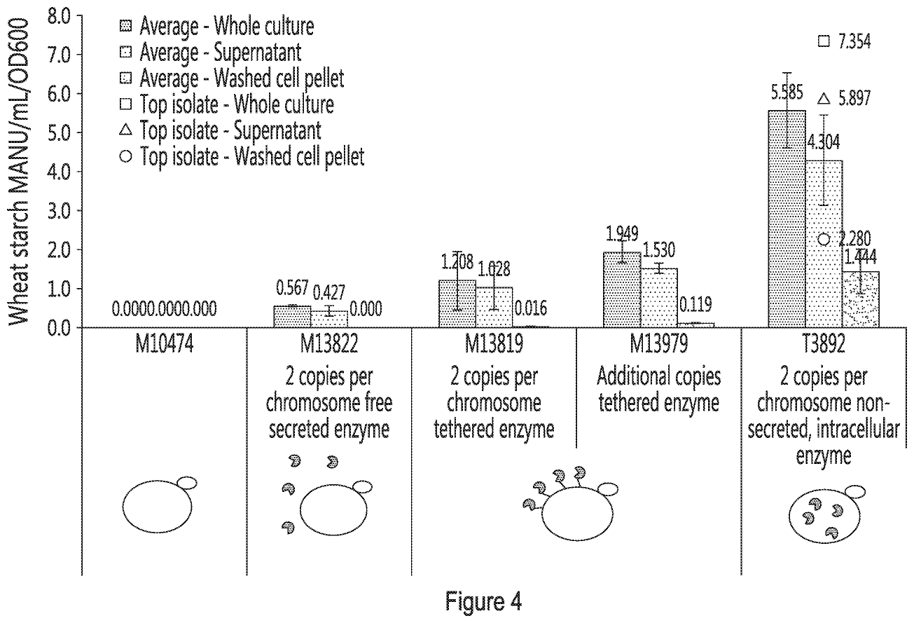

Various preparations of yeast strains expressing the maltogenic alpha amylase from G. stearothermophilus were made and their MANU activity was determined. Some strains expressed the MAA in a secreted form (M13822), other strains expressed the MAA in a tethered form (M13819 and M13979) while another strain expressed the MAA intracellularly (T3892). As seen in FIG. 4, the highest activities were observed in the strain expressing the MAA intracellularly (T3892).

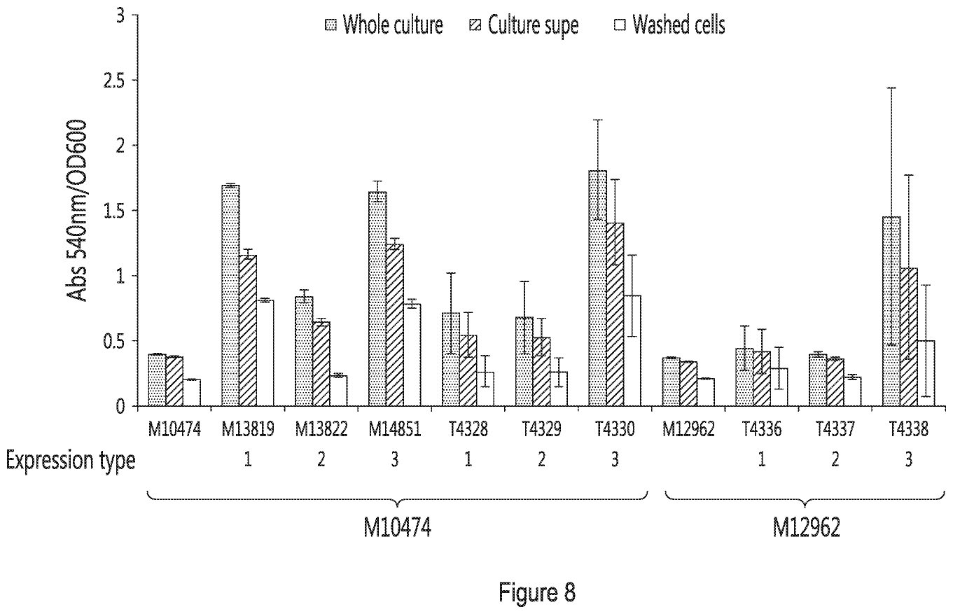

The wheat starch activity normalized to cell density was determined in the whole culture, the culture supernatant and the washed cells of various yeast strains expressing the maltogenic alpha amylase from G. stearothermophilus expressed in a secreted form, in a tethered form or expressed intracellularly as explained in the legend of FIG. 8. The results are shown in FIG. 8 and indicated that the highest activities are observed when the MAA is expressed intracellularly.

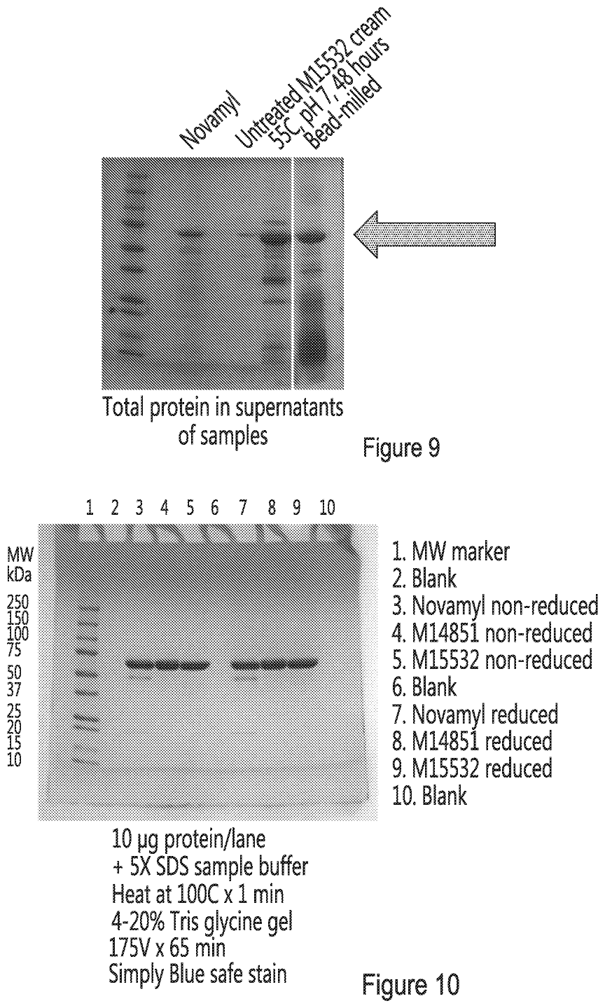

The protein content of the enzyme Novamyl® was compared on a SDS-page to the protein content of an untreated cream, a heat-treated cream and a bead-milled treated cream of the yeast strain M15532. The results are shown on FIG. 9. The arrow on FIG. 9 points to a major protein band seen in all treated preparations as well as in the enzyme Novamyl®.

On FIG. 10, the protein content of the enzyme Novamyl® was compared on a SDS-page to protein content derived from the yeast strains M14851 and M15532 under non-reducing conditions (lanes 3 to 5) and reducing conditions (lanes 7 to 9). A major protein band is seen in all the protein samples tested.

### Example III—Expression of Heterologous Alpha-Amylases, Glucoamylases, Phytases, Glucose Oxidases and Fungal Amylases

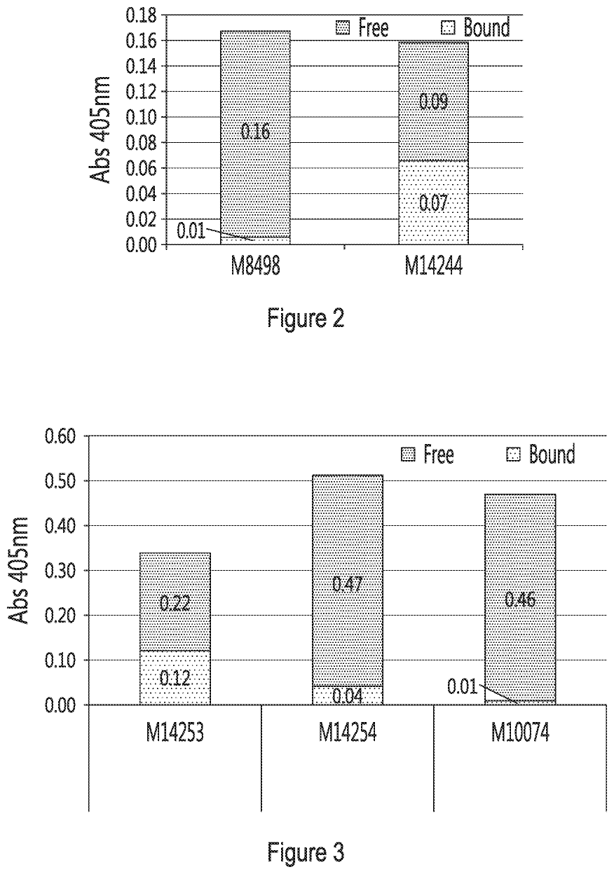

An heterologous glucoamylase (GA) was expressed in S. cerevisiae from the promoter of the tef2 gene. When GA was expressed as a tethered enzyme, activity associated with cellular pellet is increased (FIG. 2).

An heterologous alpha-amylase (AA) from the promoter of the tef2 gene. When the AA was expressed as a tethered enzyme, activity associated with pellet is increased, especially in the presence of a linker (FIG. 3).

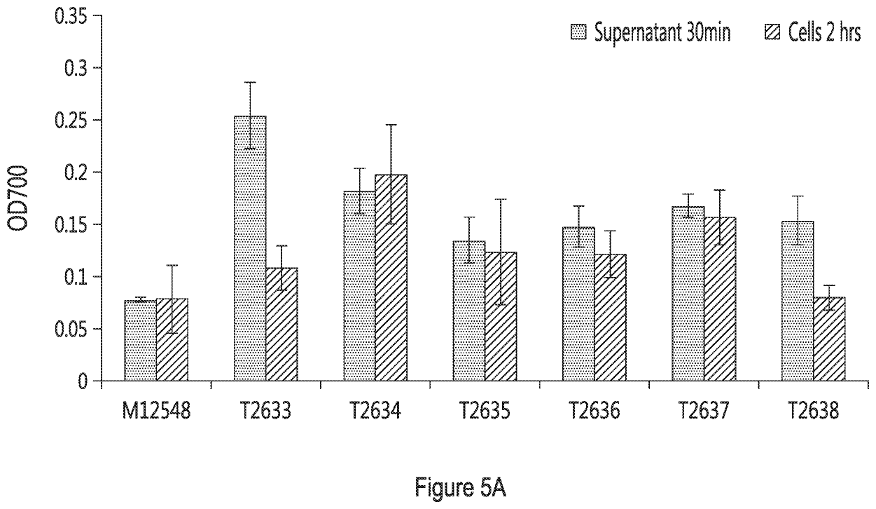

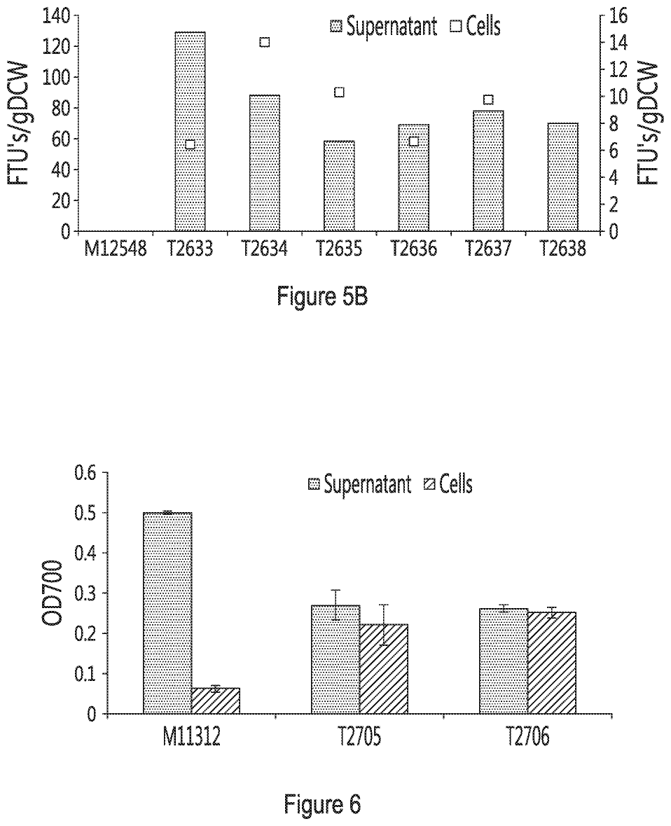

Various preparations of yeast strains expressing the phytase from C. braakii were made and their FTU activity was determined. Some strains expressed the phytase in a secreted form (T2633), other strains expressed the phytase in a tethered form (T2634, T2635, T2636, T2637 and T2638) using different tethers. The results are shown in FIGS. 5A and 5B for both the supernatant and the cells themselves.

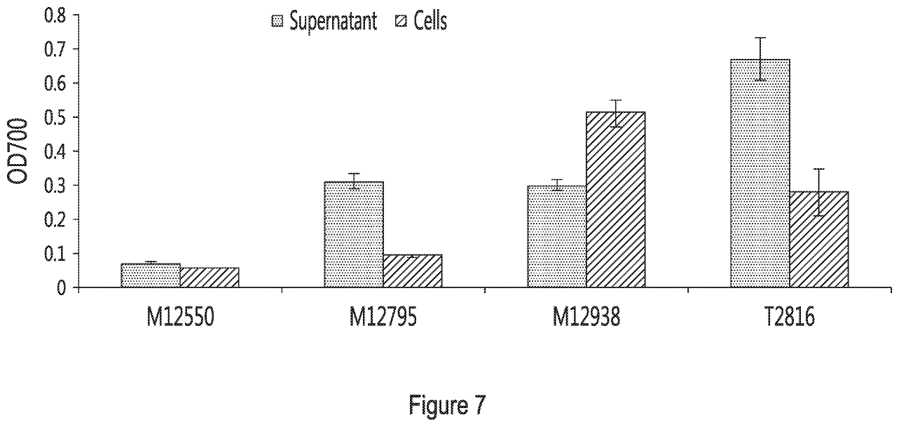

Various preparations of yeast strains expressing the phytase from E. coli were made and their FTU activity was determined. Some strains expressed the phytase in a secreted form (M11312), other strains expressed the phytase in a tethered form (T2705, T2706, M12795, M12938, T2816) using the different configurations of tethers. The results are shown in FIGS. 6 and 7 for both the supernatant and the cells themselves.

Heterologous chimeric thermo-tolerant P. furiosus alpha-amylase-SPI1 constructs and T. hydrothermalis alpha-amylase-CCW12 constructs were made using various truncations of the tethering moieties. The alpha-amylase activity associated with the washed cells of the strains expressing the chimeric polypeptides with the truncated GPI anchoring portions were compared to the non-truncated GPI anchoring portion is shown in FIGS. 12 and 13.

As seen from FIG. 12, the chimeric polypeptide with the full length tethering moiety (expressed from strain M15222) showed the same or higher alpha-amylase activity than the polypeptides with truncated tethering moieties (expressed from strains M15774 (21 aa-long truncation), M15771 (51 aa-long truncation), M1577 (81 aa-long truncation) or M15772 (130 aa-long truncation)).

As seen from FIG. 13, the chimeric polypeptides with the full length tethering moiety (expression from strain M15215) exhibited similar or higher alpha-amylase activity when compared to chimeric polypeptides having a truncated tethering moiety (expressed from strains M15773 (24 aa-long truncation), M15776 (49 aa-long truncation), M16251 (74 aa-long truncation) or M15775 (99 aa-long truncation)).

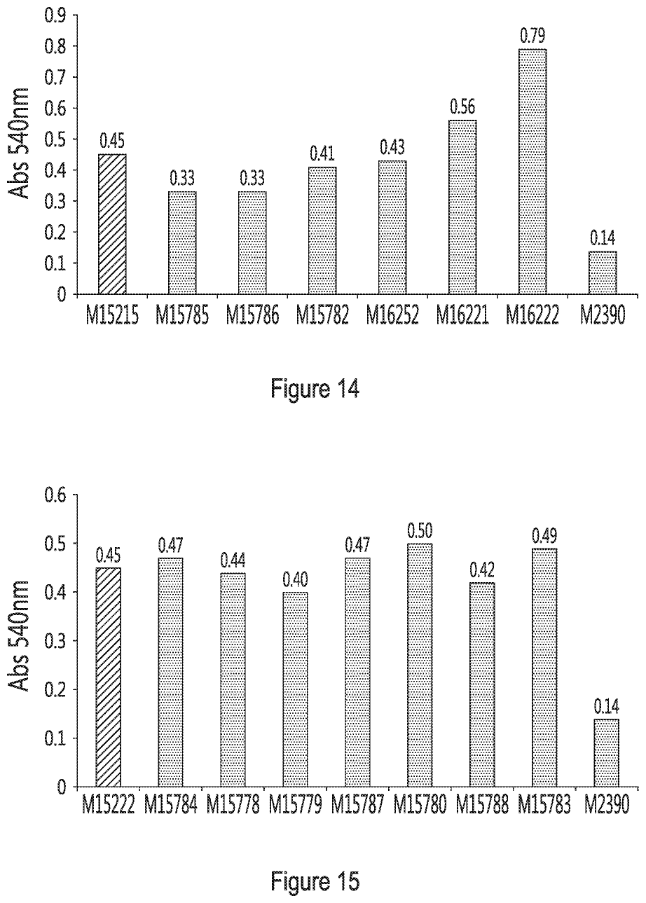

Heterologous chimeric thermo-tolerant P. furiosus alpha-amylase-SPI1 constructs and T. hydrothermalis alpha-amylase-CCW12 constructs were made using various linkers and the same tethering moiety. The alpha-amylase activity associated with the washed cells of the strains expressing the chimeric polypeptides with the different linkers is shown in FIGS. 14 and 15.

As seen from FIG. 14, the alpha-amylase activity of all the strains was higher than the control strain (M2390), irrespective of type of linker used. The alpha-amylase activity was the highest when linker 7 (SEQ ID NO: 99) was used (strain M16222).

As seen from FIG. 15, the alpha-amylase activity of all the strains was higher than the control strain (M2390), irrespective of type of linker used. The alpha-amylase activity was the highest when linker 5 (SEQ ID NO: 97) was used (strain M15780).

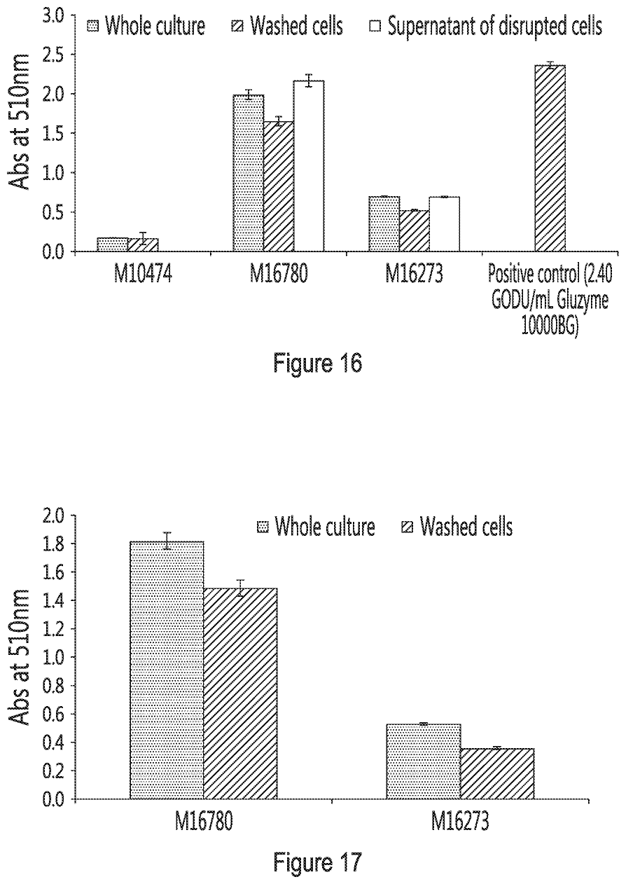

Heterologous chimeric glucose oxidase (GO) constructs were expressed intracellularly or in a secreted form. The GO activity obtained from various cellular fractions was compared to a control strain (M10474) or a positive control enzymatic preparation Gluzyme Mono® (FIG. 16). The GO activity associated with strains M16780 and M16273 was higher than the control GO activity associated with the parental strain M10474 (FIG. 17).

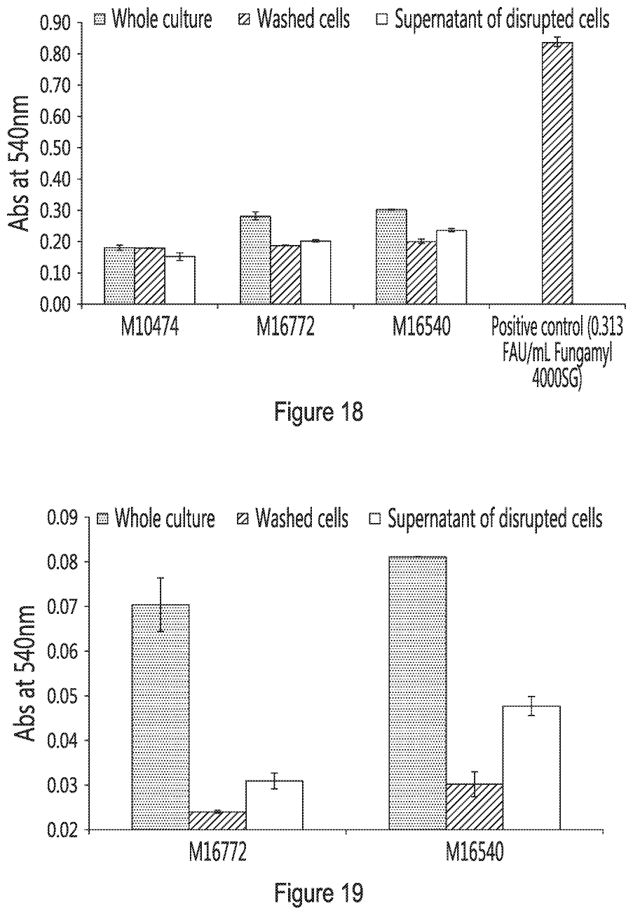

Heterologous chimeric fungal amylase (FA) constructs were expressed in a secreted form. The FA activity obtained from various cellular fractions was compared to control strain M10474 or a positive control enzymatic preparation Fungamyl® (FIG. 18). The FA activity associated with strains M16772 and M16540 was higher than the control activity associated with the parental strain M10474 (FIG. 19).

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Gascón S, Neumann N P, Lampen J O. Comparative study of the properties of the purified internal and external invertases from yeast. J Biol Chem. 1968 Apr. 10; 243(7):1573-7.
Pérez-Torrado R, Bruno-Bárcena J M, Matallana E. Monitoring stress-related genes during the process of biomass propagation of Saccharomyces cerevisiae strains used for wine making. Appl Environ Microbiol. 2005 November; 71(11):6831-7.
Praekelt U M, Meacock P A. MOL1, a Saccharomyces cerevisiae gene that is highly expressed in early stationary phase during growth on molasses. Yeast. 1992 September; 8(9):699-710.

1. A process for making a cell-associated heterologous protein from a recombinant yeast host cell, the process comprising:
a) propagating the recombinant yeast host cell in a medium placed in a vessel according to a baker's yeast production method so as to allow expression of the cell-associated heterologous protein, wherein the recombinant yeast host cell has an heterologous nucleic acid molecule encoding the cell-associated heterologous protein and the heterologous nucleic acid molecule is operatively associated with an heterologous promoter allowing expression of the heterologous nucleic acid molecule during propagation.

2. The process of claim 1, wherein the baker's yeast production method is a continuous method or a fed-batch method.

3. (canceled)

4. The process of claim 1, wherein a specific growth rate of the recombinant yeast host cell during the step of propagating is 0.25 h⁻¹ or less.

5. The process of claim 1 further comprising controlling an aeration rate of the vessel to at least about 0.5 or at least about 0.1 air volume/vessel volume/minute.

6. (canceled)

7. The process of claim 1, wherein the medium comprises a carbohydrate source, a nitrogen source and a phosphorous source.

8. The process of claim 7, wherein:
the carbohydrate source is derived from molasses, corn, glycerol and/or a lignocellulosic biomass;
the nitrogen source is ammonia; and/or
the phosphorous source is phosphoric acid;
the medium further comprises one or more micronutrients; and/or
the medium comprises molasses.

9.-11. (canceled)

12. The process of claim 5 further comprising controlling addition of a carbohydrate source to the medium so as to limit a growth rate of the recombinant yeast host cell.

13. The process of claim 12 comprising maintaining the concentration of the carbohydrate source at 0.1 weight percentage or less with respect to the total volume of the medium.

14. The process of claim 13, wherein the concentration of the carbohydrate source is maintained at 0.0001 weight percentage or less with respect to the total volume of the medium.

15. The process of claim 5 further comprising adding a nitrogen source and/or a phosphorous source to match a growth rate of the recombinant yeast host cell.

16. The process of claim 1 further comprising controlling pH of the medium to between about 4.0 and 5.0.

17. The process of claim 16 comprising controlling the pH of the medium at about 4.5.

18. The process of claim 1 further comprising controlling temperature of the medium to between about 20° C. to about 40° C. or between about 30° C. to about 35° C.

19. (canceled)

20. The process of claim 1, wherein, after the step of propagating, the recombinant yeast host cell is present at a concentration of at least 0.25 weight % of total volume of the medium.

21. The process of claim 20, wherein, after the propagation step, the concentration of the recombinant yeast host cell is of at least 1 weight % of the total volume of the medium.

22.-60. (canceled)

61. The process of claim 1, wherein the recombinant yeast host cell is from genus Saccharomyces sp or from species Saccharomyces cerevisiae.

62. (canceled)

63. The process of claim 1 for making a yeast composition, the process further comprising:
b) obtaining a propagated yeast host cell by said step of propagating and formulating the propagated yeast host cell into the yeast composition.

64. The process of claim 63, wherein the yeast composition is a cream yeast.

65.-67. (canceled)

68. The process of claim 1 for making a yeast product, the process further comprising:
b) obtaining a propagated yeast host cell by said step of propagating and lysing the propagated yeast host cell to obtain a lysed recombinant yeast host cell;
c) optionally drying the lysed recombinant yeast host cell to obtain a dried recombinant yeast host cell; and
d) formulating the lysed recombinant yeast host cell or the dried recombinant yeast host cell into the yeast product.

69. The process of claim 68, wherein step b) comprises submitting the propagated recombinant yeast host cell to autolysis to obtain the lysed recombinant yeast host cell.

70. The process of claim of claim 69, wherein step c) is conducted directly after step b) to provide an autolysate as the yeast product.

71. The process of claim 69, wherein the lysed recombinant yeast host cell comprises a soluble fraction and an insoluble fraction and the process further comprises, after step b), separating the soluble fraction from the insoluble fraction.

72. The process of claim 71 comprising:
filtering the lysed recombinant host cell to separate the soluble fraction from the insoluble fraction;
submitting the insoluble fraction to step d) to provide yeast cell walls as the yeast product;
submitting the soluble fraction to step d) to provide a yeast extract as the yeast product;
removing components having a molecular weight equal to or less than about 10 kDa from the soluble fraction to provide a retentate; and/or
submitting the retentate to step d) to provide a dry retentate as the yeast product.

73.-76. (canceled)

77. The process of claim 63, wherein the cell-associated heterologous protein is a heterologous enzyme.

78. The process of claim 68 further comprising:
e) substantially purifying the heterologous protein from the lysed recombinant yeast host cell to provide a purified heterologous protein as the yeast product.

79.-81. (canceled)

82. The process of claim 68, wherein the cell-associated heterologous protein is a heterologous enzyme.
